(19) United States
(12) Patent Application Publication
(10) Pub. No.: US 2025/0340934 A1
(43) Pub. Date: Nov. 6, 2025

(54) SELECTIVE BLOCKING TO DETECT AND AMPLIFY LOW ABUNDANT TEMPLATE

(71) Applicant: GT Molecular, Inc., Fort Collins, CO (US)

(72) Inventors: Sarah KANE, Fort Collins, CO (US); Mary STISCHER, Fort Collins, CO (US); Stephanie BARBARI, Fort Collins, CO (US)

(73) Assignee: GT Molecular, Inc., Fort Collins, CO (US)

(21) Appl. No.: 19/199,885

(22) Filed: May 6, 2025

Related U.S. Application Data
(60) Provisional application No. 63/643,014, filed on May 6, 2024.
(60) Provisional application No. 63/659,266, filed on Jun. 12, 2024.

(51) Int. Cl. C12Q 1/6848; C12Q 1/6818

(52) U.S. Cl. CPC C12Q 1/6848; C12Q 1/6818

(57) ABSTRACT

Provided herein are methods, kits, and related compositions useful for detecting from a biological sample a low variant allele frequency (VAF) by conventional PCR methods by use of two or more specially configured probes. A first universal-type labelled probe would, normally, when hybridized to a sequence, generate an optically-detectable signal by PCR. Provided herein, however, are specially configured oligonucleotides that, when bound, either prevents non-specific binding of labelled probe to the sequence and/or inhibits reference sequence synthesis by PCR. In this manner, even very low populations of sequences in the presence of another population of sequence that could differ by as little as one nucleotide, can be reliably detected by commercially-available PCR systems.

### CROSS-REFERENCE TO RELATED APPLICATIONS

This application claims the benefit of priority to U.S. Provisional Patent Application No. 63/643,014, filed May 6, 2024, and also to U.S. Provisional Patent Application No. 63/659,266, filed Jun. 12, 2024, each of which is hereby incorporated by reference in its entirety. INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One XML file named “1-24US_Seq_Listing_18July2025.xml,” created with WIPO Sequence on Jul. 18, 2025, of file size 358,892 bytes.

### BACKGROUND OF THE INVENTION

The following description provides a summary of information relevant to the present application and is not an admission that any of the information provided or publications referenced herein is prior art to the present application.

Provided herein are specially configured blocker oligonucleotides, referred to herein as mutant enhancing oligonucleotide wall (“MEOW”), with a 5′ exonuclease resister end and a 3′ extension blocker modification end that are useful for discriminating a low variant allele frequency (VAF) in a sample having a much higher frequency of wild type occurring alleles. This has important applications in both a screening assay application and a typing assay application.

Short nucleotide polymorphisms (SNPs), insertions, and deletions can drive cancer growth, allow pathogen immune invasion, and alter mRNA transcription, among others. These alterations in nucleic acid sequences may vary not only in the degree of nucleic acid changes, but also in prevalence. These challenges present difficulties in designing molecular assays in a few notable ways. First, cancer cells harbouring these mutations may represent a minor fraction of prevalence within a sample. As such, many reaction components, including primers, dNTPs, and enzymes, are effectively wasted on the relatively high wild-type or “background”, effectually drowning out the comparatively minor fraction of template that contains the mutation in the starting sample. Second, mutations may represent a very minor change, including down to a single nucleotide variation, rendering it challenging to design assays capable of discriminating a difference in a single nucleotide between a wild type reference (e.g., “normal”) sequence and a target (e.g., “mutant”) sequence. This difficulty is further exacerbated in certain regions of hypermutability, such as KRAS G12/G13 positions, that yield various mutations (G12A, G12C, G12D, G12R, G12S, G12V, G13C, and/or G13D).

US Pat. Pub. No. 2023/0250467 titled “Off-Target Blocking Sequences to Improve Target Discrimination by Polymerase Chain Reaction” (Kane et al.) discloses oligonucleotides (PBNJs) useful for improving target discrimination but is limited with respect to improved sensitivity related to the lack of a modification of the 5′ end of the probe. While PBNJs prevent nonspecific amplification detection via fluorescent probes, the unmodified 5′ end of the oligonucleotides are subject to hydrolysis by Taq polymerase. As such, while PBNJs prevent the detection of the reference sequence, the reference sequence continues to be amplified during PCR. There is a need in the art for oligonucleotides that avoid “wasting” reagents on a reference sequence because Taq cannot hydrolyse them. In essence, this prevents the exponential amplification of the reference sequence so that the lower abundant mutant sequence is provided the opportunity to be substantially amplified during PCR.

Drop-off assays have a generally similar approach wherein two oligonucleotides are used to target mutation region and reference region. Those assays, however, rely on two different fluorophores for signal detection with an attendant increase in assay complexity, reduction in targets per well, and associated increase in costs. Those assays do not, however, effectively drive the amplification toward the lower abundant target. See also bridged nucleic acid (BNA) polymerase chain reaction (PCR) clamping, including at (www.biosyn.com/bna-pcr-clamp-mutant-specific-probes.aspx); PCR U.S. Pat. No. 10,253,360 (BNA Clamp Method); A. S. Chubarov et al. “Allele-Specific PCR for KRAS Mutation Detection Using Phosphoryl Guanidine Modified Primers.” Diagnostics 2020 10(11): 872; Kabza, Adam M., et al. “Integration of chemically modified nucleotides with DNA strand displacement reactions for applications in living systems.” Wiley Interdisciplinary Reviews: Nanomedicine and Nanobiotechnology 14.2 (2022): e1743; How-Kit, Alexandre, et al. “Major improvement in the detection of microsatellite instability in colorectal cancer using HSP110 T17 E-ice-COLD-PCR.” Human Mutation 39.3 (2018): 441-453; WO2017201331 (“Oligonucleotide sequences for detection of low abundance target sequences and kits thereof”); 10,253,370; 10,329,605; 10,400,277; 11,208,689; WO2016172265; US20090176977.

There is a need in the art for compositions and related methods of using the compositions to solve problems related to: 1) specifically detecting minor nucleotide differences between two sequences; 2) detecting low abundant mutants within a sample mostly comprising wild type template; and 3) regions of hypermutability capable of generating numerous potential mutations, with the large number of potential mutations correspondingly increasing assay design complexity. The first two problems are generally characterized as being able to increase specificity and sensitivity. The latter problem is characterized as a complexity problem.

The invention provides such compositions and related methods. This and other advantages of the present invention will become apparent from the detailed description provided herein.

### BRIEF SUMMARY OF THE INVENTION

The present invention provides methods, kits, and compositions of matter useful for discriminating a low variant allele frequency (VAF) in a sample having a much higher frequency of wild type occurring alleles. Provided herein are specially configured blocker oligonucleotides, referred to herein as mutant enhancing oligonucleotide wall (“MEOW”), with a 5′ exonuclease resister end and a 3′ extension blocker modification end.

Provided herein is a method of screening for a target sequence from a reference sequence in a biological sample by polymerase chain reaction (PCR), the method comprising the steps of: providing a labelled probe comprising a fluorophore and a quencher, wherein the labelled probe has a shared sequence region configured to hybridize to a shared region of the target sequence and the reference sequence; providing a mutant enhancing oligonucleotide wall (MEOW) comprising: a 5′ exonuclease resister; a 3′ extension blocker configured to prevent elongation by a polymerase; a reference binding region positioned between the 5′ exonuclease resister and the 3′ extension blocker, wherein the reference binding region is configured to hybridize to a binding reference region of the reference sequence at a higher binding affinity than a corresponding binding target region of the target sequence; wherein the shared region of the target sequence and reference sequence is positioned downstream from: the binding reference region of the reference sequence; and the binding target region of the target sequence; performing a PCR on a PCR solution comprising: the biological sample containing the reference and/or target sequence; the labelled probe; the MEOW; PCR reagents; optically detecting an amplicon from the performing the PCR step; and identifying the biological sample as containing the target sequence for the optically detected amplicon; thereby screening for the target sequence from the reference sequence.

Provided herein is a method of typing a target sequence from a reference sequence in a biological sample by polymerase chain reaction (PCR), the method comprising the steps of: providing a labelled probe comprising a fluorophore and a quencher, wherein the labelled probe has a labelled probe sequence region configured to hybridize to a target sequence region of the target sequence; providing a MEOW comprising: a 5′ exonuclease resister; a 3′ extension blocker configured to prevent elongation by a polymerase; a reference binding region positioned between the 5′ exonuclease resister and the 3′ extension blocker, wherein the reference binding region is configured to hybridize to a binding reference region of the reference sequence; wherein: the labelled probe hybridizes to the target sequence region at a higher binding affinity than the MEOW non-specific binding to the target sequence region; and the MEOW hybridizes to the binding reference region at a higher binding affinity than the labelled probe non-specific binding to the binding reference region; performing a PCR on a PCR solution comprising: the biological sample containing the reference and/or target sequence; the labelled probe; the MEOW; forward and reverse primers; PCR reagents; optically detecting an amplicon from the performing the PCR step; and typing the biological sample as containing the target sequence for the optically detected amplicon; thereby typing the target sequence from the biological sample.

The MEOW-containing methods are demonstrated in both screening and typing assays. Importantly, we show efficacy across two unique gene targets with SNP sites as well as insertions. Furthermore, efficacy is explicitly demonstrated across two unique gene targets with SNP sites as well as insertions. This reflects that the instant methods and kits are applicable over a range of applications, including digital PCR and real time PCR.

Also provided herein are kits that are compatible with any of the methods or compositions disclosed herein. Disclosed is a kit for screening of a target sequence from a reference sequence in a biological sample by polymerase chain reaction (PCR), the kit comprising: at least one forward and reverse primer pair for amplifying both a reference strand having a reference sequence and a target strand having a target sequence; a labelled probe comprising a fluorophore and a quencher; a mutant enhancing oligonucleotide wall (MEOW); wherein the labeled probe hybridizes to the target sequence region at a higher binding affinity than the MEOW non-specifically binds to the target sequence region and the MEOW hybridizes to the binding reference region at a higher binding affinity than the labelled probe non-specific binding to the binding reference region; optionally: a positive control for the reference sequence; a positive control for the target sequence; and a mixed control comprising the reference sequence and the target sequence.

Provided herein is a kit for typing a target sequence from a reference sequence, the kit comprising: at least one forward and reverse primer pair for amplifying both a reference strand having a reference sequence and a target strand having a target sequence; a labelled probe comprising a fluorophore and a quencher; a mutant enhancing oligonucleotide wall (MEOW); wherein the labeled probe hybridizes to the target sequence region at a higher binding affinity than the MEOW non-specific binding to the target sequence region and the MEOW hybridizes to the binding reference region at a higher binding affinity than the labelled probe non-specific binding to the binding reference region; optionally: a positive control for the reference sequence; a positive control for the target sequence; and a mixed control comprising the reference sequence and the target sequence.

Any method or kit disclosed herein is compatible with a range of MEOW 5′ exonuclease resisters. In any method or kit disclosed herein, the MEOW 5′ exonuclease resister is optionally an Abasic site. Optionally, the MEOW 5′ exonuclease resister is consecutive locked nucleic acids (LNAs). Optionally, the MEOW 5′ exonuclease resister is consecutive phosphorothioate (PS) bonds. Optionally, the MEOW 5′ exonuclease resister is consecutive 2′-O-methoxyethyl (MOE) bases. Optionally, the MEOW 5′ exonuclease resister is consecutive 2′-O-Methyl (2′OMe). Optionally, the MEOW 5′ exonuclease resister is a hairpin. Optionally, the MEOW 5′ exonuclease resister is a biotin. Optionally, the MEOW 5′ exonuclease resister is a 2′ amino modification. Optionally, the MEOW 5′ exonuclease resister is a 2′ fluoro modification. Optionally, the MEOW 5′ exonuclease resister is a MOE and a PS. Optionally, the MEOW 5′ exonuclease resister is any combination of a LNA, PS, MOE, and a 2′OMe.

Optionally, the selection of the MEOW 5′ exonuclease resister is informed, at least in part, by cost, impact on hybridization, impact on MEOW melting temperature, whether the starting template is DNA or RNA, or any combination thereof. In aspects, the specific mechanism by which MEOW achieves its function (e.g., elimination of detection of non-specific amplification of a reference sequence), depends on the 5′ exonuclease resister. For example, disruption of the substrate (phosphodiester bond) required for nuclease activity (e.g., PS bonds), steric hindrance preventing nuclease access to the phosphate backbone (e.g., 2′-MOE or 2′Ome; hairpin), and/or increasing the binding affinity of the oligonucleotide to its target (e.g., LNA). Optionally, MEOW 5′ exonuclease resisters that change the internucleoside linkage confer greater nuclease resistance than the phosphodiester bond. Non-limiting examples of changes to internucleoside linkages include, carbophosphonate linkages (i.e., methylphosphonate, phenylphosphonate), methoxyphosphate, phosphonoformate, phosphonoaceate, thiophosphonoacetate, and/or mesyl phosphoramidate linkages. Optionally, MEOW 5′ exonuclease resisters described herein demonstrate increased duplex stability as compared to non-modified oligos, which is believed to contribute to nuclease resistance and specificity. Optionally, MEOW 5′ exonuclease resisters described herein alter the charge of the linkages and/or introduce chirality that is not sufficiently degraded by nuclease activity.

In any method or kit disclosed herein, the MEOW optionally inhibits nonspecific SNV probe hybridization of a reference sequence and blocks reference sequence amplification to detect a short nucleotide variant (SNV) of a target sequence. Optionally, the MEOW inhibits nonspecific SNV probe hybridization of a reference sequence and blocks reference sequence amplification to detect an insertion mutation of the target sequence. Optionally, the MEOW inhibits nonspecific SNV probe hybridization of a reference sequence and blocks reference sequence amplification to detect a deletion mutation of the target sequence. Optionally, the MEOW inhibits nonspecific SNV probe hybridization of a reference sequence and blocks reference sequence amplification to detect a fusion mutation of the target sequence.

In any method or kit disclosed herein, the MEOW optionally inhibits polymerase synthesis and the amplification of a wild-type sequence to provide a screening assay for various mutations located within close proximity from each other. In this context, close proximity optionally refers to mutations located within 1 to 50 nucleotides, 1 to 30 nucleotides, 1 to 25 nucleotides, or 1 to 20 nucleotides from each other. In this context, close proximity optionally refers to mutations located within a number of nucleotides corresponding to the number of nucleotides of the MEOW.

In any method or kit disclosed herein, the biological sample is optionally one or more viruses. Optionally, the reference sequence is from a wild-type virus or a parent virus and the target sequence comprises at least one mutation compared to the reference sequence. Optionally, the target sequence is from a wild-type virus or a parent virus and the reference sequence comprises at least one mutation compared to the target sequence. Optionally, in any method or kit disclosed herein, the biological sample is one or more mammalian cells. Optionally, in the case of mammalian cells, the reference sequence is reflective of a low-disease condition state and the target sequence has one or more nucleotide changes compared to the reference sequence reflective of an elevated disease condition. Optionally, the target sequence is reflective of a low-disease condition state and the reference sequence has one or more nucleotide changes compared to the reference sequence reflective of an elevated disease condition. Optionally, in any method or kit disclosed herein, the biological sample is circulating cell free or tumor DNA. Optionally, in the case of circulating cell free or tumor DNA, the reference sequence is somatic, wild-type sequence and the target sequence originated in a tumor or cancerous cell and has one or more nucleotide changes compared to the reference sequence reflective of an elevated disease condition risk or the presence of disease. Optionally, the target sequence is somatic, wild-type sequence and the reference sequence originated in tumor or cancerous cell and has one or more nucleotide changes compared to the target sequence reflective of an elevated disease condition risk or the presence of disease. Optionally, in any method or kit disclosed herein, the biological sample is circulating cell free fetal DNA. Optionally, in the case of circulating cell free fetal DNA, the reference sequence is reflective of the maternal DNA sequence and the target sequence has one or more nucleotide changes compared to the reference sequence reflective of a fetus DNA sequence. Optionally, the target sequence is reflective of the maternal DNA sequence and the reference sequence has one or more nucleotide changes compared to the target sequence reflective of a fetus DNA sequence. Optionally, in any method or kit disclosed herein, the biological sample is a bacteria. Optionally, in the case of bacteria, the reference sequence is from a wild-type bacterium or one species of bacteria and the target sequence comprises at least one variation compared to the reference sequence. Optionally, the target sequence is from a wild-type bacterium or one species of bacteria and the target sequence comprises at least one variation compared to the reference sequence. Optionally, in any method or kit disclosed herein, the biological sample is a fungus. Optionally, in the case of a fungus, the target sequence comprises at least one variation compared to the reference sequence. Optionally, the reference sequence comprise at least one variation compared to the target sequence. Optionally, in any method or kit disclosed herein, the biological sample is a plant. Optionally, in the case of a plant, the reference sequence is from a wild-type plant or one species of plant and the target sequence comprises at least one variation compared to the reference sequence. Optionally, the target sequence is from a wild-type plant or one species of plant and the reference sequence comprises at least one variation compared to the target sequence.

In any method or kit disclosed herein, the MEOW optionally eliminates greater than or equal to 50% detection of non-specific amplification of the reference sequence. Optionally, the MEOW eliminates greater than or equal to 55% detection of non-specific amplification of the reference sequence. Optionally, the MEOW eliminates greater than or equal to 60% detection of the non-specific amplification of the reference sequence. Optionally, the MEOW eliminates greater than or equal to 65% detection of the non-specific amplification of the reference sequence. Optionally, the MEOW eliminates greater than or equal to 70% detection of the non-specific amplification of the reference sequence. Optionally, the MEOW eliminates greater than or equal to 75% detection of the non-specific amplification of the reference sequence. Optionally, the MEOW eliminates greater than or equal to 80% detection of the non-specific amplification of the reference sequence. Optionally, the MEOW eliminates greater than or equal to 85% detection of the non-specific amplification of reference sequence. Optionally, the MEOW eliminates greater than or equal to 90% detection of the non-specific amplification of the reference sequence. Preferably, the MEOW eliminates greater than or equal to 95% detection of the non-specific amplification of the reference sequence. Preferably, the MEOW eliminates greater than or equal to 99% detection of the non-specific amplification of the reference sequence.

In any method or kit disclosed herein, the MEOW optionally provides an increase in fluorescent signal separation between an amplicon from a target sequence and an amplicon from a reference sequence. In aspects, the MEOW provides at least a 2-fold increase (e.g., at least a 2-fold increase, at least a 3-fold increase, at least a 4-fold increase, at least a 5-fold increase, or at least a 10-fold increase) in fluorescent signal separation between an amplicon from a target sequence and an amplicon from a reference sequence. In aspects, the MEOW provides between a 1-fold and 50-fold increase, between a 1-fold and 25-fold increase, between a 1-fold and 10-fold increase, between a 1-fold and 5-fold increase, between a 2-fold and 50-fold increase, between a 2-fold and 25-fold increase, between a 2-fold and 10-fold increase, or between a 2-fold and 5-fold increase.

Any method or kit disclosed herein is compatible with a range of target sequence mutations. For example, in any method or kit disclosed herein, the reference sequence and the target sequence optionally differ by a single nucleotide substitution. Optionally, the reference sequence and the target sequence differ by a nucleotide insertion or of one or more nucleotides. Optionally, the reference sequence and the target sequence differ by a nucleotide deletion of one or more nucleotides. Optionally, the reference sequence and the target sequence differ by a fusion construct.

In any method or kit disclosed herein, optionally, the reference sequence and the target sequence are DNA sequences. Optionally, the reference sequence and the target sequence are RNA sequences.

Any method or kit disclosed herein is compatible with a range of PCR assays. For example, any method or kit disclosed herein is compatible with ddPCR (droplet digital PCR), dPCR (digital PCR), qPCR (quantitative PCR), RT-ddPCR (reverse transcription-droplet digital PCR), RT-dPCR (reverse transcription-digital PCR), and RT-qPCR (reverse transcription-quantitative PCR).

In any method or kit disclosed herein, optionally, the PCR is real-time PCR.

Any method or kit disclosed herein is compatible with a range of labelled probes. For example, in any method or kit disclosed herein, optionally, the labelled probe is a dual-label probe comprising a fluorescent molecule and at least one quencher molecule. Any method or kit disclosed herein is compatible with a range of fluorescent molecules, such as FAM, HEX, VIC, SUN, TET, Yakima Yellow, Cy5, JUN, Cy5.5, TAMRA, ABY, ATTO550, Texas Red/ROX, ATTO590, and ATTO425. Any method or kit disclosed herein is compatible with a range of quencher molecules, such as Iowa Black FQ, Iowa Black RQ, and Black Hole Quencher1/2/3.

In any method or kit disclosed herein, the labelled probe optionally is an SNV-specific probe having a fluorophore covalently attached to the 5′ end. Optionally, the labelled probe comprises a 3′ end quencher. Optionally, the quencher is an internal quencher.

In any method or kit disclosed herein, the labelled probe optionally comprises a shared sequence region that is complementary to the reference and target shared regions.

Any of the methods or kits disclosed herein are compatible with the detection of a range of mutations. For example, any of the methods or kits disclosed herein are compatible with the detection of a SNV. Optionally, any of the methods or kits disclosed herein are compatible with detection of an insertion mutation. Optionally, any of the methods or kits disclosed herein are compatible with detection of a deletion mutation. Optionally, any of the methods or kits disclosed herein are compatible with detection of a fusion mutation. Optionally, any of the methods or kits disclosed herein are compatible with detection of mutation containing RNA. Optionally, any of the methods or kits disclosed herein are compatible with detection of a mutation containing DNA.

In any method or kit disclosed herein, the PCR is optionally dPCR. In any method or kit disclosed herein, the dPCR optionally comprises partition or droplet PCR. In any method or kit disclosed herein, the MEOW reduces or eliminates signal associated with a lower efficiency, non-specific off-target amplification, thereby increasing a signal to noise ratio for specific amplification of the target sequence.

In any method or kit disclosed herein, the method further comprises the steps of: tuning a probe output amplitude by providing the MEOW at a lower concentration than the concentration of the labelled probe. For example, the MEOW may optionally be provided at a concentration at is 0.1 times less than the concentration of the labelled probe. Optionally, the MEOW may be provided at a concentration that is 0.15 times less than the concentration of the labelled probe. Optionally, the MEOW may be provided at a concentration that is 0.2 times less than the concentration of the labelled probe. Optionally, the MEOW may be provided at a concentration that is 0.25 times less than the concentration of the labelled probe. Optionally, the MEOW may be provided at a concentration that is preferably 0.3 times less than the concentration of the labelled probe. Providing the MEOW at a concentration less than the concentration of the labelled probe may enable detecting a plurality of probe output amplitudes for multiplex detection of a plurality of target sequences in a single or a multichannel fluorescence detector.

In any method or kit disclosed herein, optionally the target sequence and the reference sequence differ by a single nucleotide mismatch that is a single nucleotide variant or is part of a short nucleotide variant.

In any method or kit disclosed herein, optionally, the target sequence and the reference sequence differ by an insertion.

In any method or kit disclosed herein, optionally, the target sequence and the reference sequence differ by a deletion.

In any method or kit disclosed herein, optionally, the target sequence and the reference sequence differ by a fusion event.

Any method or kit disclosed herein is compatible with a range of extension blockers. For example, the extension blocker is optionally 3′ carbon-based spacer. Optionally, the 3′ carbon-based spacer is C3. Optionally, the 3′ carbon-based spacer is C6. Optionally, the 3′ carbon-based spacer is C12. Optionally, the 3′ carbon-based spacer is dT/ddT. Optionally, the 3′ carbon-based spacer is a 3′ quencher.

In any method or kit disclosed herein, optionally, the MEOW contains a locked nucleic acid (LNA) at a SNV position.

In any method or kit disclosed herein, hybridization is provided by sequences that are preferably at least 90% complementary to each other over a sequence length of between 10 to 50 nucleotides. Optionally, hybridization is provided by sequences that are preferably at least 91% complementary to each other over a sequence length of between 10 to 50 nucleotides. Optionally, hybridization is provided by sequences that are preferably at least 92% complementary to each other over a sequence length of between 10 to 50 nucleotides. Optionally, hybridization is provided by sequences that are preferably at least 93% complementary to each other over a sequence length of between 10 to 50 nucleotides. Optionally, hybridization is provided by sequences that are preferably at least 94% complementary to each other over a sequence length of between 10 to 50 nucleotides. Optionally, hybridization is provided by sequences that are preferably at least 95% complementary over a sequence length of between 10 to 50 nucleotides. Optionally, hybridization is provided by sequences that are preferably at least 96% complementary over a sequence length of between 10 to 50 nucleotides. Optionally, hybridization is provided by sequences that are preferably at least 97% complementary over a sequence length of between 10 to 50 nucleotides. Optionally, hybridization is provided by sequences that are preferably at least 98% complementary over a sequence length of between 10 to 50 nucleotides. Optionally, hybridization is provided by sequences that are preferably at least 99%.

In any method or kit provided herein, the method further comprises providing a plurality of labelled probes comprising different fluorophores and quenchers specific to an application. Optionally, the method further comprises providing a plurality of MEOWs comprising different 5′ exonuclease resisters and 3′ extension blockers specific to an application. Optionally, the method further comprises providing a plurality of MEOWs and a plurality of labelled probes. Optionally, the plurality of MEOWs are provided such that the plurality of MEOWs hybridize to every possible SNV at the binding reference region, thus eliminating signal of the reference sequence.

Any method or kit provided herein is compatible with a range of biological samples. For example, any method or kit provided herein is compatible with a biological sample that allows to test for mutations associated with an elevated risk or presence of a disease condition associated with a predictive nucleotide sequence. For example, the predictive nucleotide sequence may indicate cancer, a neurodegenerative condition, or a reproductive condition.

Any method or kit provided herein is compatible with a range of biological samples. For example, a specific biological sample may allow to test for a variant of a pathogen. Pathogens may include, but are not limited to, a virus, a bacteria, or a fungus.

Any method or kit provided herein is compatible with a range of biological samples. For example, biological samples may include, but are not limited to bodily fluid, tissue, cell culture, plants, or tumors.

Any method or kit provided herein is compatible with a range of labelled probe polynucleotide sequences and MEOW polynucleotide sequences. The labelled probe polynucleotide sequence may differ from the MEOW polynucleotide sequence by one or more nucleotides.

In any method or kit provided herein, the MEOW is configured to favor amplification of an amplicon containing one or more mutations sought to be detected.

In any method or kit provided herein, a ratio of MEOW concentration to labelled probe concentration is equimolar or greater. Optionally, a ratio of MEOW concentration to labelled probe concentration is less than equimolar.

In any method or kit provided herein, optionally, a limit of detection for a mutation compared to wild-type or parent is 0.1% variant allele frequency. Optionally, a limit of detection for a mutation compared to wild-type or parent is 0.01% variant allele frequency. Optionally, a limit of detection for a mutation compared to wild-type or parent is 0.001% variant allele frequency.

In any method or kit provided herein, the method further comprises the step of providing an anti-blocker to promote amplification of a proto-oncogene by decreasing MEOW hybridization to the binding reference region. In certain aspects, the anti-blocker is a primer sequence capable of binding to a SNP. Optionally, the anti-blocker is between 5 and 100 nucleotides in length, for example, between 5 and 100 nucleotides, between 5 and 50 nucleotides, or between 5 and 30 nucleotides in length. Optionally, the anti-blocker is between 10 and 30 nucleotides in length. Optionally, the anti-blocker is SEQ ID NO. 152 (TGG TAG TTG GAG CTG GTG A). Optionally, the anti-blocker is SEQ ID NO. 153 (ACT TGT GGT AGT TGG AGC TGA). Optionally, the anti-blocker is SEQ ID NO. 154 (GAA TAT AAA CTT GTG GTA GTT GGA GCT T).

In any method or kit provided herein, the anti-blocker is a primer sequence that hybridizes to a SNP site.

In any method or kit provided herein, the MEOW substantially inhibits wild-type amplicon synthesis during any of the PCR methods disclosed elsewhere herein.

In any method or kit provided herein, a forward primer is provided at a concentration of between 50 nM and 1100 nM. Optionally, between 100 nM and 1050 nM. Optionally, between 150 nM and 1000 nM. Optionally, between 150 nM and 950 nM. Optionally, between 200 nM and 900 nM. Optionally, between 250 nM and 850 nM. Optionally, between 300 nM and 800 nM. Optionally, between 350 nM and 750 nM. Optionally, between 400 nM and 700 nM. Optionally, between 450 nM and 650 nM. Optionally, between 500 nM and 600 nM. In any method or kit provided herein, a reverse primer is provided at a concentration of between 50 nM and 1100 nM. Optionally, between 150 nM and 1000 nM. Optionally, between 150 nM and 950 nM. Optionally, between 200 nM and 900 nM. Optionally, between 250 nM and 850 nM. Optionally, between 300 nM and 800 nM. Optionally, between 350 nM and 750 nM. Optionally, between 400 nM and 700 nM. Optionally, between 450 nM and 650 nM. Optionally, between 500 nM and 600 nM. In any kit or method disclosed herein, the labelled probe is provided at a concentration of between 20 nM and 800 nM. Optionally, between 40 nM and 780 nM. Optionally, between 60 nM and 760 nM. Optionally, between 80 nM and 740 nM. Optionally, between 100 nM and 720 nM. Optionally, between 120 nM and 700 nM. Optionally, between 140 nM and 680 nM. Optionally, between 160 nM and 660 nM. Optionally, between 180 nM and 640 nM. Optionally, between 200 nM and 620 nM. Optionally, between 220 nM and 600 nM. Optionally, between 240 nM and 580 nM. Optionally, between 260 nM and 580 nM. Optionally, between 280 nM and 560 nM. Optionally, between 300 nM and 560 nM. Optionally, between 320 nM and 540 nM. Optionally, between 340 nM and 520 nM. Optionally, between 360 nM and 500 nM. Optionally, between 380 nM and 540 nM. Optionally, between 400 nM and 520 nM. Optionally, between 420 nM and 500 nM. Optionally, between 440 nM and 480 nM. In any method or kit disclosed herein, the MEOW may be provided at a concentration that is between 0.25× and 16× the concentration of the labelled probe. Optionally, the MEOW may be provided at a concentration that is between 0.5× and 15.5× the concentration of the labelled probe. Optionally, the MEOW may be provided at a concentration that is between 0.75× and 15.25× the concentration of the labelled probe. Optionally, the MEOW may be provided at a concentration that is between 1× and 15× the concentration of the labelled probe. Optionally, the MEOW may be provided at a concentration that is between 1.25× and 14.75× the concentration of the labelled probe. Optionally, the MEOW may be provided at a concentration that is between 1.5× and 14.5× the concentration of the labelled probe. Optionally, the MEOW may be provided at a concentration that is between 1.75× and 14.25× the concentration of the labelled probe. Optionally, the MEOW may be provided at a concentration that is between 2× and 14× the concentration of the labelled probe. Optionally, the MEOW may be provided at a concentration that is between 2.25× and 13.75× the concentration of the labelled probe. Optionally, the MEOW may be provided at a concentration that is between 2.5× and 13.5× the concentration of the labelled probe. Optionally, the MEOW may be provided at a concentration that is between 2.75× and 13.25× the concentration of the labelled probe. Optionally, the MEOW may be provided at a concentration that is between 3× and 13× the concentration of the labelled probe. Optionally, the MEOW may be provided at a concentration that is between 3.25× and 12.75× the concentration of the labelled probe. Optionally, the MEOW may be provided at a concentration that is between 3.5× and 12.5× the concentration of the labelled probe. Optionally, the MEOW may be provided at a concentration that is between 3.75× and 12.25× the concentration of the labelled probe. Optionally, the MEOW is provided at a concentration that is between 4× and 12× the concentration of the labelled probe. Optionally, the MEOW is provided at a concentration that is between 4.25× and 11.75× the concentration of the labelled probe. Optionally, the MEOW is provided at a concentration that is between 4.5× and 11.5× the concentration of the labelled probe. Optionally, the MEOW is provided at a concentration that is between 4.75× and 11.25× the concentration of the labelled probe. Optionally, the MEOW is provided at a concentration that is between 5× and 11× the concentration of the labelled probe. Optionally, the MEOW is provided at a concentration that is between 5.25× and 10.75× the concentration of the labelled probe. Optionally, the MEOW is provided at a concentration that is between 5.5× and 10.5× the concentration of the labelled probe. Optionally, the MEOW is provided at a concentration that is between 5.75× and 10.25× the concentration of the labelled probe. Optionally, the MEOW is provided at a concentration that is between 6× and 10× the concentration of the labelled probe. Optionally, the MEOW is provided at a concentration that is between 6.25× and 9.75× the concentration of the labelled probe. Optionally, the MEOW is provided at a concentration that is between 6.5× and 9.5× the concentration of the labelled probe. Optionally, the MEOW is provided at a concentration that is between 6.75× and 9.25× the concentration of the labelled probe. Optionally, the MEOW is provided at a concentration that is between 7× and 9× the concentration of the labelled probe. Optionally, the MEOW is provided at a concentration that is between 7.25× and 8.75× the concentration of the labelled probe. Optionally, the MEOW is provided at a concentration that is between 7.5× and 8.5× the concentration of the labelled probe. Optionally, the MEOW is provided at a concentration that is between 7.75× and 8.25× the concentration of the labelled probe.

Optionally, selection of the optimal concentration of MEOW is informed, at least in part, by the effectiveness of the MEOW (e.g., eliminating detectability of a reference sequence), the detectability of the target sequence, cost, or any combination thereof. For example, in aspects, a lower concentration of MEOW may be preferred to keep the cost of the assay lower and/or if the detectability of the target sequence is impacted by the MEOW at higher concentrations. In aspects, a higher concentration of MEOW may be preferred to enhance MEOW functionality (e.g., eliminating detectability of a reference sequence). Optionally, in aspects employing a screening approach as described herein, a higher concentration of MEOW is preferred as compared to a typing approach as described herein.

Any method or kit disclosed herein may further comprise the reagents needed to perform dPCR, ddPCR, qPCR, RT-dPCR, RT-ddPCR, RT-qPCR, or RT-PCR on a biological sample. Such reagents will be understood by a person having skill in the art.

Any method or kit described herein may comprise providing assay reagents for a first reaction not comprising a MEOW and a second reaction comprising at least one MEOW. In aspects, said providing step helps to assess the impacts of MEOW on target sequence amplification and/or detectability of a reference sequence.

Any method or kit provided herein is compatible with detection of SARS-CoV-2 mutation detection. For example, a MEOW may be provided to a wild-type reference sequence of a SARS-CoV-2 mutation to distinguish between at least one parental virus and at least one variant of the at least one parental virus. For example, the variant of the parental virus may include, but is not limited to, the common Spike gene corresponding to residues HV69-70, R408, K417, L452, T478, N679, L704, Q954, and L981.

In any method or kit provided herein, the reference sequence may be a parental SARS-CoV-2 and the target sequence may be a variant of SARS-CoV-2. Optionally, the variant of SARS-CoV-2 is the Alpha variant. Optionally, the variant of SARS-CoV-2 is the Beta variant. Optionally, the variant of SARS-CoV-2 is the Gamma variant. Optionally, the variant of SARS-CoV-2 is the Delta variant. Optionally, the variant of SARS-CoV-2 is the Delta plus variant. Optionally, the variant of SARS-CoV-2 is the Mu variant. Optionally, the variant of SARS-CoV-2 is the Lambda variant. Optionally, the variant of SARS-CoV-2 is the Omicron variant. Optionally, the variant of SARS-CoV-2 is an Omicron subvariant.

In any method or kit provided herein, the reference sequence may be a proto-oncogene and the target sequence may have a mutation that converts the proto-oncogene to an oncogene indicative of a higher risk of developing cancer or indicative of the presence of cancer.

In any method or kit provided herein, optionally, the proto-oncogene is KRAS.

In any method or kit provided herein, optionally, the target sequence is a KRAS mutation at the 12^{th} or 13^{th} codon. For example, the mutation is optionally, G12C, G12A, G12D, G12R, G12S, G12V, G13C, or G13D.

In any method or kit provided herein, optionally, the MEOW may be provided at a concentration so that one or more non-specific amplification population is optically indistinguishable from a negative population.

In any method or kit provided herein, optionally, the MEOW has a length of between 50% to 150% of the labelled probe with respect to the number of nucleotides. Optionally, the MEOW has a length of between 75% and 125% of the labelled probe.

In any method or kit provided herein, a real time PCR assay is optionally a cancer assay. Optionally, the real time PCR assay is a pathogen assay. Optionally, the real time PCR assay is the presence of a herbicide gene in a plant.

Also provided herein are compositions of matter useful for carrying out any of the methods described herein.

The MEOWs provided herein differ from previously described oligonucleotides (promiscuity-blocking nucleotide juror oligonucleotide or “PBNJ”) having only a 3′ modification, including a C3 spacer modification (U.S. Pat. Pub. No. 2023/0250467). While those PBNJs provide greater specificity for SNP and indel reactions, they do not prevent wild type amplicon synthesis. As such, samples with mixed proportions of wild type and mutant templates naturally contain a bias for amplifying the higher proportion wild type amplicon. To address this problem, provided herein are MEOWs having an additional 5′ modification that is better configured to specifically inhibit wild type template synthesis while affording skewing the preference towards a low-abundant mutant amplicon. Multiple types of 5′ modifications, alone and/or in combination, are capable of inhibiting wild type template synthesis. Examples include abasic site, locked nucleic acid (LNA), phosphorothioate (PS) bond, 2′-O-Methoxyethel (2′-MOE), 2′-O-Methyl (2′OMe), and hairpin modifications, with such modifications further differentiating MEOWs from previously described blocker technologies (U.S. Pat. Pub. No. 2019/10253360, 2019/10400277B2 and A.U. Pat. 2007209481B2) and providing important technical benefits as described herein.

The described MEOWs are experimentally validated with demonstrated utility against various mutations, including regions of KRAS hyper mutability as well as insertions in NPMI. Importantly, the technology provided herein results in between a 1.6 to 64-fold increase in sensitivity compared to conventional assays, such as QIAGEN KRAS Therascreen® in vitro diagnostic. In this manner, any of the methods described herein may have MEOWs and labelled probes configured to detect a VAF that is as low as 0.1%, 0.2%, 0.4%, 0.5%, 0.8%, 1%, 2% or 5%, including a VAF that is down to between about 0.1% and 5%, and any subranges therefor, such as between 0.1% and 1%.

Any of the methods described herein may be used with real time-PCR. In some aspects, the methods described herein may be used with conventional digital PCR platforms, including the QIAGEN QIAcuity® Digital PCR System, BIO-RAD QX200 Droplet Digital PCR System, or the Roche Digital Light Cycler System, and quantitative PCR systems, including BioRad CFX96 and Thermo Scientific QuantStudio5.

In certain aspects, the MEOW may differentially hybridize to certain mutations (e.g. KRAS G12D). To circumvent these issues, we introduce an anti-blocker novel technology that out-competes the MEOW and results in G12D amplification.

The compositions, methods and related kits are compatible with any of a range of applications, including oncology assay design, pathogen variant detection and discrimination, plant SNP identification, among others. For example, additional applications include genetic testing (for different diseases such as Alzheimer's disease, cancer, cystic fibrosis, sickle cell anemia, Duchenne muscular dystrophy, thalassemia, Huntington's disease, rare diseases, and other diseases), and a range of applications (cancer diagnosis, genetic disease diagnosis, cardiovascular disease diagnosis, and others). Another important application is for detection of antimicrobial resistance (AMR) genes, where small changes in the microbe genes can lead to antimicrobial resistance. Furthermore, the compositions, methods and kits are compatible and applicable for both real time and digital PCR technologies and is compatible with next generation sequencing (NGS) technologies as well.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWING(S)

FIGS. 1A-1B schematically illustrate a screening approach using the instant MEOWs. In FIG. 1A, the MEOW (labelled with “C3” portion) inhibits synthesis by PCR associated with binding of the labelled probe (labelled “FAM-Q”). In particular, both MEOW and labelled probe bind the non-mutated sequence, whereby the MEOW prevents synthesis and amplification, and thereby detection, by PCR from the labelled probe binding. FIG. 1B illustrates that for a mutated sequence, the MEOW does not bind while the labelled probe continues to bind.

FIGS. 2A-211 illustrate a MEOW that targets KRAS G12/G13 hypervariable region to suppress wild type amplification. In each of FIGS. 2A-211, “HEX green” refers to the signal generated from the universal probe, which was labeled with HEX (green). FIG. 2A: Wells without MEOW results in detection of both hypervariable, which was labeled with FAM (blue), and is indicated as “FAM blue”, and universal. FIG. 2B illustrates an oligonucleotide that only has a 3′ C3 spacer (e.g., without any 5′ modification) (referred herein as “PBNJ”) and targets the hypervariable region, showing incomplete wild type suppression as reflected by the FAM, blue signal curve, indicated as “FAM blue”. FIG. 2C-211: MEOWs targeting the hypervariable region synthesized with different 5′ modifications in addition to the 3′ C3 spacer of FIG. 2B show complete wild type suppression, over at least 40 PCR amplification cycles. The various 5′ modifications are noted in each of the figures, including Abasic (FIG. 2C), locked nucleic acids (LNAs) (FIG. 2D), 5′-(5) phosphorothioate (PS) bonds (FIG. 2E), 5′-(3) 2′-O-methoxyethyl (MOE) bases (FIG. 2F), or a combination of 5′-(3) consecutive MOE bases and PS bonds (FIG. 2G), or a combination of 5′-MOE bases and PS bonds (FIG. 211).

FIGS. 3A-3D illustrate MEOWs that suppress wild type but not mutant amplification testing KRAS G12 template. In each of FIGS. 3A-3D, the universal probe is indicated as “HEX green”, the absence of MEOW is indicated as (−) GT-Blocker, and the presence of MEOW is indicated as (+) GT-Blocker. In each of FIGS. 3A-3B the wild-type KRAS G12 template is indicated as “blue”. FIG. 3A depicts results from an assay with wild-type (non-mutated) KRAS template in the absence of MEOW. FIG. 3B depicts results from an assay wild-type (non-mutated) KRAS template in the presence of MEOW. In each of FIGS. 3C-3D, the mutated KRAS G12 template is indicated as “blue”. FIG. 3C depicts results from an assay with both wild-type and mutated KRAS template in the absence of MEOW. FIG. 3D depicts results from an assay with both wild-type and mutated KRAS template in the presence of MEOW.

FIGS. 4A-4I illustrate the addition of MEOW allows for discrimination between wild type (WT) and mutated KRAS sequences, wherein the mutation is at a notably low VAF. In each of FIGS. 4A-4I, signal associated with universal probe is indicated as “HEX green” and signal associated with mutated KRAS sequence is indicated as “blue”. Contrived mutant DNA samples are spiked into WT gDNA at indicated percentage fraction yield, (0% Mutant (FIG. 4A), 1.2% G12V Mutant (FIG. 4B), 1.5% G12C Mutant (FIG. 4C), 1.95% G12R Mutant (FIG. 4D), 0.8% G12A Mutant (FIG. 4E), 1% G13C Mutant (FIG. 4F), 4.8% G12D Mutant (FIG. 4G), 4.28% G12S Mutant (FIG. 411), 4.8% G13D Mutant (FIG. 4I)) a detectable fluorescent signal (e.g., FAM signal) that are distinguishable from the 0% Mutant (e.g., all WT gDNA sample of FIG. 4A).

FIGS. 5A-5D illustrate impact of sequence specific anti-blockers on detectability of mutant sequences. In each of FIGS. 5A-5D, signal associated with universal probe is indicated as “HEX green”. In each of FIGS. 5C-5D, signal associated with mutated KRAS sequence is indicated as “blue”. FIG. 5A depicts KRAS-G12D mutant assay without an anti-blocker. FIG. 5B depicts KRAS-G12D mutant assay with an anti-blocker. FIG. 5C depicts wild type KRAS-G12 assay without an anti-blocker. FIG. 5D depicts wild-type KRAS-G12 assay with an anti-blocker.

FIGS. 6A-6B illustrate a MEOW that facilitates selective amplification of G12D template in droplet digital PCR. In each of FIGS. 6A-6B, signal associated with universal probe (i.e., signals associated with both WT and mutated KRAS-G12 sequences) is indicated as “HEX green” and signal associated with WT KRAS-G12 is indicated as “gray”. FIG. 6A depicts assay with only wild-type KRAS G12. FIG. 6B depicts assay with mutant KRAS G12D. Universal/nonspecific KRAS assay (HEX green) detects both WT and mutated KRAS genotypes.

FIGS. 7A-7B illustrate MEOWs targeting NPM1 insertion sites suppress WT amplification. FIG. 7A depicts an illustration of the impact of the four base pair NPM1 base pair insertion on the wild-type sequence, compare the non-mutated sample (top) to the NPM1 insertion mutation (bottom). FIG. 7B depicts results from assays without a MEOW (top) and with a MEOW having a 5′ LNA blocker (bottom) in the presence of a NPM1 insertion.

FIGS. 8A-8B schematically illustrate a SNP Typing embodiment, for a labelled probe that is FAM-Q (FIG. 8A) and Fluor-Q (FIG. 8B) with probe blocker, with the “X” (13) indicating mutation location. The MEOW can out-compete the mutant-specific probe (e.g., the “labelled probe”) for hybridization to a non-mutated sequence (FIG. 8A, top and FIG. 8B, top) to limit the non-mutated sequence amplification and detection. In contrast, for a mutant sequence, the mutant-specific probe (e.g., the labelled probe) outcompetes the MEOW (FIG. 8A, bottom and FIG. 8B bottom panel), resulting in amplification and detection of the mutant sequence.

FIGS. 9A-9D illustrate MEOWs that abolish non-specific amplification in a KRAS G12/13 mutation discrimination assay. In each of FIGS. 9A-9D, signal associated with universal probe is indicated as “Cy5.5 brown”. In each of FIGS. 9A-9D, the top panels correspond to wells having only wild-type KRAS-G12, and the bottom panels correspond to wells having both the wild-type sequence and multiple KRAS G12 mutant sequences. FIG. 9A depicts results from tests without MEOW (i.e., “(−) GT-Blocker”). FIG. 9B depicts results from tests with a MEOW with an abasic site modification (SEQ ID NO: 42). FIG. 9C depicts results from tests with a MEOW with a bulk LNA modification (SEQ ID NO: 41). FIG. 9D depicts results from tests with a MEOW with a 3′MOE modification (SEQ ID NO: 44).

FIGS. 10A-10H illustrate the MEOWs (having both 5′ and 3′ modifications) (FIGS. 10E-10H) provided greater sensitivity than an oligonucleotide having only a 3′ modification (e.g., an oligonucleotide having a C3 spacer, referred to herein as “C3 PBNJ” or “PBNJ”) (FIGS. 10A-10D). In each of FIGS. 10A-10H, signal associated with universal probe is indicated as “Cy5.5 brown”. FIG. 10A depicts results for probe competition/typing formulations designed with C3 PBNJ for 0% variant allele frequency (VAF) of G13C (FIG. 10A, top) and G13D (FIG. 10A, bottom) KRAS mutants. FIG. 10B depicts results for probe competition/typing formulations designed with C3 PBNJ for 0.1% VAF of G13C (FIG. 10B, top) and G13D (FIG. 10B, bottom) KRAS mutants. FIG. 10C depicts results for probe competition/typing formulations designed with C3 PBNJ for 1% VAF of G13C (FIG. 10C, top) and G13D (FIG. 10C, bottom) KRAS mutants. FIG. 10D depicts results for probe competition/typing formulations designed with C3 PBNJ for 0.1% VAF of G13C (FIG. 10D, top) and G13D (FIG. 10D, bottom) KRAS mutants. FIG. 10E depicts results for probe competition/typing formulations designed with MEOW for 0% variant allele frequency (VAF) of G13C (FIG. 10E, top) and G13D (FIG. 10E, bottom) KRAS mutants. FIG. 10F depicts results for probe competition/typing formulations designed with MEOW for 0.1% VAF of G13C (FIG. 10F, top) and G13D (FIG. 10F, bottom) KRAS mutants. FIG. 10G depicts results for probe competition/typing formulations designed with MEOW for 1% VAF of G13C (FIG. 10G, top) and G13D (FIG. 10G, bottom) KRAS mutants. FIG. 10H depicts results for probe competition/typing formulations designed with MEOW for 0.1% VAF of G13C (FIG. 10H, top) and G13D (FIG. 10H, bottom) KRAS mutants.

FIG. 11 is a table summary demonstrating that the instant MEOW technology affords 1.6 to 64-fold increase in sensitivity compared to QIAGEN Therascreen® in vitro diagnostic reported values.

FIG. 12 illustrates addition of a MEOW prevents mutant signal in wells containing only non-mutated DNA in digital PCR.

FIG. 13 illustrates the MEOW out-performs traditional C3 PBNJs at preventing non-specific amplification in wells containing only non-mutated ESR1.

FIG. 14 illustrates addition of a MEOW (GT-Blocker) prevents undesired signal from upstream probes.

FIGS. 15A-15E illustrate hybrid MEOW (GT-Blockers) contain both 2′-O-Methyl RNA bases and traditional DNA bases. No MEOW was used in FIG. 15A. SEQ ID NO: 58 corresponds to FIG. 15B. SEQ ID NO: 59 corresponds to FIG. 15C. SEQ ID NO: 60 corresponds to FIG. 15D. SEQ ID NO: 61 corresponds to FIG. 15E.

FIGS. 16A-16B illustrate hybrid MEOW containing both 2′-O-Methyl RNA bases and traditional DNA bases were applicable for typing assay configurations. SEQ ID NO: 63 corresponds to FIG. 16B.

FIGS. 17A-17D illustrate a combination of a MEOW (GT-Blocker) and C3 PBNJ results in an additive effect of circumventing non-specific amplification. FIG. 17A shows RFU as a function of PCR cycle for no MEOW and no PBNJ. FIG. 17B shows RFU as a function of PCR cycle for C3 PBNJ. FIG. 17C shows RFU as a function of PCR cycle for GT-blocker (MEOW) only. FIG. 17D shows RFU as a function of PCR cycle for GT-blocker (MEOW) and C3 PBNJ.

FIGS. 18A-18D illustrate additional designs for MEOWs (GT-Blockers) include 2′ Amino, 2′ Fluro, or hairpin modifications. In each of FIGS. 18A-18D, signal associated with universal probe is indicated as “Universal”. A multiplexed assay targeting KRAS G13C and G13D mutations shows mutant signal when only non-mutant template is present. FIG. 18A shows that without the GT-Blocker there is mutant signal in wells containing only non-mutated KRAS DNA. FIGS. 18B-18D show that with the GT-Blocker only the “Universal” signal is present in non-mutated DNA wells. FIG. 18B corresponds to 2′ Amino MEOW (SEQ ID NO: 149). FIG. 18C corresponds to 2′ Fluro MEOW (SEQ ID NO: 150). FIG. 18D corresponds to hairpin modifications MEOW (SEQ ID NO: 151).

### DETAILED DESCRIPTION OF THE INVENTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

In general, the terms and phrases used herein have their art-recognized meaning, which can be found by reference to standard texts, journal references and contexts known to those skilled in the art. The following definitions are provided to clarify their specific use in the context of the invention.

As used herein, “biological sample” refers to any material, biological fluid, tissue, or cell obtained or otherwise derived from an individual. This includes blood (including whole blood, leukocytes, peripheral blood mononuclear cells, buffy coat, plasma, and serum), dried blood spots (e.g., obtained from infants), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. In certain aspects, the biological sample is used to test for mutations associated with an elevated risk of disease. In certain further aspects, when testing for risk of disease, the reference sequence is reflective of a low-disease condition state and the target sequence has one or more nucleotide changes in the reference sequence reflective of an elevated disease condition risk or presence of disease. In certain aspects, the biological sample is used to test for mutations associated with an elevated risk of cancer, dementia and/or cardiovascular conditions. In certain aspects, the biological sample is used to test for a variant of a pathogen, including a pathogen that is a virus. For plant applications, the biological sample is used to test for the presence or absence of a resistance gene, including for resistance to an insect or a herbicide, and/or, screening seeds to breed for a plant trait. In certain further aspects, when testing for a variant of a virus, the reference sequence is from a wild-type virus or a parent virus and the target sequence comprises at least one mutation in the reference sequence. In certain aspects, the biological sample is from wastewater, environmental sample, bodily fluid, tissue, cell culture, or tumor.

“Screening” refers to distinguishing between two different polynucleotide sequences in a sample by PCR by use of two different probes that bind to two distinct locations on a sequence strand. A first probe is characterized as a “universal” or a “labelled probe” and is configured to bind to the sequence strand irrespective of whether the strand is characterized as having a “reference sequence” or a “target sequence.” In contrast, the MEOW is configured to preferentially bind to one of the reference or target sequence. Binding of the MEOW effectively prevents hydrolysis of the labelled probe so that there is no or minimal optical signal (e.g. approaching background signal). Without binding of a MEOW, amplification occurs with a corresponding optically detectable signal (e.g., statistically significantly different from background signal).

“Typing”, while functionally similar to “screening” in that two different polynucleotide sequences in a sample can be differentiated by PCR, relies on a different mechanism. Although two different probes remain, they are configured to bind at the same location on a sequence strand. Due to different sequences, the probes have different binding affinities, depending on whether there is a target sequence region or a binding reference region. Effectively, the MEOW out-competes the labelled probe for one sequence condition (so that there is no signal because there is no PCR amplicon generation), but not for a second sequence condition where the labelled probe out-competes the MEOW (so that there is a signal from PCR amplicon generation). In this manner, a mutated low allele variant can be reliably detected by standard PCR techniques.

As used herein, “non-specifically binds” refers to binding or hybridization of a binding agent which is not correlated with the specificity of the binding agent. In certain aspects, the binding agent is an oligonucleotide which non-specifically hybridizes to an oligonucleotide sequence which is not completely complementary to the sequence of the oligonucleotide binding agent.

As used herein, “promiscuity” refers to non-specific hybridization of nucleic acids.

As used herein, “quantitative PCR”, “qPCR” refers to a PCR-based technique that couples amplification of a target DNA sequence with quantification of the concentration of DNA in the reaction. qPCR can be applied to a sample which originally comprised DNA or a sample comprising complementary DNA obtained from reverse transcription of RNA.

As used herein, “reference sequence” refers to a nucleotide sequence selected as the basis for comparison to a target sequence. In certain aspects, a target sequence is a sequence which differs from a reference sequence by one or more single nucleotide polymorphism, fusion, insertion or deletion. The term reference sequence, however, is intended to be used broadly herein. For example, although use of PBNJs has applications toward the parental or wild type sequence, PBNJs can also be used for any mutation that occurs within the probe binding region. As an illustrative example, a PBNJ designed as a G12C probe that can also include a wild type PBNJ, can also include other different reference sequences, such as a G12A, G12R, G13D, etc., PBNJ. Most broadly, a “reference sequence” simply refers to a sequence with one or more nucleotide difference(s) compared to a target sequence.

As used herein, “target” or “target sequence” are used interchangeably to refer to a nucleic acid that hybridizes to a primer and can be detected and/or quantified by dPCR, RT-PCR and qPCR analysis. Target or target sequence is used broadly to refer to any oligonucleotide sequence of interest, including a sequence associated with a pathogen (e.g., virus or bacteria) and a sequence associated with a patient genome (e.g., a mammal, such as a human). A labelled probe binds specifically to the target or target sequence and, therefore, has complementarity to the target or target sequence. In certain aspects, a target or target sequence is an oligonucleotide sequence which differs from a selected reference sequence by one or more single nucleotide polymorphism, fusion, insertion or deletion. In certain aspects, the target or target sequence is between 10 and 50 nucleotides in length. In certain further aspects, the target or target sequence is a region of between 10 and 50 nucleotides, within a polynucleotide of substantially longer length. Of course, the methods and compositions provided herein are compatible with very large target sequences (e.g., up to millions of bases long in some cases). We preferably design the oligonucleotides to generate <1000 base pair amplicon sizes, with between 10-50 nucleotides long primers, probes, PBNJs, and MEOWs.

A target or target sequence in the presence of corresponding primers and probes, polymerase, optionally reverse transcriptase, nucleotides, and at a suitable pH, temperature, metal and ion concentration, will specifically hybridize to a single-stranded target sequence and initiate synthesis of a second strand complementary to the target. This amplification may be repeated by cycling of temperature for repeated denaturing, hybridizing and extension, thereby amplifying any target sequences.

A primer does not need to reflect the exact sequence of the template but must be sufficiently complementary to hybridize with a template. A person having skill in the art will understand a primer to be “sufficiently complementary” to hybridize with a template when its nucleotide sequence contains enough complementary base pairs to form stable hydrogen bonds with the corresponding region of the template strand to initiate DNA synthesis by a polymerase under the given conditions (e.g., annealing temperature, salt concentration, etc.). For example, in some embodiments, 1, 2, or 3 nucleotide mismatches in a 20-25 nucleotide primer may still allow hybridization. A person having skill in the art will further understand that mismatches at or near the 5′ end are more tolerable, whereas a cluster of mismatches at or near the 3′ end may prevent extension by a polymerase.

A primer may further comprise a “tail” comprising additional nucleotides at the 5′ end of the primer that are non-complementary to the template. Typically, the lengths of primers range between 7-100 nucleotides in length, such as 10-30, 15-60, 20-40, and so on, more typically in the range of between 15-35 nucleotides in length, and any sub-ranges thereof. Shorter primer molecules generally require cooler temperatures to form sufficiently stable hybrid complexes with the template. The term “primer site” or “primer binding site” refers to the segment of the target DNA to which a primer hybridizes. Typically, a set of primers is used for amplification of a target polynucleotide, including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. Useful primers may be designed in accordance with any of the teachings provided in U.S. Pat. No. 10,465,238, which is specifically incorporated by reference herein, including for different 5′ tail lengths to facilitate amplicon differentiation from a first target and a different target based on amplicon length.

As used herein, “sequence complementarity” refers to the standard arrangement of bases in nucleotides in relation to their opposite pairing, such as thymine being paired with adenine and cytosine being paired with guanine. In certain aspects, sequence complementarity is complete or exact complementarity at all base positions within an oligonucleotide. RNA has uracil instead of thymine.

As used herein, “hybridization” is used interchangeably with “specifically binds” and refers to hybridization between complementary oligonucleotides or sequences of nucleotides. In certain aspects, a probe is designed to be specific to a target region wherein the probe has a sequence which is complementary to the nucleotide sequence of the target region. In an aspect, the probe may be completely (e.g., 100%) complementary to the target. Of course, there is some tolerance to variation, including down to 90% or greater complementarity, including depending on the hybridization conditions (e.g., temperature, pH, etc.).

An “at least one nucleotide difference” refers to a pair of corresponding polynucleotide sequences that have at least one difference between two otherwise identical sequence regions. More specifically, the region corresponds to a region recognized by the probe. The phrase at least one nucleotide difference is used broadly to encompass any type of difference, including by insertion, deletion, fusion, or a change in nucleotide. The difference may correspond to a single nucleotide difference or more than one nucleotide difference. For example, at least one, at least two, at least three, at least four, at least five, or at least ten nucleotide differences. For example, between 1 and 500 nucleotide differences, between 1 and 400 nucleotide differences, between 1 and 300 nucleotide differences, between 1 and 250 nucleotide differences, between 1 and 100 nucleotide differences, between 1 and 50 nucleotide differences, or between 1 and 25 nucleotide differences. The difference may be a contiguous difference in nucleotide differences. The difference may be at multiple distinct positions along the polynucleotide sequence. For convenience, the sequences may be referred to generally as a “target” or a “reference” polynucleotide sequence, from which that at least one nucleotide difference is desirably detected from a to-be-tested “test sample.”

“Detecting” is used broadly herein to refer to methods that can identify and/or quantify within a target polynucleotide sequence. The methods herein preferably detect whether a difference is found from a sample and also quantify the differences. For example, for an application where the target polynucleotide is from a virus, one (e.g., a “first”) target polynucleotide sequence may correspond to a wild-type (e.g., “parental”) sequence, and another (e.g., a “second”) target polynucleotide sequence may correspond to a variant, wherein there is at least one mutation in the target polynucleotide sequence. Similarly, for a disease such as cancer, a first target polynucleotide sequence may correspond to a “normal” sequence and the second target polynucleotide sequence having a mutation that is associated with cancer. In this manner, the methods and kits are compatible with any polynucleotide sequence of interest that, with a change in sequence, there is an attendant impact or change in a state from a first state to a second state, including associated with function, a mutation conferring resistivity, disease, pathogenicity, risk factor, efficacy, diagnostic outcome, and the like.

As used herein, the term “about” represents an insignificant modification or variation of the numerical value such that the basic function of the item to which the numerical value relates is unchanged. In certain aspects, about indicates 90% of the stated value.

As used herein, “probe” refers to an oligonucleotide designed to be at least partially complementary to a target polynucleotide sequence of interest such that when combined with a hybridization reaction it can bind to the target. A person having skill in the art will understand a probe to be “at least partially complementary” to bind with a target polynucleotide sequence of interest when its nucleotide sequence contains enough complementary base pairs to form stable hydrogen bonds with the corresponding region of the target polynucleotide sequence of interest to be detected under the given conditions (e.g., annealing temperature, salt concentration, etc.). Mismatch tolerance varies depending on reaction conditions. For example, under low-stringency conditions (e.g., lower temperatures and/or higher salt concentrations), some mismatches (e.g., one, two, or three mismatches) may be tolerated. However, under high-stringency conditions, a single mismatch may reduce or eliminate probe binding. A probe may have more than one possible hybridization target and depending on reaction conditions, e.g. temperature, may bind to only one target, two targets with different hybridization efficiency or no targets.

As used herein, “PCR” or “Polymerase chain reaction” refers to the well-known technique of enzymatic replication of nucleic acids which uses thermal cycling for example to denature, extend and anneal the nucleic acids.

As used herein, “Sample polynucleotide” refers to a biological sample having a target polynucleotide sequence that is detected, including without and/or with the difference in target polynucleotide sequence. For example, the sample may comprise a mixture of polynucleotides containing different sequences at the loci of interest.

As used herein, “Target polynucleotide of interest” refers to a portion of a longer polynucleotide, including a portion that may or may not contain a relevant at least one nucleotide difference. The polynucleotide may comprise RNA or DNA. Optionally, RT-PCR may be performed on the RNA to generate DNA that is then subject to PCR.

Probes may be of any functional length. Without limitation to any particular embodiment, probes may be of 10 to 100 nucleotides in length, 15 to 90 nucleotides in length, 25 to 75 nucleotides in length, 30 to 50 nucleotides in length, 37 to 43 nucleotides in length or any combination thereof.

Probes may be labelled by any means known in the art. The label on the probes may be fluorescent. The light emitted by the label on the probes may be detectable in the visible light spectrum, in the infra-red light spectrum, in the ultra-violet light spectrum, or any combination thereof.

“Fusion construct” refers to a hybrid gene formed by chromosomal inversion, deletion, or translocation events.

As used herein, “anti-blocker” or “anti-MEOW” refers to a mutation-specific oligonucleotide sequence capable of out-competing a MEOW. In certain aspects, the anti-blocker results in the promotion of mutant amplification without impairing blocking of WT amplification. In certain aspects, the anti-blocker outcompetes MEOW. In certain aspects, the anti-blocker decreases MEOW hybridization to a reference sequence binding region. In certain aspects, the anti-blocker has a higher sequence complementarity to a specified sequence as compared to the MEOW. For example, if a MEOW is discovered to dampen signal for G13D DNA in an assay, then adding an anti-blocker with a higher sequence complementarity to G13D DNA is expected to outcompete the MEOW and prevent said dampening of signal of the G13D DNA in the assay. In preferred aspects, the anti-blocker is a primer. Without ascribing to any particular theory, it is believed that an anti-blocker in the form of a primer promotes efficiency of amplification of a specific sequence because it promotes a shorter amplicon. In certain aspects, the anti-blocker is an unlabeled probe.

As used herein, “MEOW” and “GT-Blocker” are interchangeable and refer to a mutant enhancing oligonucleotide wall with a 5′ exonuclease resister end and a 3′ extension blocker modification end that are useful for discriminating a low variant allele frequency (VAF) in a sample having a higher frequency of wild type occurring alleles.

As used herein, “competitive binding” refers to a hybridization-based process in which two or more molecules capable of hybridizing with the same or overlapping binding site on a target molecule, and the binding of one molecule affects or inhibits the binding of the other molecule(s). The outcome of competitive binding may be influenced by factors such as binding affinity, relative concentrations of molecules, and hybridization conditions.

As used herein, “exonuclease resister” and “exonuclease inhibitor” are used interchangeably and refer to any compound, molecule, or agent that reduces, impairs, and/or prevents the activity of one or more exonucleases. In aspects, the exonuclease resister is or comprises an oligonucleotide modification. In aspects, the exonuclease resister is or comprises an oligonucleotide modification that renders the oligonucleotide resistant to exonuclease activity. In aspects, the exonuclease resister prevents nucleolytic degradation through chemical changes to the nuclease substrate. In aspects, the exonuclease resister is or comprises an oligonucleotide modification that prevents nucleolytic degradation by steric hindrance to the phosphate backbone of the oligonucleotide.

As used herein, “out-compete” refers to the ability of one molecule to bind to a target molecule more effectively than another competing molecule under the same conditions, such that the binding of the first molecule substantially reduces or eliminates binding of the competing molecule. For example, a mutant-specific oligonucleotide probe may out-compete a wild-type oligonucleotide probe for binding to a target sequence if it has greater sequence complementarity or higher binding affinity, thereby preferentially occupying/binding to the target sequence.

As used herein, “output tuning” or “tuning an output amplitude” refers to an adjustment of signal intensity, magnitude, or strength in a system or assay. In the context of nucleic acid detection, amplitude tuning may involve modifying one or more parameters, such as probe concentration, binding affinity, hybridization conditions, or signal amplification efficiency, to control the relative signal levels associated with different binding events. For example, adjusting the concentration of a MEOW may tune the output of the fluorescent signal of the reference sequence. In certain aspects, tuning a probe output amplitude may be achieved by providing a MEOW at a lower concentration, such as at 0.3×, 0.2×, or lower of the concentration of the labeled probe.

As used herein, “substantially reduces,” “substantially eliminates”, “substantially inhibits”, and like terms refer to a decrease in level, signal, or occurrence of a specified interaction that is sufficient to produce a measurable or functionally meaningful effect in the context of the invention. In certain aspects, substantially reduces refers to a reduction of at least 90%, at least 95%, or at least 99% relative to a control or reference condition. For example, in the context of a screening assay, binding of a first molecule to a target sequence may substantially reduce binding of a second molecule to the target sequence if it lowers the second molecule's binding by an amount that impacts detection or performance of the assay. As will be understood in the context of the description and examples herein, in aspects, the terms “reduces,” “eliminates,” and “inhibits”, may refer to “substantially reduces”, “substantially eliminates”, and “substantially inhibits”, respectively.

As used herein, “variant allele frequency” refers to the relative frequency of alleles (variants of a gene) at a particular locus within a population, expressed as a fraction or percentage. For example, VAF may be used to determine the significance or prevalence of genetic variations within a population or to identify mutations in diseases such as cancer, where the VAF can indicate the presence or extent of a mutation within, for example, tumor cells compared to normal cells. In embodiments, the MEOW may result in the detection of a VAF of less than or equal to 5%. Optionally, in an embodiment, the MEOW may result in the detection of a VAF of less than or equal to 4%. Optionally, in an embodiment, the MEOW may result in the detection of a VAF of less than or equal to 3%. Optionally, in an embodiment, the MEOW may result in the detection of a VAF of less than or equal to 2%. Optionally, in an embodiment, the MEOW may result in the detection of a VAF of less than or equal to 1%. Optionally, in an embodiment, the MEOW may result in the detection of a VAF of less than or equal to 0.9%. Optionally, in an embodiment, the MEOW may result in the detection of a VAF of less than or equal to 0.8%. Optionally, in an embodiment, the MEOW may result in the detection of a VAF of less than or equal to 0.7%. Optionally, in an embodiment, the MEOW may result in the detection of a VAF of less than or equal to 0.6%. Optionally, in an embodiment, the MEOW may result in the detection of a VAF of less than or equal to 0.5%. Optionally, in an embodiment, the MEOW may result in the detection of a VAF of less than or equal to 0.4%. Optionally, in an embodiment, the MEOW may result in the detection of a VAF of less than or equal to 0.3%. Optionally, in an embodiment, the MEOW may result in the detection of a VAF of less than or equal to 0.2%. Optionally, in an embodiment, the MEOW may result in the detection of a VAF of less than or equal to 0.1%. Optionally, in an embodiment, the MEOW may result in the detection of a VAF of less than or equal to 0.09%. Optionally, in an embodiment, the MEOW may result in the detection of a VAF of less than or equal to 0.08%. Optionally, in an embodiment, the MEOW may result in the detection of a VAF of less than or equal to 0.07%. Optionally, in an embodiment, the MEOW may result in the detection of a VAF of less than or equal to 0.06%. Optionally, in an embodiment, the MEOW may result in the detection of a VAF of less than or equal to 0.05%. Optionally, in an embodiment, the MEOW may result in the detection of a VAF of less than or equal to 0.04%. Optionally, in an embodiment, the MEOW may result in the detection of a VAF of less than or equal to 0.02%. Optionally, in an embodiment, the MEOW may result in the detection of a VAF of less than or equal to 0.01%.

The MEOWs provided herein address the aforementioned problems in notable ways. In the context of low variant allele frequency (VAF), MEOWs are useful for decreasing signal generated for abundant wild type polynucleotide by skewing an amplification reaction toward amplicons containing the mutation of interest. The MEOWs and associated methods and kits are useful in a range of applications, including a screening assay and also for typing assays. The compositions, methods and kits described herein are useful for a range of gene targets, including those genes tending to have relevant SNP sites, fusions, insertions and/or deletions. Further benefits include maximizing sensitivity of mutation-specific molecular PCR assays, and the ability to screen a sample for various mutations within a single reaction, thereby reducing consumable costs.

Examples. The invention can be further understood by the following non-limiting examples. The examples are provided to illustrate some of the concepts described within this disclosure. While each example is considered to provide specific individual embodiments of composition and methods of preparation and use, none of the examples should be considered to limit the more general embodiments described herein.

In the following examples, efforts have been made to ensure accuracy with respect to numbers used (e.g., amounts, temperature, etc.) but some experimental error and deviation should be accounted for.

### Example 1: Screening

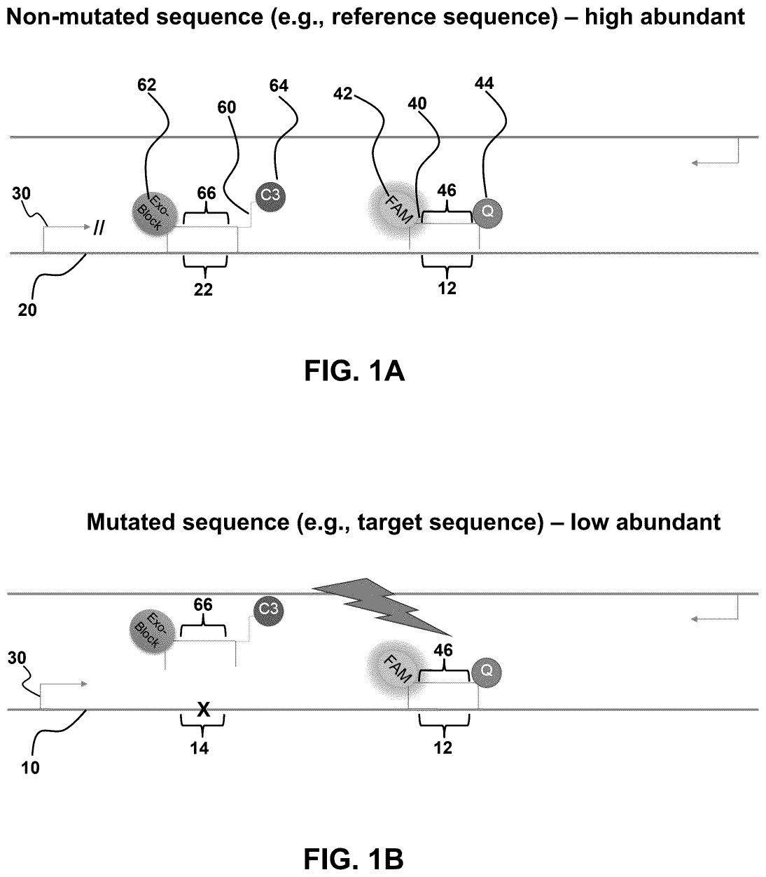

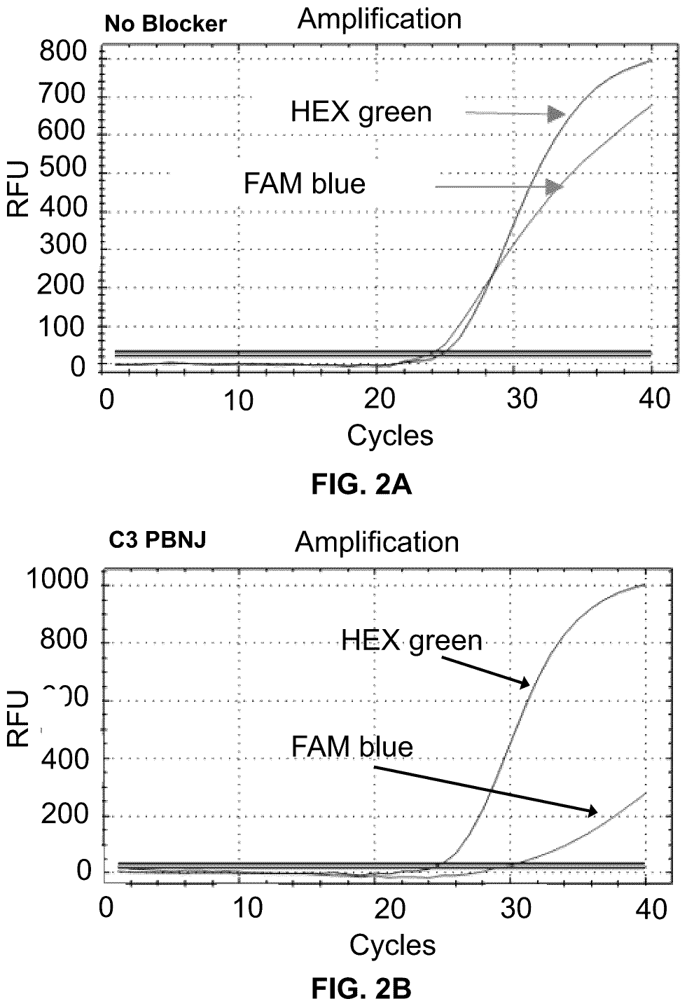

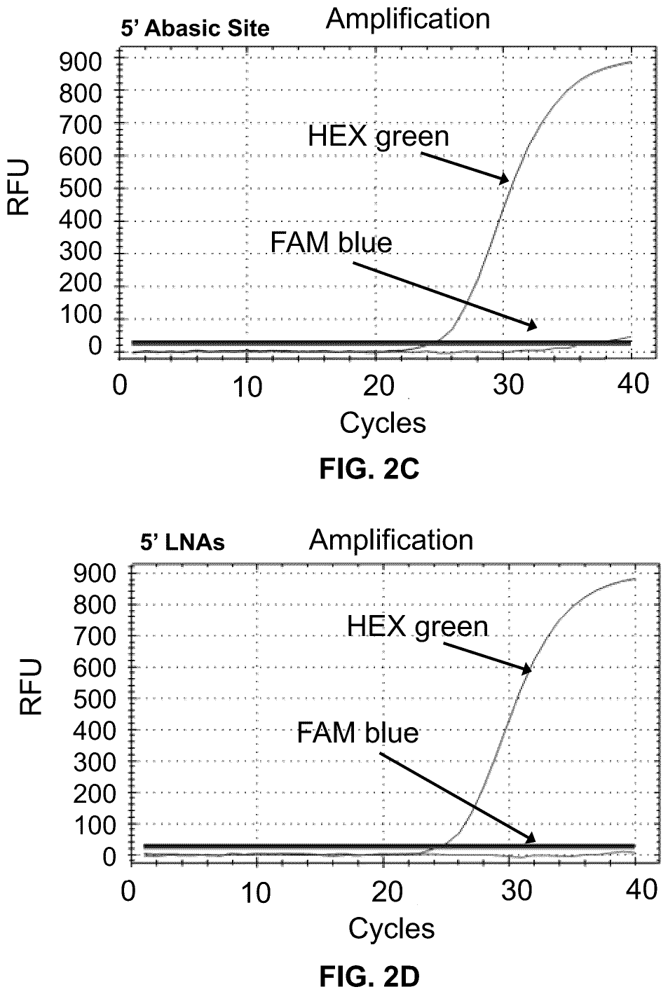

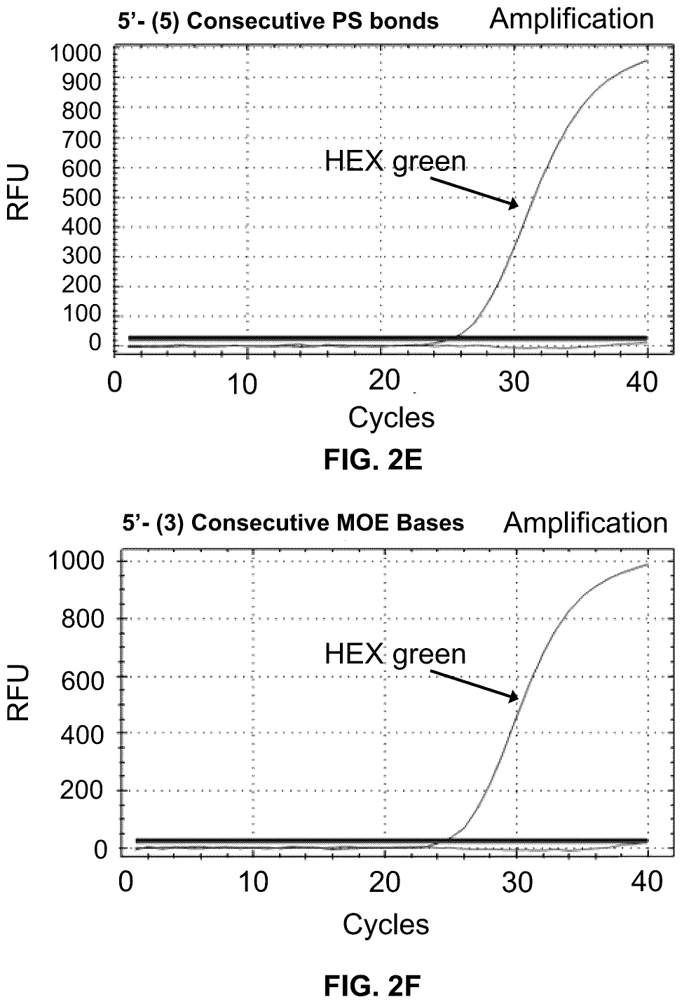

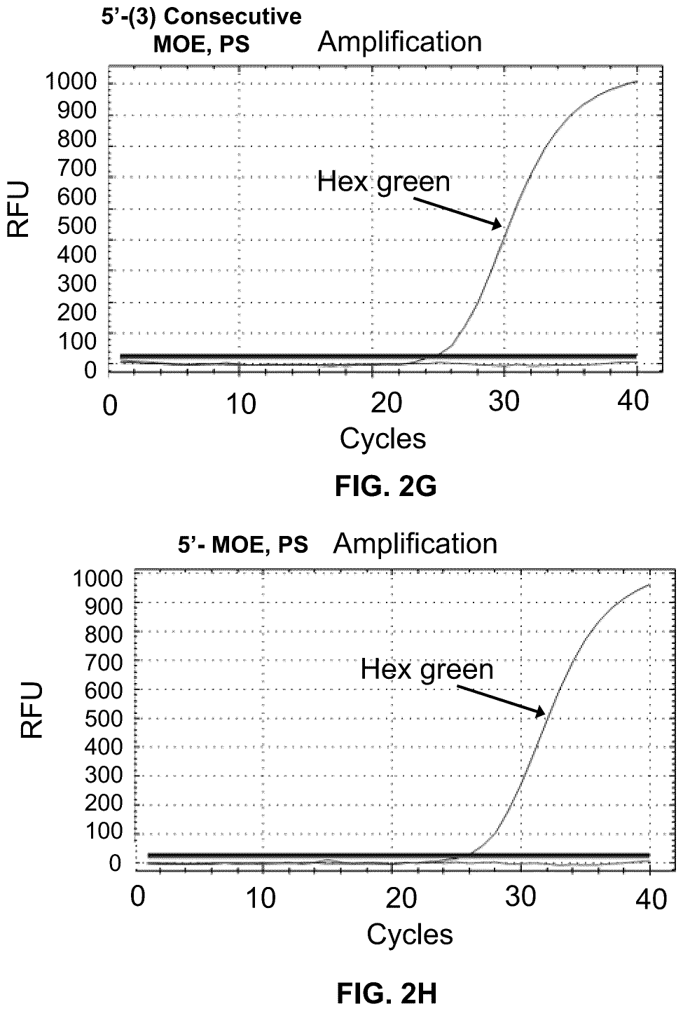

Screening approaches incorporates two probes: a “labelled probe” 40 and a “MEOW” 60 (see, e.g., FIGS. 1A-1B, illustrating binding of labelled probe irrespective of mutation status). The first labelled probe is designed to target any sequence regardless of mutation presence or absence and so is also referenced as a “universal probe” having an attendant universal signal (depicted as “Hex green”, FIGS. 2-5)). The first labelled probe 40 comprises a fluorophore 42 and a quencher 44. In addition, the labelled probe 40 has a shared sequence region 46 that is configured to hybridize to a shared region 12 of the target sequence 10 and the reference sequence 20. The second probe is referred herein as a MEOW 60 or a GT-Blocker, whose binding does depend on presence (represented as sequence 14 in FIG. 1B) or absence of a mutation (represented as sequence 22 in FIG. 1A). In an illustrated embodiment of FIG. 1A-1B, the MEOW 60 preferentially binds to wild type sequence 22 (FIG. 1A), and not to a sequence 14 having the mutation (FIG. 1B). As depicted in FIG. 1B, the MEOW does not hybridize in the presence of a mutation, and a detectable signal (e.g., fluorescence) is observed. The MEOW 60 comprises a 5′ exonuclease resister 62 and a 3′ extension blocker 64 and a reference binding region 66 positioned between the 5′ exonuclease resister 62 and the 3′ extension blocker 64. The reference binding region 66 is configured to hybridize to a binding reference region 22 of the reference sequence 20 at a higher binding affinity than a corresponding binding target region 14 of the target sequence 10. Accordingly, PCR synthesis and amplification for a detectable signal only occurs for a mutated sequence, with minimal or no detectable signal (e.g., no observed fluorescence) for wild type (non-mutated) sequence because the MEOW inhibits nonspecific SNV probe hybridization of the wild-type sequence and blocks wild-type sequence amplification. Accordingly, in this situation, synthesis via PCR can occur, with attendant fluorescence detection of the amplicon. In this manner, even for very high non-mutated sequence levels in a sample (compared to mutated sequence), fluorescence is observed so that the sample is correspondingly reliably screened to determine presence or absence of a mutation. This is effectively achieved for various 5′ modifications of probe blocker resulting in MEOW. For example, the system is validated for wild type G12/G13 with a downstream probe hybridization site. Only in the presence of a mutation that effectively results in little to no MEOW binding, will FAM (depicted as “FAM blue”, FIGS. 2-5) fluorescence be observed. Such FAM fluorescence is then an indication of presence of a mutated sequence.

FIGS. 2A-2H illustrate the significantly improved results for a MEOW with various 5′ modifications and a 3′ spacer configuration (see lack of detectable FAM, blue signal in FIGS. 2C-2H) compared to detected FAM, blue signal in FIG. 2B (3′ only modification (use of a C₃ PBNJ)) and FIG. 2A (no probe blocker)). Specifically, the MEOWs targeting the hypervariable region of were synthesized with different 5′ modifications in addition to the 3′ C3 spacer of FIG. 2B. The results in FIGS. 2C-2H show little, if any, detectable signal of the wild-type sequence, suggesting MEOW facilitates wild type suppression over at least 40 PCR amplification cycles. The various 5′ modifications are noted in each of the figures, including Abasic (FIG. 2C), locked nucleic acids (LNAs) (FIG. 2D), 5′-(5) phosphorothioate (PS) bonds (FIG. 2E), 5′-(3) 2′-O-methoxyethyl (MOE) bases (FIG. 2F), or a combination of 5′-(3) consecutive MOE bases and PS bonds (FIG. 2G), or a combination of 5′-MOE bases and PS bonds (FIG. 211).

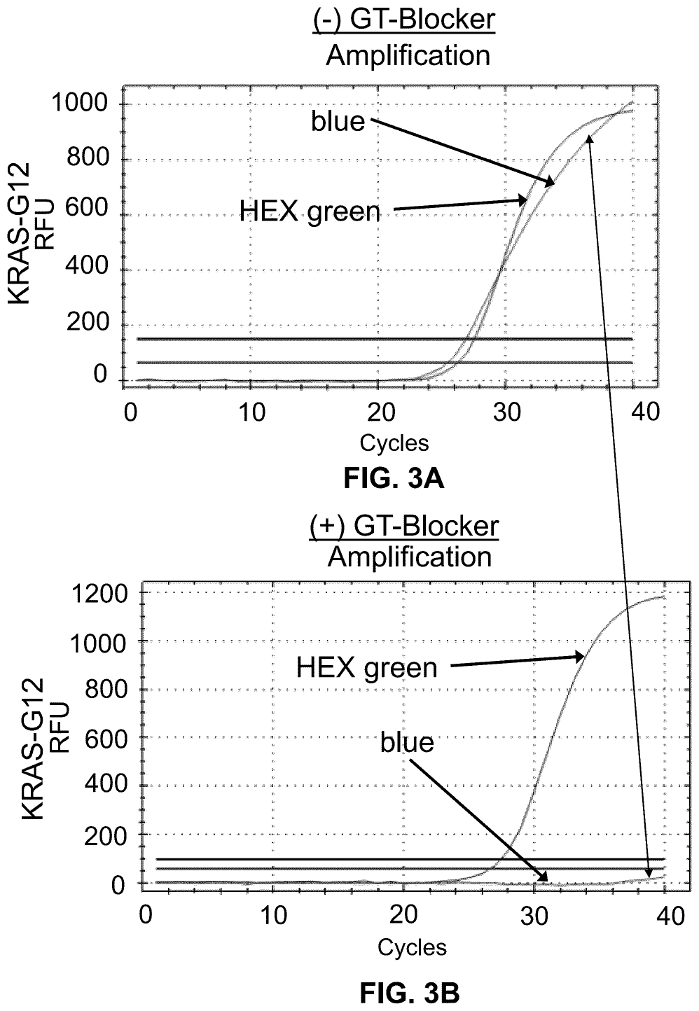

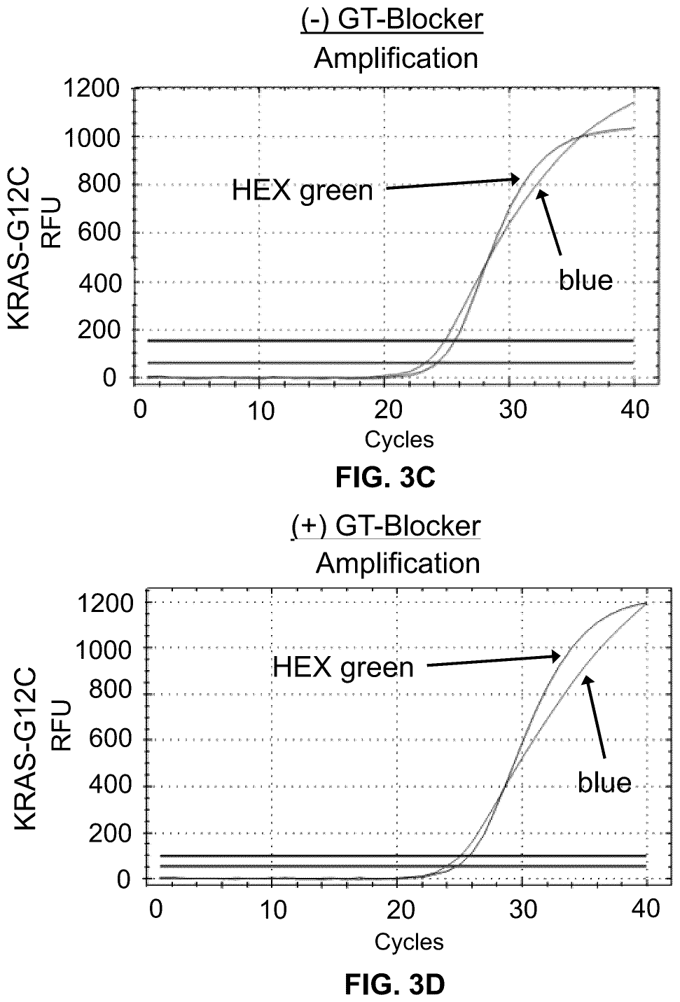

FIGS. 3A-3D confirms that the MEOW impacts only wild type amplification and not mutant amplification. Universal/nonspecific KRAS assay (HEX, Green) detects both wild type and mutated KRAS genotypes regardless of MEOW presence. FIG. 3A: In the absence of MEOW (as indicated by (−) GT-Blocker), wild type KRAS G12 template is detected with hypervariable assay, as indicated by the blue-labelled curve. FIG. 3B: In the presence of a MEOW (as indicated by (+) GT-Blocker), KRAS-G12 template is not detected, as indicated by lack of significantly detectable blue curve (as indicated by the double-sided arrow) showing the blue curves of FIG. 3A and FIG. 3B). FIGS. 3C-3D: Mutated KRAS G12C template is detected at similar CT values, regardless of absence (FIG. 3C) or presence (FIG. 3D) of MEOW, indicating that MEOW does not impact mutant amplification.

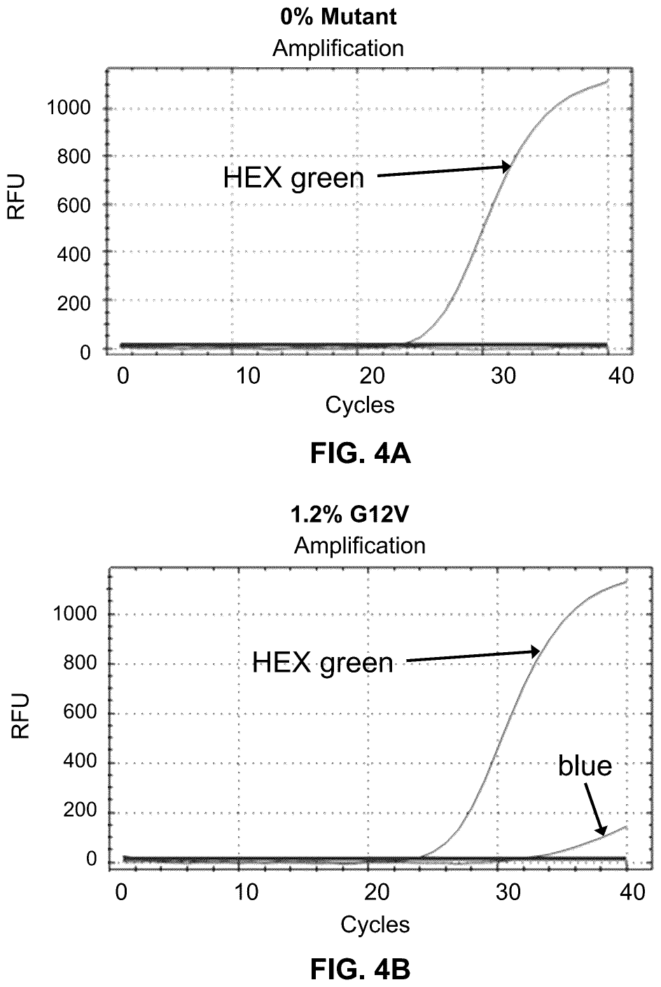

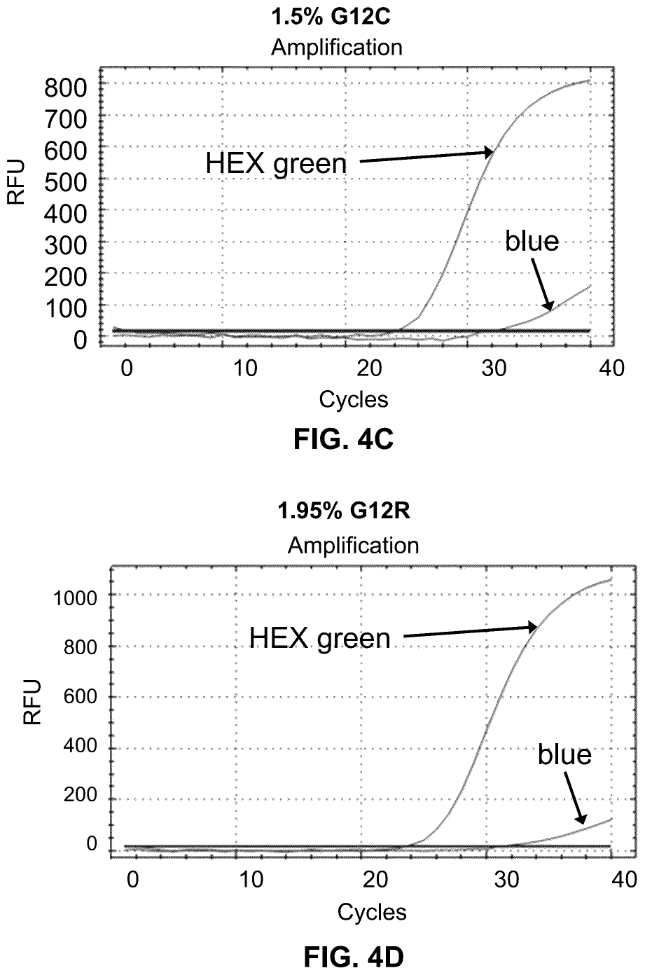

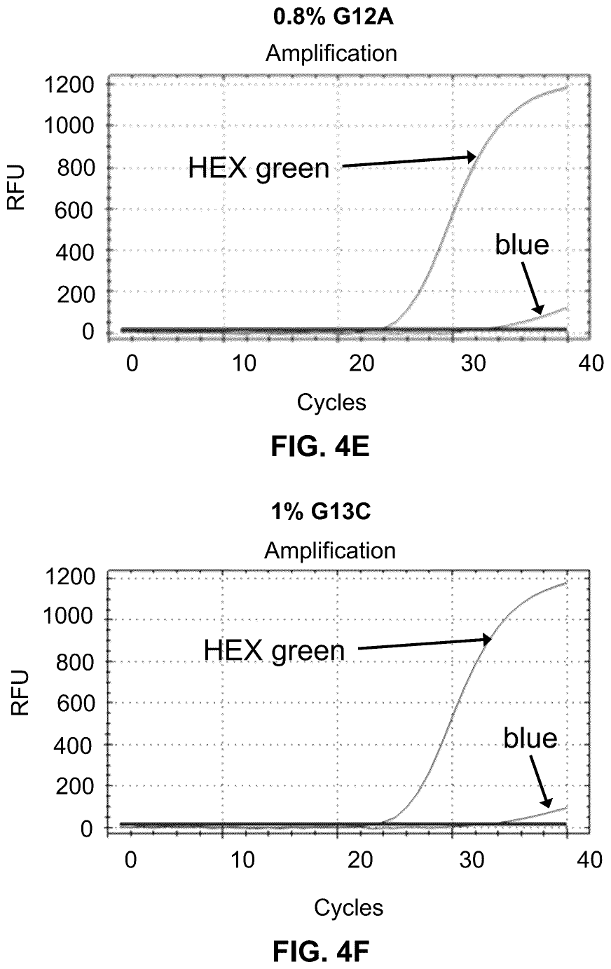

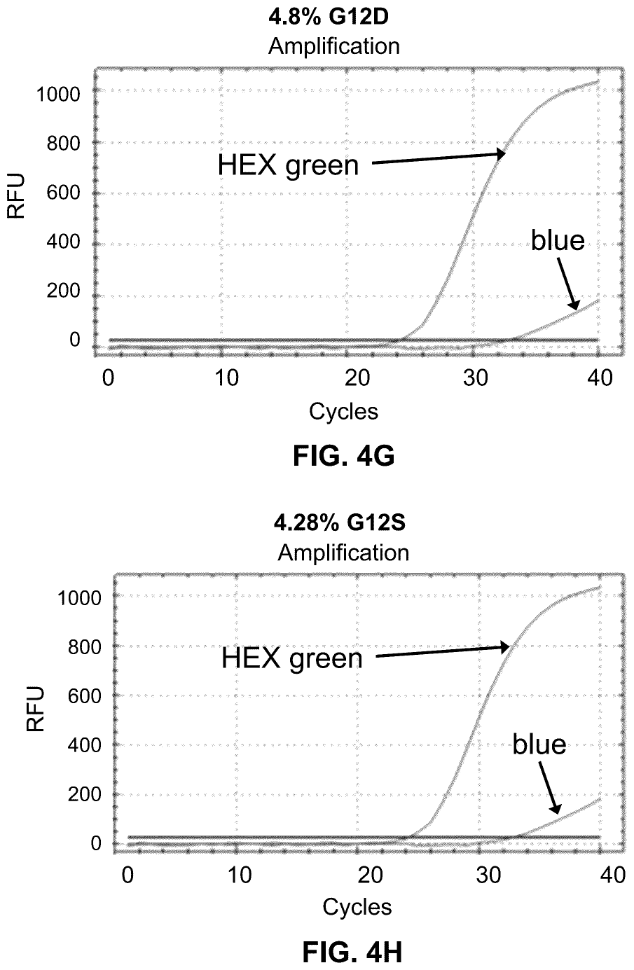

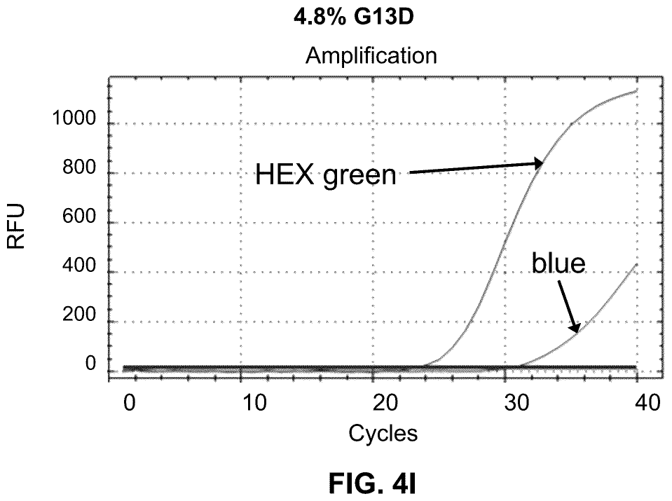

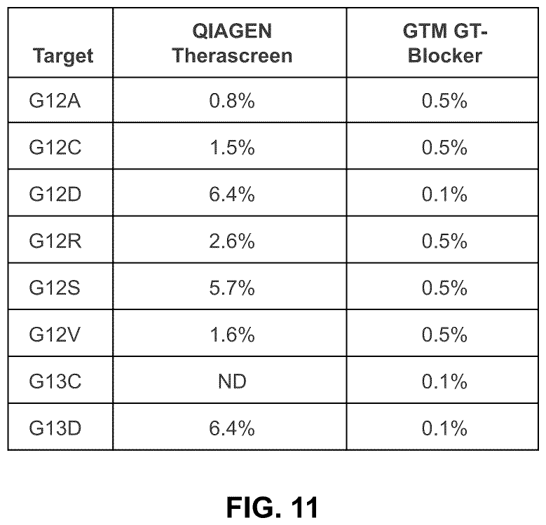

The current in vitro diagnostic manufactured by QIAGEN is able to detect ≤6.4% VAF (academic.oup.com/labmed/article/47/1/30/2505053). We then tested the ability to detect low VAF mutants. With MEOW technology in a screening assay approach, we were able to detect VAF as low as 0.8% (FIG. 4A-4I), and all mutants were detected at <5% VAF. See, e.g., FIG. 11 (detection as low as 0.1%). For example, mutants detected at lower than 3% VAF include G12V, G12C, G12R, G12A and G13C (compare FIG. 4A against FIGS. 4B-4F). Mutants detected at less than 5% VAF include G12S, G12D and G13D (FIGS. 4G-4I).

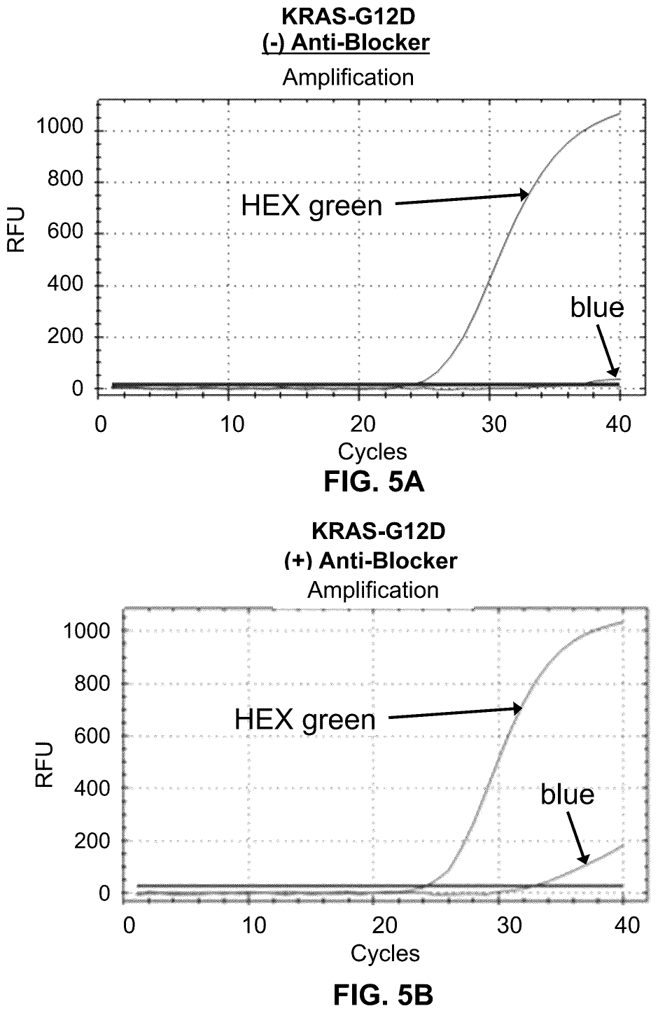

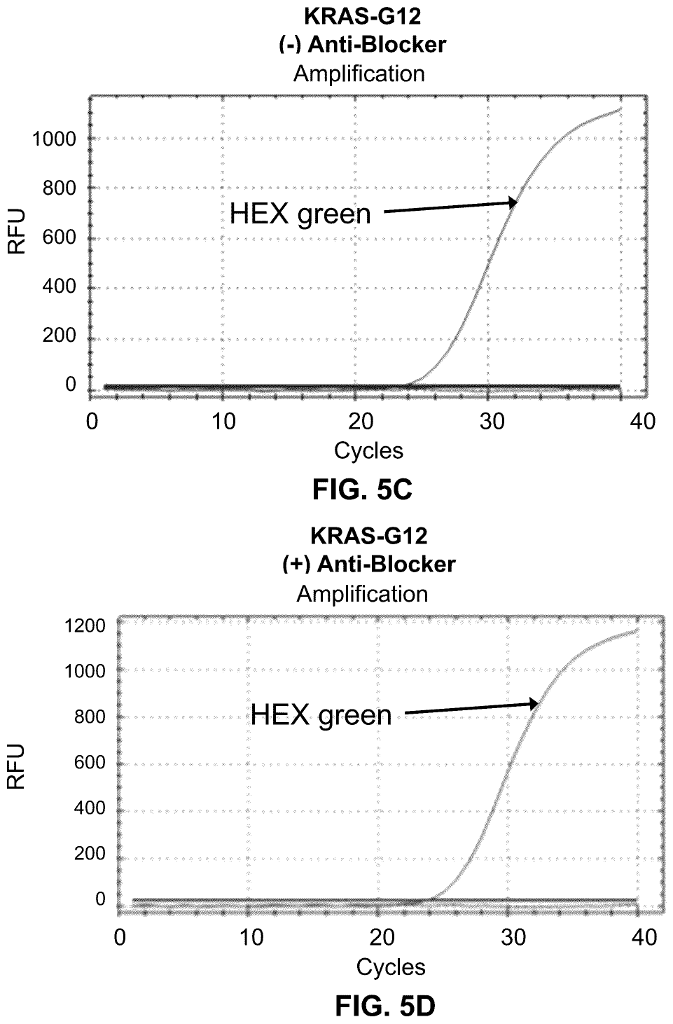

However, we noted that MEOWs tended to inhibit certain mutants, particularly KRAS G12D. We, therefore, utilize a technology termed Anti-MEOWs, to specifically promote the amplification of KRAS G12D (FIGS. 5A-5B), among others. As shown in FIGS. 5A and 5B, the addition of a mutation-specific anti-blocker for KRAS G12D (FIG. 5B) rescued mutant amplification as compared to the assay without an anti-blocker (FIG. 5A). As shown in FIGS. 5C and 5D, the addition of a mutation-specific anti-blocker for wild-type KRAS G12 (FIG. 5D) did not impair the MEOW's blocking of WT amplification as compared to the assay without an anti-blocker (FIG. 5C).

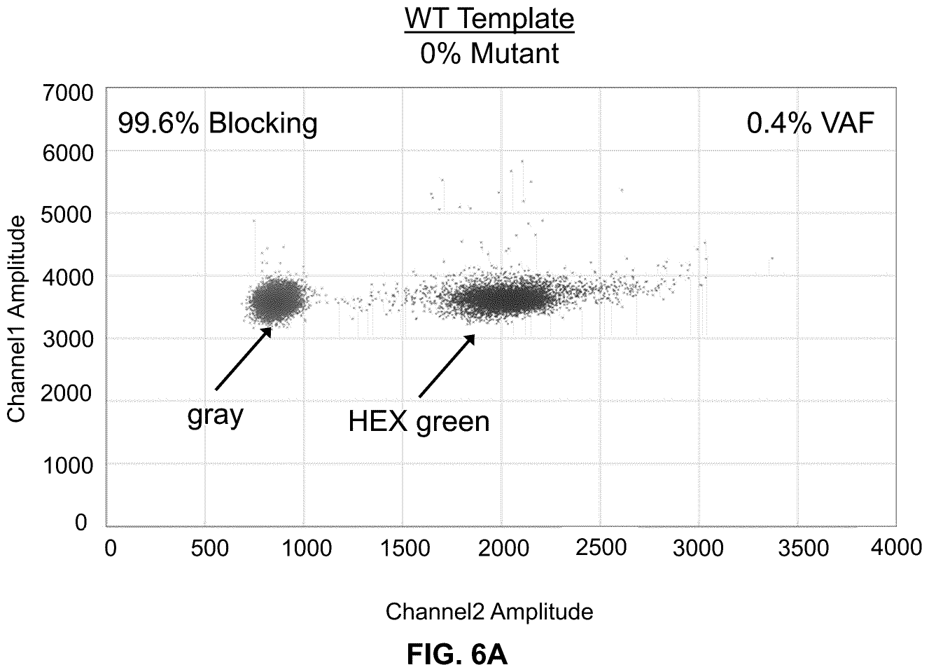

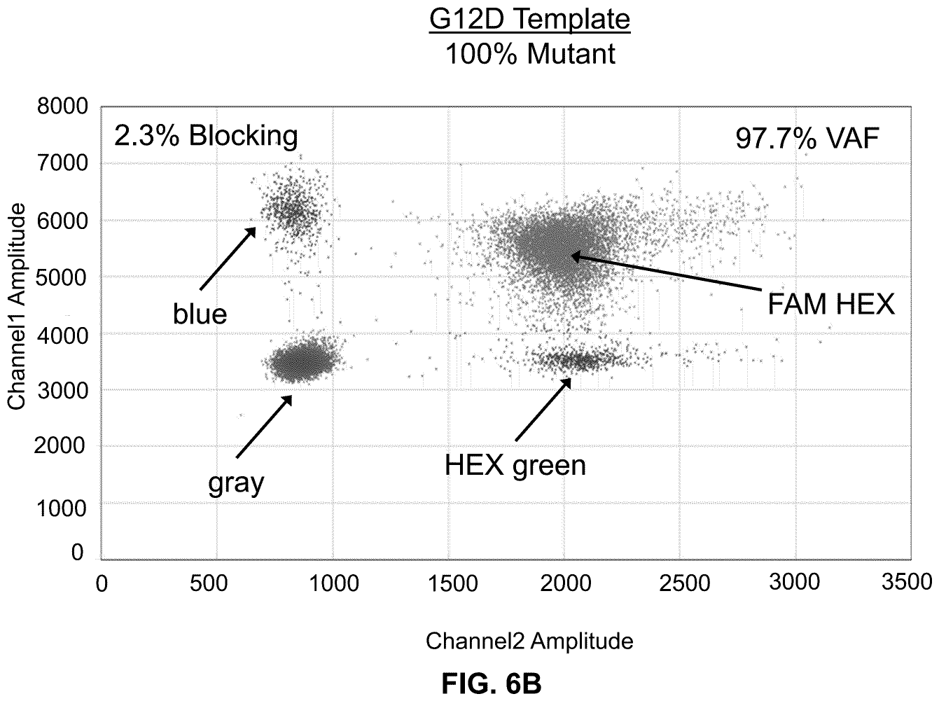

This MEOW technology is relevant to, for example, both real time and digital PCR technologies (FIGS. 6A-6B). In the presence of a MEOW, KRAS-G12 template is not detected with hypervariable assay (FAM positive droplets) (FIG. 6A, showing no “blue” signal). The MEOWs of the instant invention, in contrast, only allow mutated KRAS template, such as KRAS-G12D, to be detected with the hypervariable assay resulting in FAM single positive (indicated as “blue” in FIG. 6B) and FAM-HEX double positive droplets (indicated as “FAM HEX” in FIG. 6B), when duplexed with the universal KRAS assay. The concentration of FAM positives divided by HEX positives for WT and G12D template results in 0% and 97% mutant respectively, suggesting a 97% efficient blocking of WT.

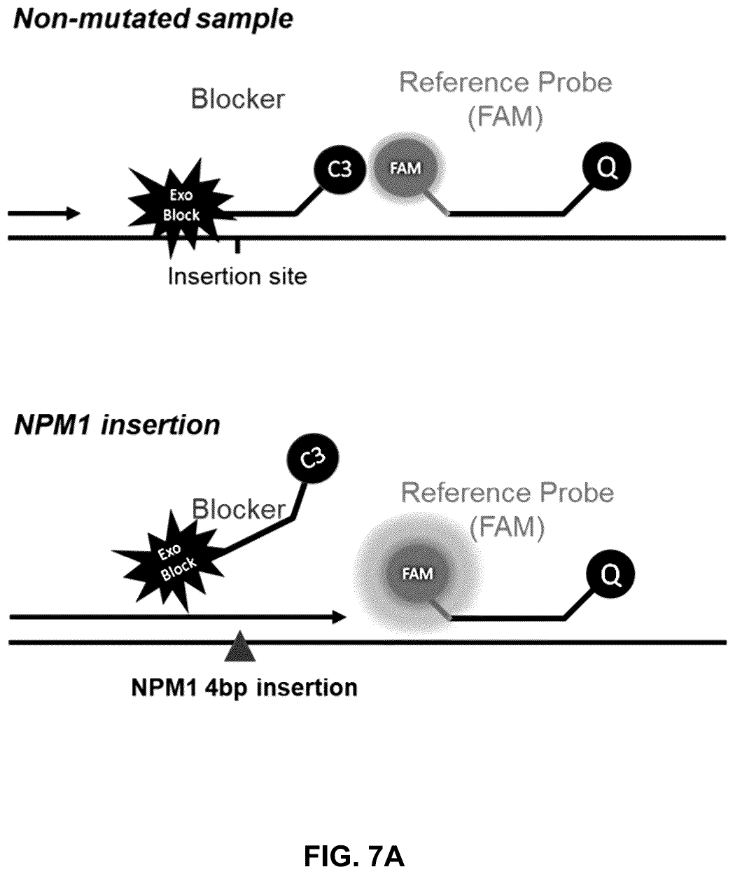

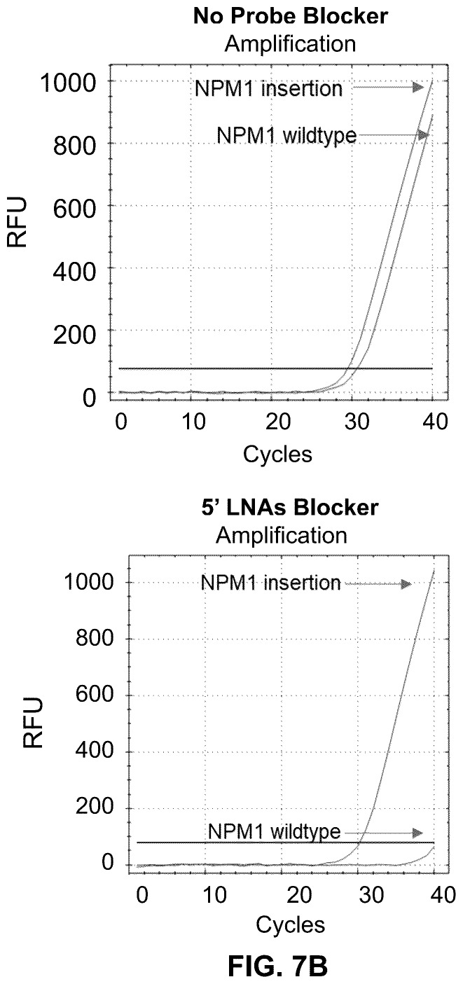

The MEOW technology described herein is applicable for a range of applications and mutation types. For example, they are applicable for different genes and different types of mutations, including insertion mutations. NPM1 is a gene commonly mutated in acute myeloid leukemia. Specifically, four nucleotide insertion events occur at the genomic position corresponding to amino acid 288. We designed MEOWs to the wild type sequence at this corresponding amino acid site and show that with minor optimization, this technology is applicable beyond KRAS and SNPs (FIG. 7A-7B). FIG. 7A again summarizes the underlying mechanism of a MEOW (no amplification product for WT non-mutated sample—left panel of FIG. 7A compared to amplification for mutant because the MEOW does not hybridize and the labelled probe (referred in FIG. 1A as “Reference Probe (FAM)” is therefore readily amplified by PCR). Optical detection as a function of PCR cycle number is provided in FIG. 7B, illustrating with the probe blocker, the mutant was preferably amplified and distinguishable from WT.

### Example 2: SNP Typing

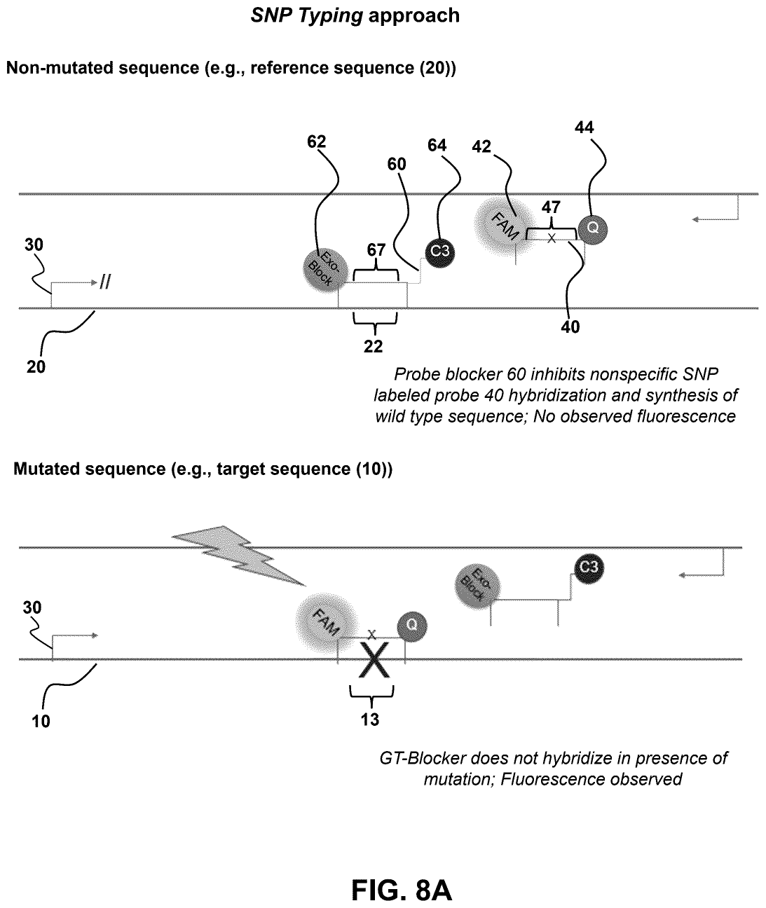

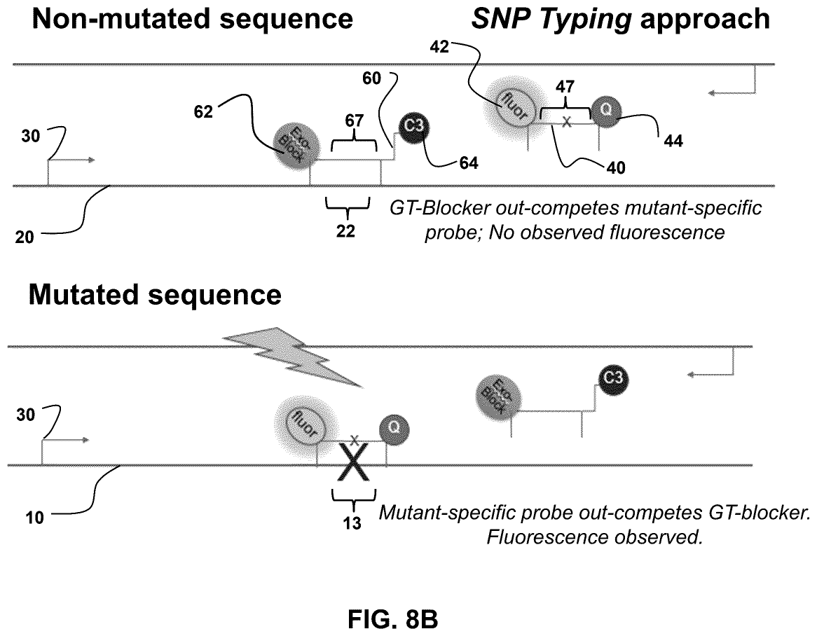

The SNP typing approach also incorporates two separate probes, including 5′ probes that target two separate amplicons, (for two putative amplicons, a non-mutated sequence (e.g., reference sequence 20) (FIG. 8A, top & FIG. 8B, top) and a mutated sequence (e.g., target sequence (10), (FIG. 8A, bottom & FIG. 8B, bottom) but utilizes additional channels beyond FAM/HEX. The first, “labelled probe” 40, corresponds to a universal amplicon and is designed to target any sequence regardless of mutation presence. The labelled probe 40 comprises a FAM 42 and a quencher 44 and a labelled probe sequence 47 configured to hybridize to a target sequence region 13 of the target sequence 10 (FIG. 8A, top). While FIG. 8A depicts a labelled probe comprising a FAM 42, any suitable fluorophore may be used (as depicted as “fluor” in FIG. 8B), such as FAM, HEX, VIC, SUN, TET, Yakima Yellow, Cy5, JUN, Cy5.5, TAMRA, ABY, ATTO550, Texas Red/ROX, ATTO590, and/or ATTO425. Any of the methods provided herein may further comprise generation of a universal amplicon that generates a universal signal that is useful as a positive control.

The first labelled probe 40 that targets any sequence regardless of mutation presence or absence is also referred to as a “universal probe” having an attendant universal signal generated by the attendant universal amplicon. The second amplicon (FIG. 8A, bottom and FIG. 8B, bottom) incorporates a MEOW 60 specific to a reference sequence (e.g., wild type KRAS G12/G13) and includes competitive, SNP-specific probes. The MEOW 60 comprises a 5′ exonuclease resister 62, a 3′ extension blocker 64 configured to prevent elongation by a polymerase, and a reference binding region 67 positioned between the 5′ exonuclease resister and the 3′ extension blocker. The reference binding region 67 is configured to hybridize to a binding reference region 22 of the reference sequence 20. In embodiments, the labelled probe 40 hybridizes to the target sequence region 13 at a higher binding affinity than the MEOW 60 non-specific binding to the target sequence region 13. The MEOW 60 hybridizes to the binding reference region 22 at a higher binding affinity than the labelled probe 40 non-specific binding to the binding reference region 22.

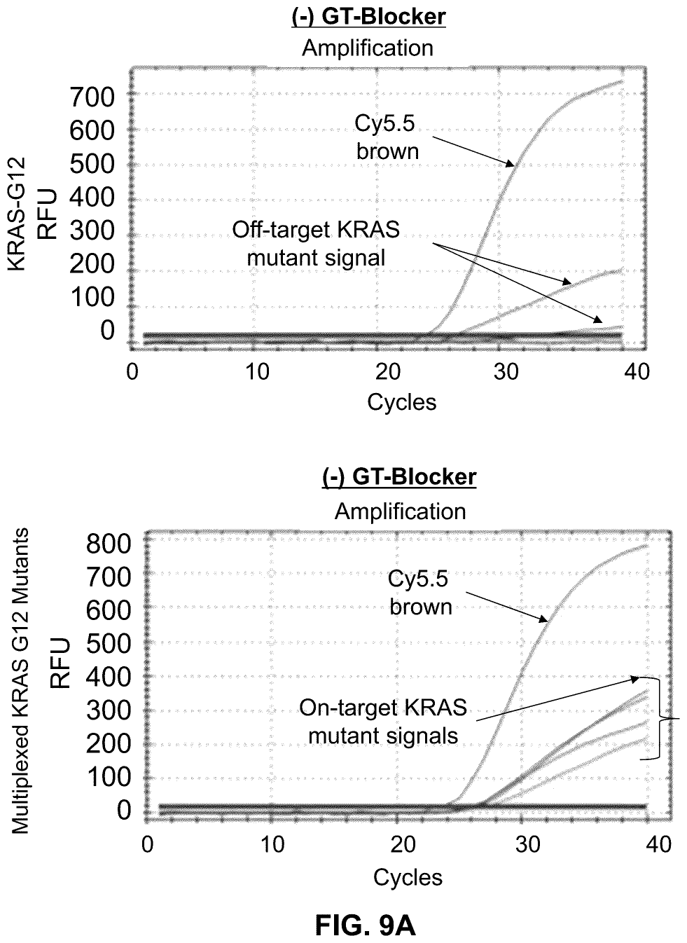

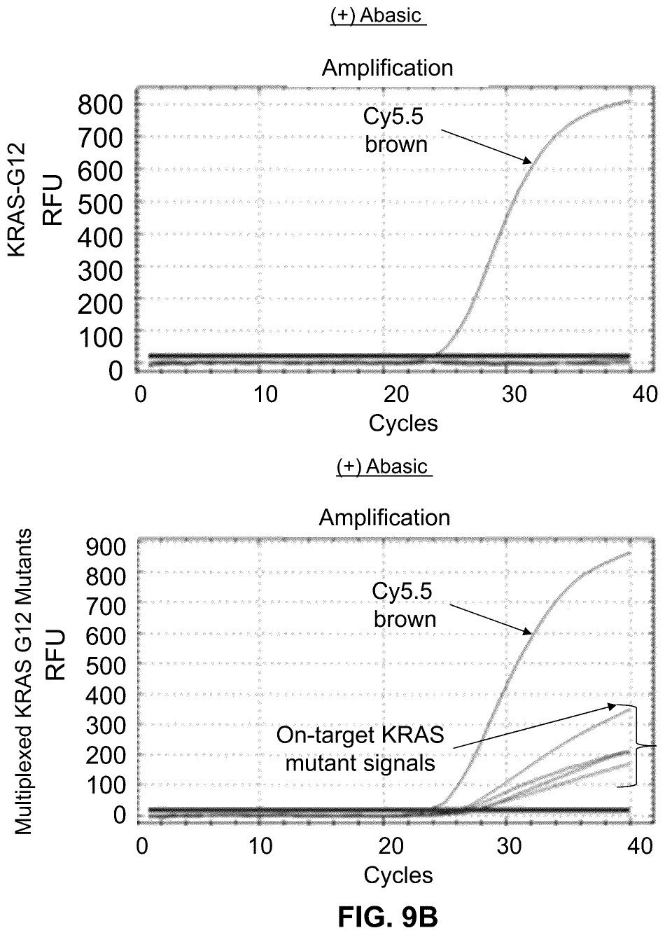

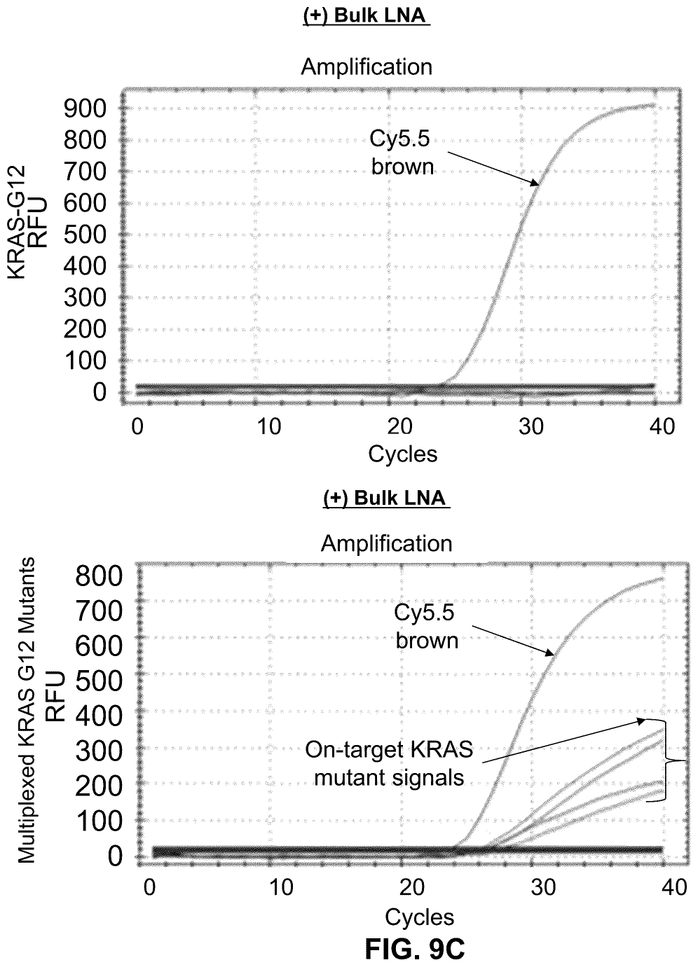

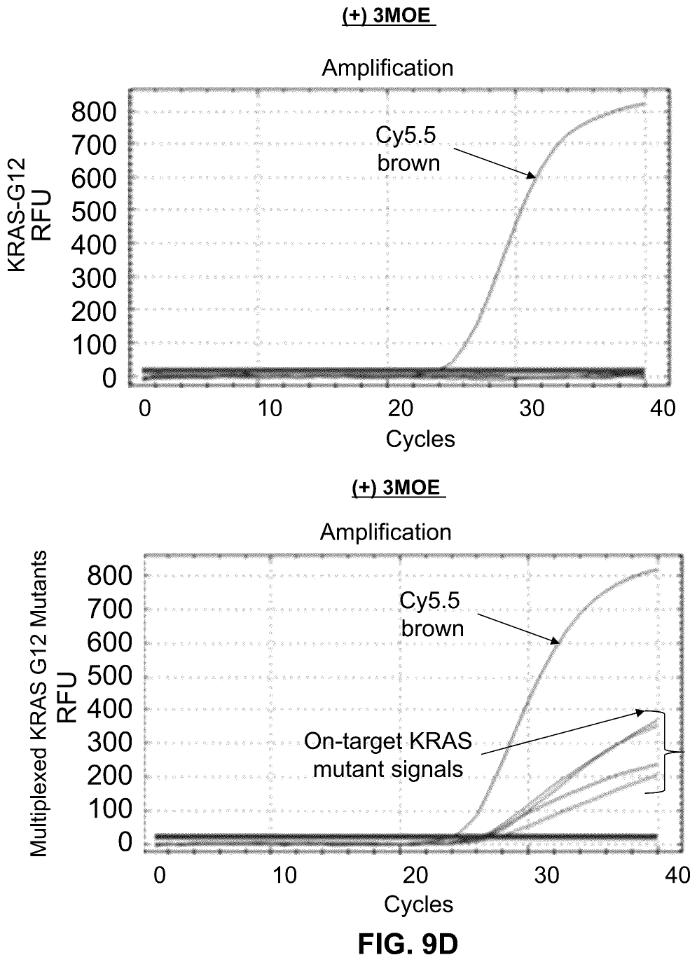

FIGS. 9A-9D outline results from our multi-reaction formulation capable of discriminating KRAS G12A, G12R, G12D, G12C, G12S, G12V, G13C, and G13D. Importantly, the MEOW enhanced specificity by preventing nonspecific probe hybridization to wild type sequence and also enhanced sensitivity by dampening the wild type KRAS G12/G13 amplicon synthesis.

Various MEOW designs were shown to inhibit nonspecific amplification of wild type sequence (FIGS. 9A-9D). When no MEOW was present, off-target amplification of KRAS mutant signal in the KRAS-G12 well was observed, as shown in FIG. 9A. When a MEOW was present, for example, SEQ ID NO: 42, off-target amplification of mutant signal in KRAS-G12 wells was not observed (FIG. 9B top panel) while on-target mutant amplification was not impacted (FIG. 9B bottom panel). The MEOW corresponding to SEQ ID NO: 41, inhibited off-target amplification of mutant signal in KRAS-G12 wells as indicated by the lack of detectable mutant signal observed (FIG. 9C top panel) while on-target mutant amplification was not impacted (FIG. 9C bottom panel). Additionally, the MEOW corresponding to SEQ ID NO: 44 inhibited off-target amplification of mutant signal in KRAS G12 wells as indicated by the lack of detectable mutant signal observed (FIG. 9D top panel) while on-target mutant amplification was not impacted (FIG. 9D bottom panel). Wells without MEOW resulted in off-target amplification of KRAS mutant signal in the KRAS-G12 well (FIG. 9A). These results suggest that MEOWs prevent off-target amplification of mutant signal in KRAS G12 wells without impacting on-target mutant amplification.

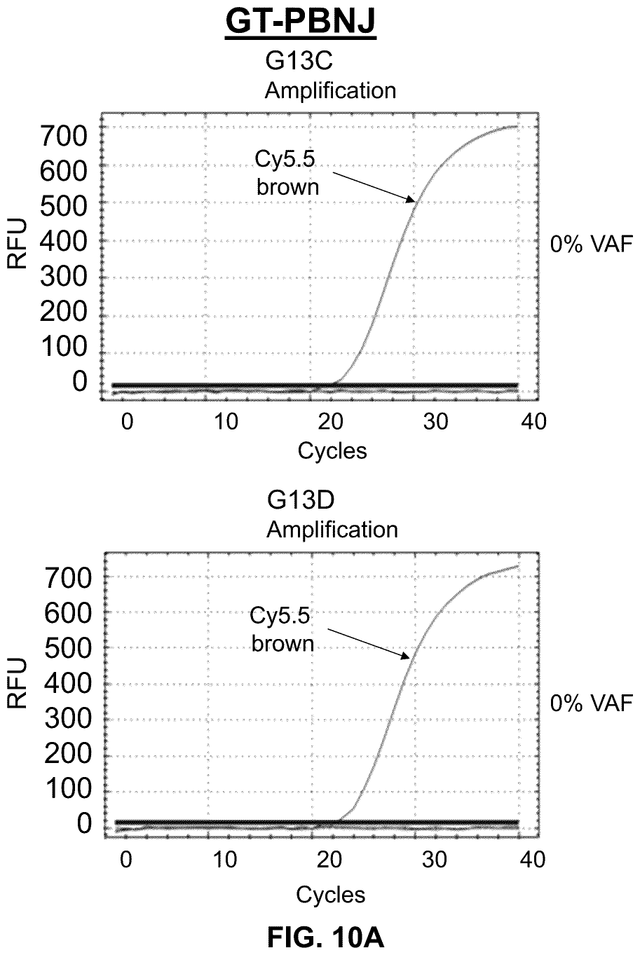

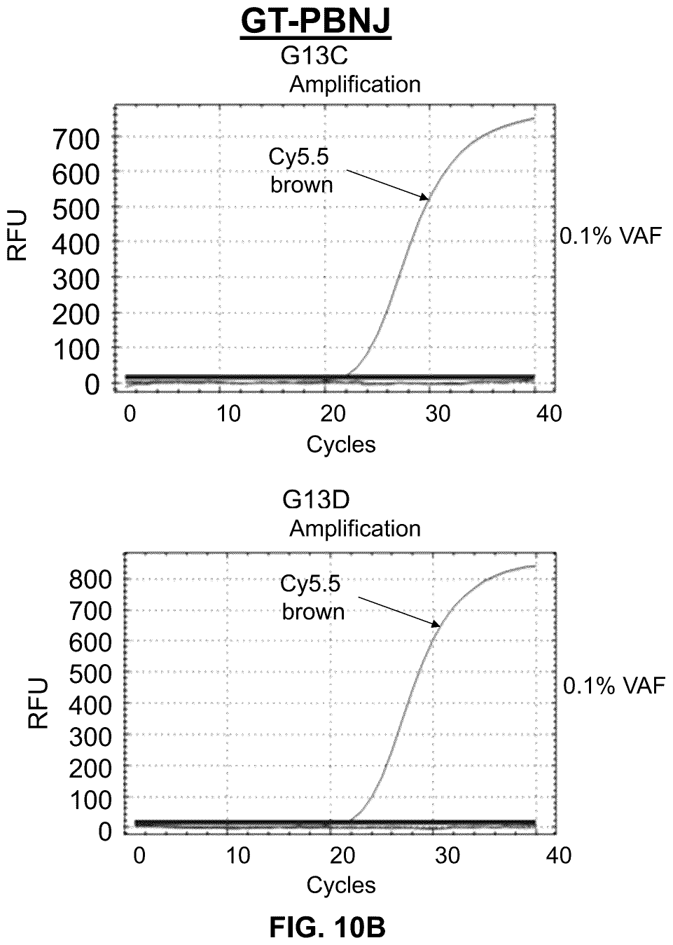

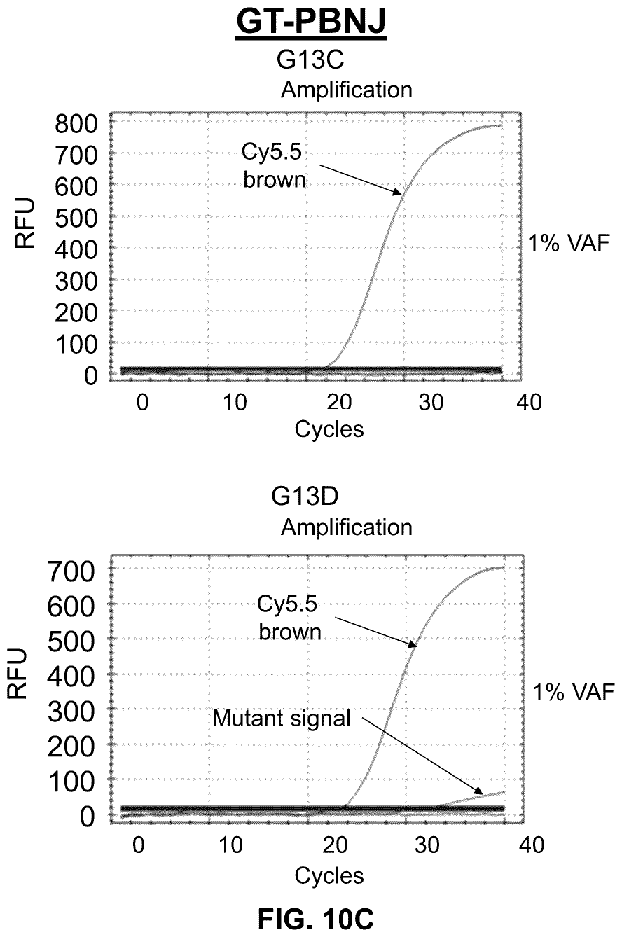

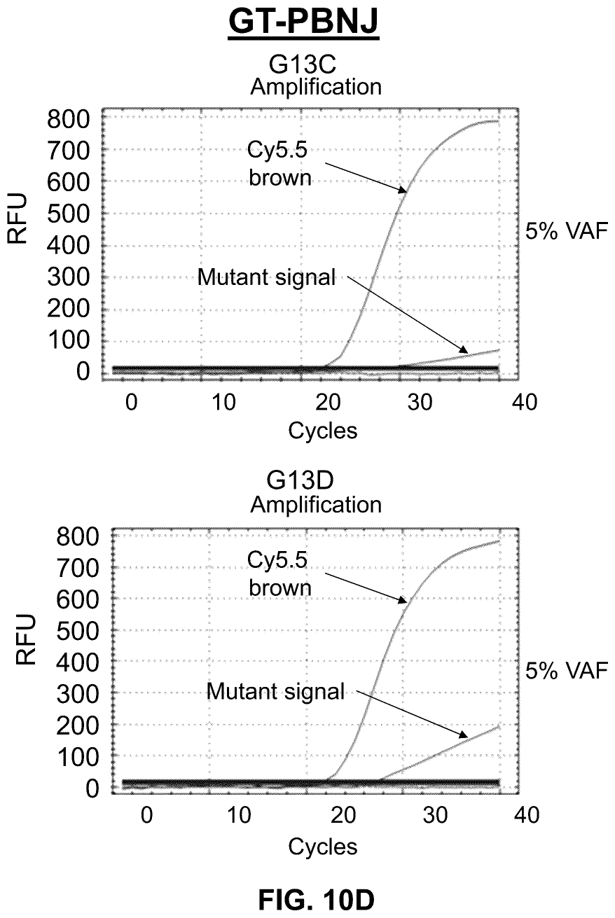

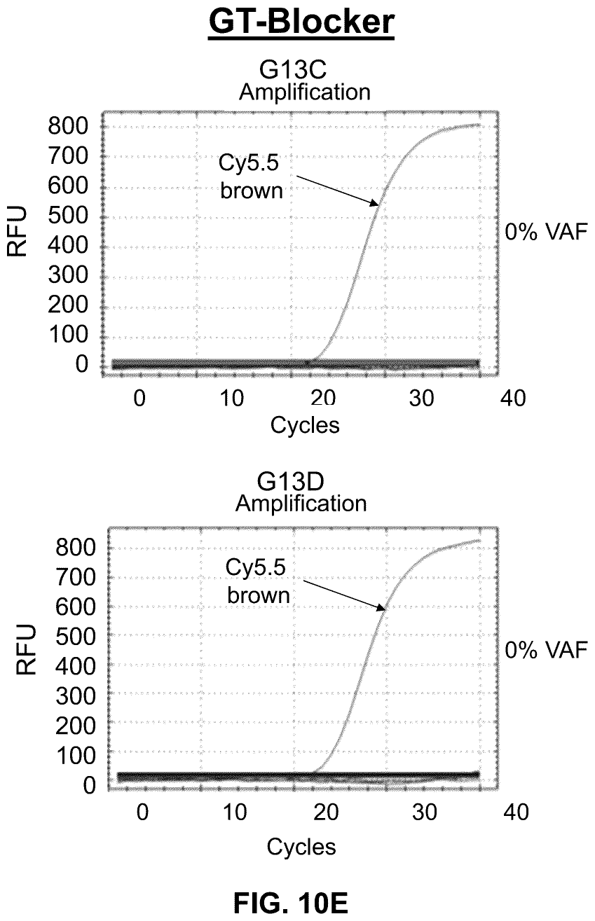

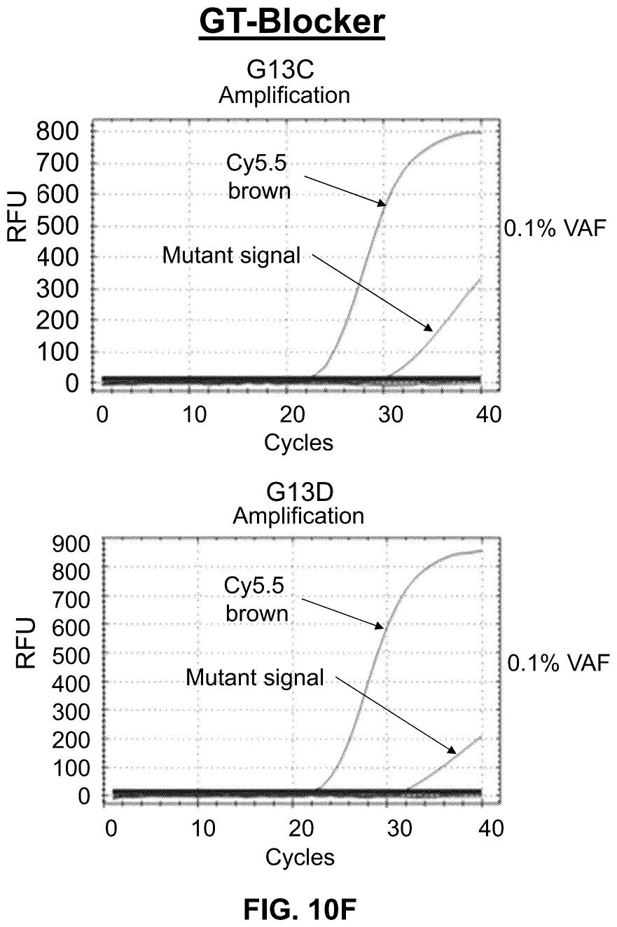

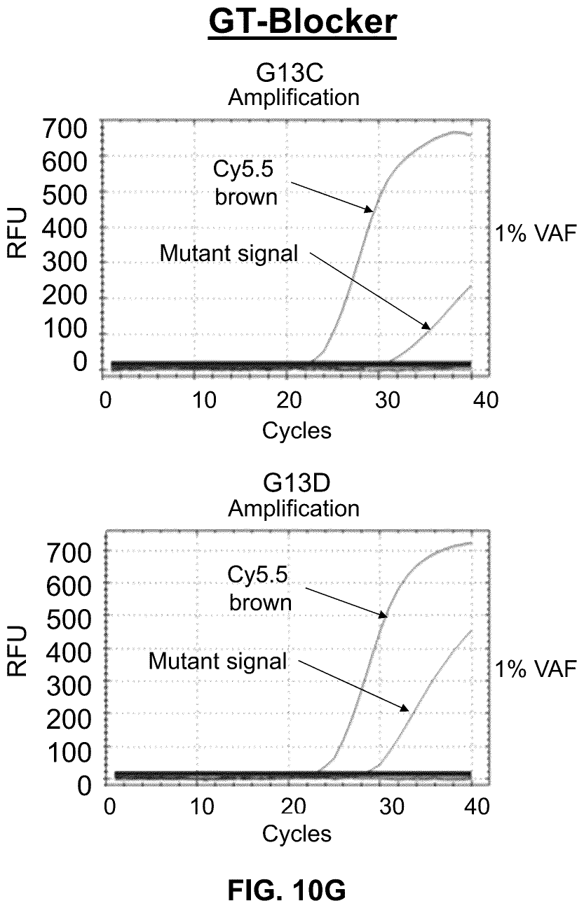

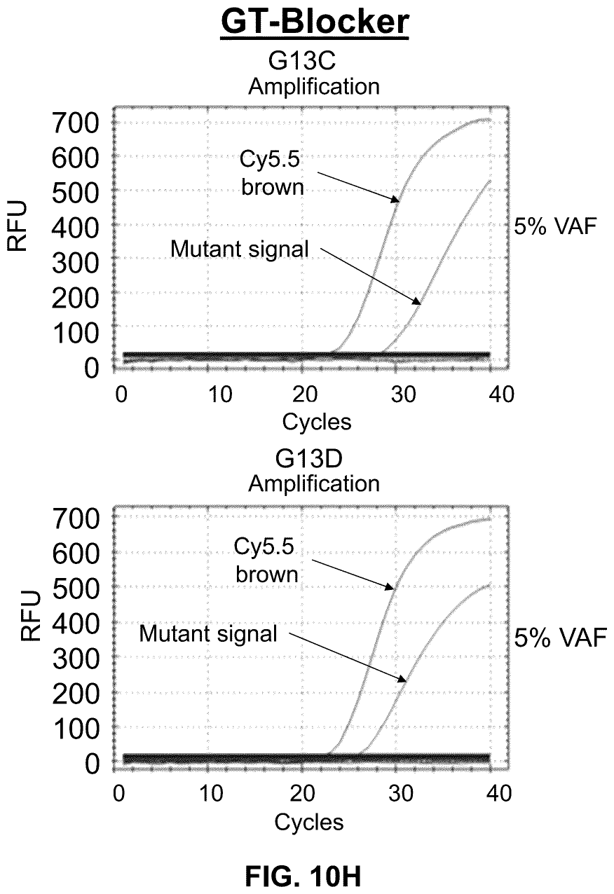

Enhanced sensitivity is achieved compared to the PBNJ technology described in U.S. Pat. Pub. No. 2023/0250467, with the instant MEOW 3′ and 5′ modification providing functional benefits around enhanced specificity. Both the PBNJ and the MEOW do not result in off-target KRAS mutant signals in wild type assays (FIG. 10A and FIG. 10E, respectively). However, the PBNJ blocker platform, while having enhanced specificity against wild type, can suffer certain sensitivity challenges. For example, we were only able to detect 5% VAF of G13C (FIG. 10D, top panel) and 1% VAF of G13D (FIG. 10C, bottom panel) with the PBNJ technology, with little to no detection of 0.1% VAF of G13C and 0.1% VAF G13D (FIG. 10B) and 1% VAF of G13C (FIG. 10C, top panel). However, MEOW resulted in the ability to detect 0.1% VAF G13C (FIG. 10F, top panel) and 0.1% VAF G13D (FIG. 10F, bottom panel), in addition to 1% VAF G13C and 1% VAF G13D (FIG. 10G) and 5% VAF G13C and 5% VAF G13D (FIG. 10H). Additional results for MEOW detection capabilities with respect to other KRAS mutations as compared to a commercial kit (QIAGEN Therascreen®) are provided in FIG. 11 (also provided in Table 7, below). These results suggest the advantages provided by the instant MEOW technology and related PCR methodology. Probe competition/typing formulations designed with MEOW technology can detect 0.1% VAF (FIGS. 10E-10H) and lower (e.g., 0.02%).

Table 7 shows a table summary demonstrating that the instant MEOW technology affords 1.6 to 64-fold increase in sensitivity compared to QIAGEN Therascreen® in vitro diagnostic reported values.

TABLE 7
Comparison of MEOW technology sensitivities and QIAGEN
Therascreen ® sensitivities.

| Target | QIAGEN Therascreen ® | GTM GT-Blocker |
|---|---|---|
| G12A | 0.8% | 0.5% |
| G12C | 1.5% | 0.5% |
| G12D | 6.4% | 0.1% |
| G12R | 2.6% | 0.5% |
| G12S | 5.7% | 0.5% |
| G12V | 1.6% | 0.5% |
| G13C | ND | 0.1% |
| G13D | 6.4% | 0.1% |

### Example 3: Exemplary Use(s) of MEOW

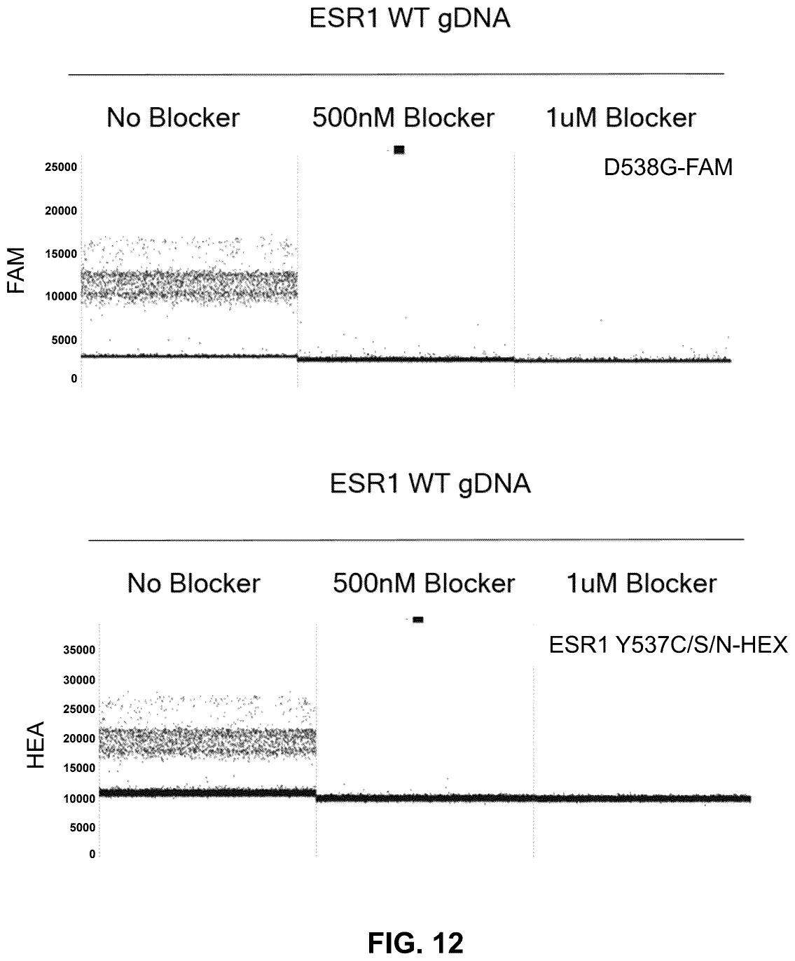

FIGS. 12-16 are experimental results illustrating the further usefulness of MEOW(s).

FIG. 12 demonstrates the utility of the MEOW in digital PCR in preventing non-specific amplification of wild-type DNA. A multiplexed typing assay targeting common SNVs in ESR1 was conducted, the MEOW was designed to be specific to the wild-type/reference sequence, and labeled probes for the following mutations were included: D538G, Y537C, Y537S, Y537N, L536H, L536R, L536P, S463P, E380Q. Results from the multiplexed typing assay targeting common SNVs in ESR1 (i.e., D538G (FIG. 12, top) and Y537C/S/N (FIG. 12, bottom) depicted non-specific amplification of non-mutated ESR1 DNA even when there was no mutated DNA in the reaction (see left-most panels of FIG. 12, “No Blocker”). In contrast, the MEOW (depicted as “Blocker” in middle and right panels of FIG. 12) prevented amplification of non-mutated DNA, allowing the ESR1 MT (mutant) signal in multiple channels to be specific to their intended targets. These results suggest that the addition of one MEOW prevents cross reactivity with the wild-type sequence caused by the D538G, Y537C, Y537S, and Y537N probes.

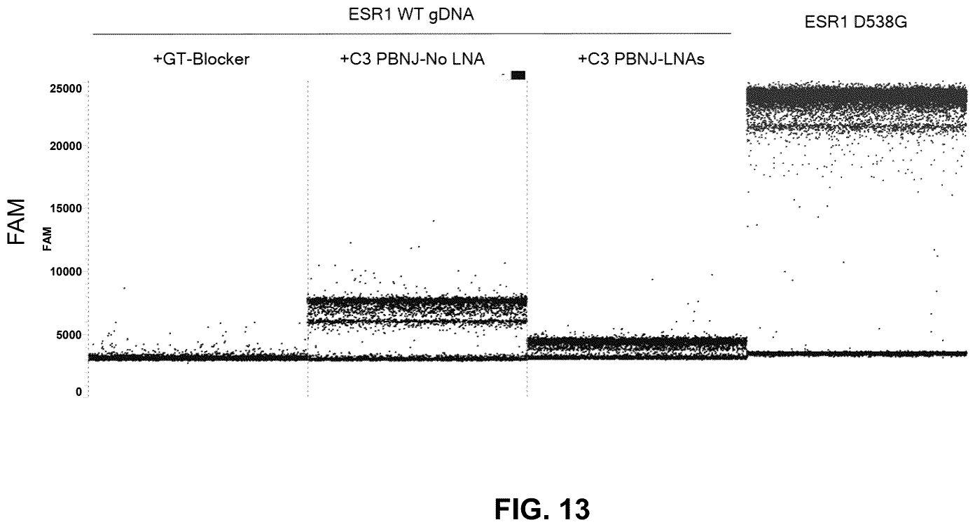

FIG. 13 demonstrates improved performance of the MEOW compared to the traditional PBNJ in preventing non-specific amplification of wild-type DNA in digital PCR. A multiplexed assay targeting common SNVs in ESR1 was conducted, both the MEOW and PBNJ were designed to be specific to the wild-type/reference sequence, and labeled probes for the following mutations were included: D538G, Y537C, Y537S, Y537N, L536H, L536R, L536P S463P, E380Q. The same concentrations of MEOW and PBNJ were administered in the assay in order to provide a direct comparison. Results from the multiplexed assay targeting common SNVs in ESR1 (i.e., D538G) depicted non-specific amplification of non-mutated DNA in the reaction in the presence of a PBNJ (600 nM) with or without an LNA at the SNV site (FIG. 13, depicted as “+C3 PBNJ-No LNA” and “+C3 PBNJ-LNAs”, respectively), albeit at a lower amplitude compared to on-target amplification (FIG. 13, depicted as “ESR1 D538G”). In contrast, the MEOW (600 nM) (FIG. 13, depicted as “+GT-blocker”) substantially eliminated off target population amplification. These results suggest that MEOW was more efficient at preventing non-specific amplification of wild-type DNA.

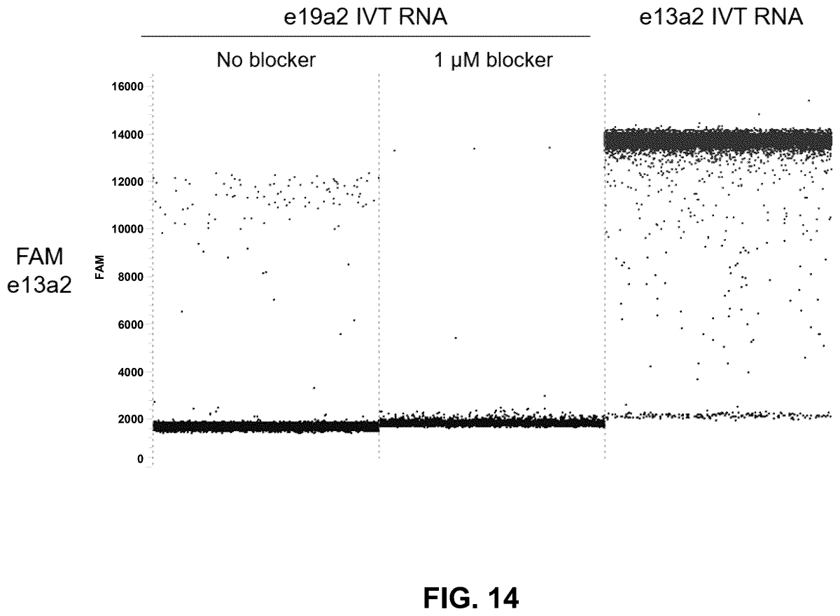

FIG. 14 demonstrates the utility of a MEOW in digital PCR in preventing undesired signals from upstream probes in a multi-target assay. A multiplexed assay was conducted targeting multiple BCR-ABL1 fusion types against RNA containing the e19a2 fusion type (exon 19 of BCR fused to exon 2 of ABL). BCR and ABL are two genes commonly fused together in chronic myeloid leukemia. BCR is “breakpoint cluster region” and ABL1 is ABL proto-oncogene 1. The e19a2 fusion type was tested in the first two panels of FIG. 14 (“No blocker” and “1 μM”), and e13a2 RNA in the last panel (“e13a2 IVT RNA”). FIG. 14 shows FAM signal, which depicts e13a2 fusion. The multiplexed assay included a primer and probe set on the following exons of BCR: Exon 1, Exon 6, Exon 13, and Exon 19 (different colors (FAM, Cy5, ROX, etc) for each). There was a reverse primer on Exon 3 of ABL.

As background, in an ela2 BCR-ABL fusion, Exon 1 of BCR is fused to Exon 2 of ABL, thus producing an amplicon between the BCR Exon1 primer/probe set with the reverse primer on ABL Exon 3. The binding sites for the primer/probes of Exon 6, 13, and 19 of BCR are not present in this fusion type. On the other hand, in the e19a2 fusion type (Exon 19 of BCR fused to Exon 2 of ABL), the primer/probe binding sites of Exon 1, 6, and 13 of BCR are present upstream of the Exon 19 fusion site. Therefore, there is a chance that large amplicons will be made (for example, between primer/probe of Exon 13 BCR with reverse primer of Exon 3 ABL).

However, results from this assay, depicted in FIG. 14, suggest that the MEOWs prevented the RNA polymerase from transcribing past the MEOW, and the exon 13, exon 6, and exon 1 cDNA binding sites were not present, and thus, only one signal was generated. Specifically, the results depicted undesired amplification coming from an upstream primer and FAM probe on exon 13 of BCR without MEOW (FIG. 14, depicted as “No blocker”). This signal was significantly reduced on e19a2 RNA template with the addition of a MEOW (1 μM) positioned between exon 12 and exon 19 (FIG. 14, depicted as “1 μM blocker”), presumably due to a block in synthesis of the transcript beyond the MEOW. On-target FAM amplification on e13a2 RNA was not quantifiably affected by the MEOW on e13a2 RNA template (FIG. 14, depicted as “e13a2 IVT RNA”).

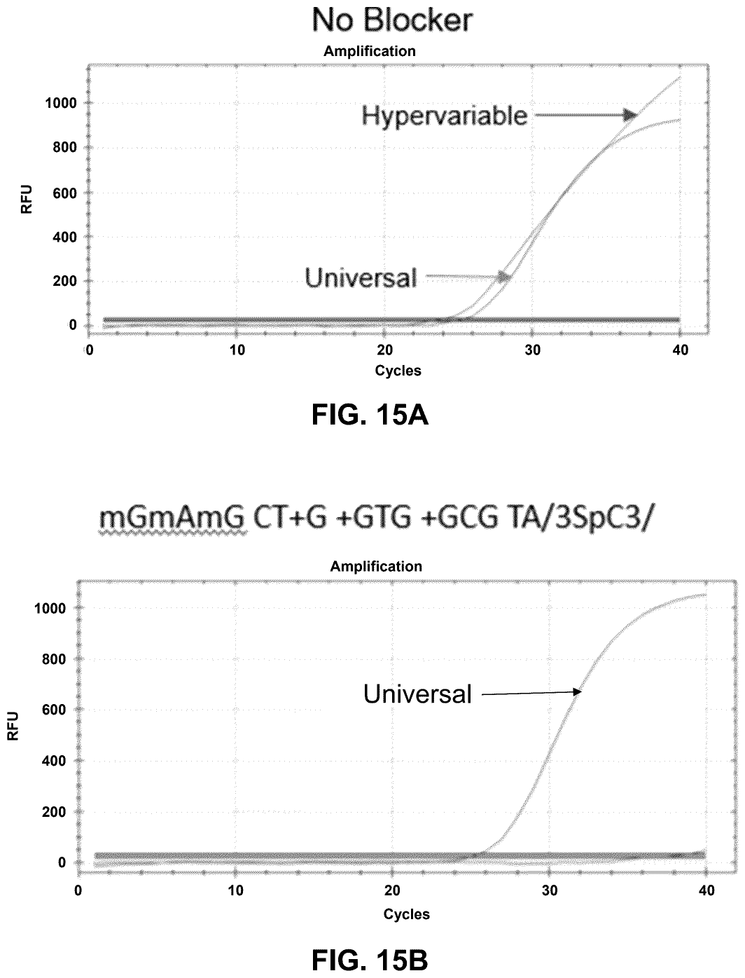

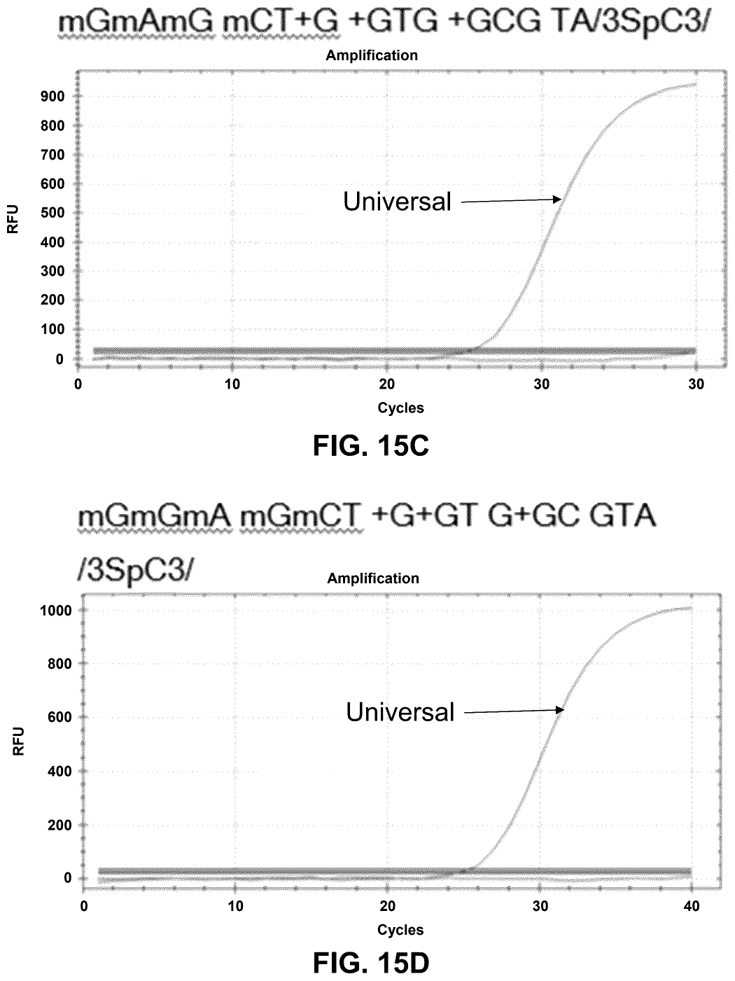

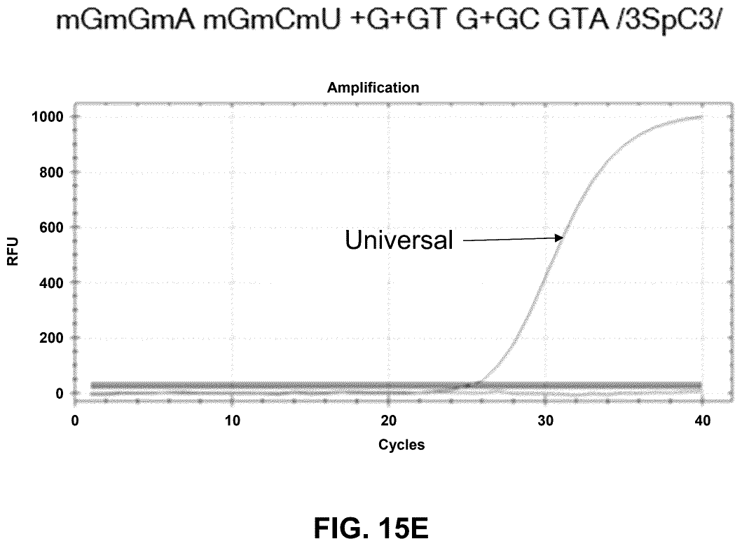

Hybrid MEOW (GT-Blockers) containing both 2′-O-Methyl RNA bases and traditional DNA bases, specific to the wild-type DNA sequence at the G12 or G13 positions of the human KRAS gene, were also generated to conduct a screening approach assay. The assay had a “universal” probe designed to show signal independent of a mutation at the G12 or G13 positions. The universal probe was labeled with HEX and is indicated by “Universal” in each of FIGS. 15A-15E. A second probe (FAM, depicted as “Hypervariable” in FIG. 15A) was designed to bind downstream to the MEOW binding site to show signal when there is a mutation present in the MEOW binding region.

The hybrid MEOW combination offered a new approach to the MEOW design, and it resulted in blocking of non-mutated KRAS DNA at the G12 and G13 codons at lower temperatures (i.e., 55° C. annealing/extension temperature) than alternative modifications (see, FIGS. 15A-15E). Wells without the hybrid MEOW showed both mutant-independent amplification of the universal target (HEX) and mutant-dependent amplification of the hypervariable target (FAM) (FIG. 15A). In contrast, addition of MEOW (GT Blockers) (1 μM) with the 2′-O-Methyl RNA bases at the 5′ end prevented amplification of the non-mutated sequence, resulting in suppression of the FAM (blue) signal when only non-mutated DNA was present (FIGS. 15B-15E). Several MEOW (GT Blockers) were tested including SEQ ID NO: 58 (FIG. 15B), SEQ ID NO: 59 (FIG. 15C), SEQ ID NO: 60 (FIG. 15D), and SEQ ID NO: 61 (FIG. 15E). These results suggest that the 2′-O-Methyl RNA bases support enhanced duplex stability, which is believed to have facilitated the substantially reduced amplification of the wild-type sequence.

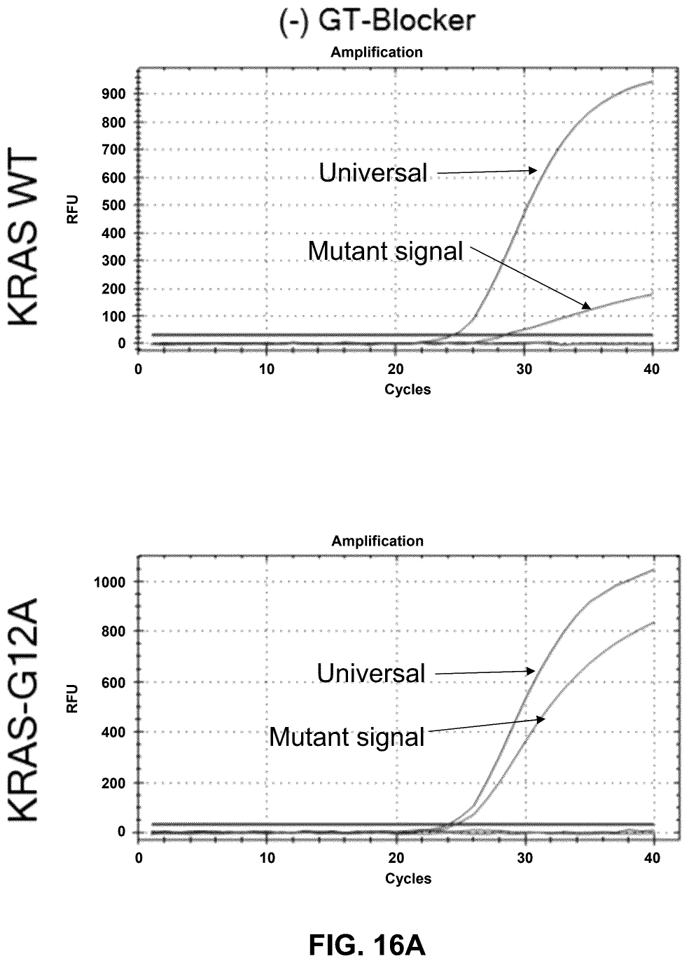

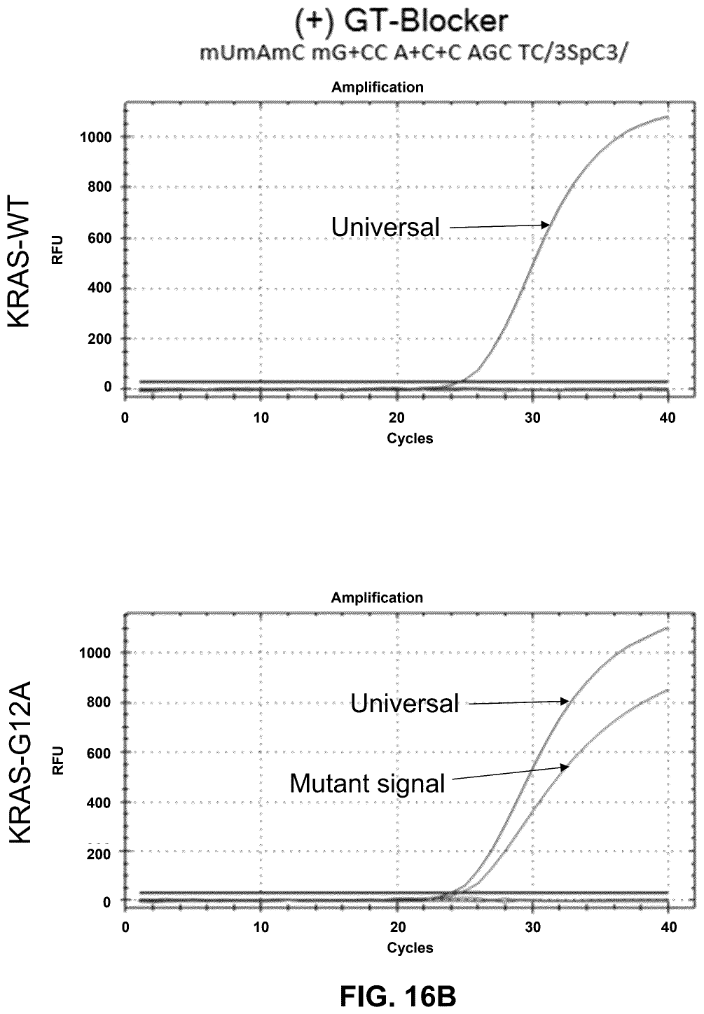

The hybrid MEOWs containing both 2′-O-Methyl RNA bases and traditional DNA bases were also tested for their applicability in typing assay configurations. A multiplexed assay targeting KRAS G12A, G12R, and G12D mutations was conducted. As depicted in FIGS. 16A-16B, mutant signal was only detected when the mutant template was present. Without the MEOW, there was mutant signal in wells containing only non-mutated KRAS DNA (FIG. 16A). With the MEOW, SEQ ID NO: 63, (500 nM), only the “Universal” signal was present in wells with non-mutated DNA present (FIG. 16B).

### Example 4: Combination of MEOW with C3-PBNJ

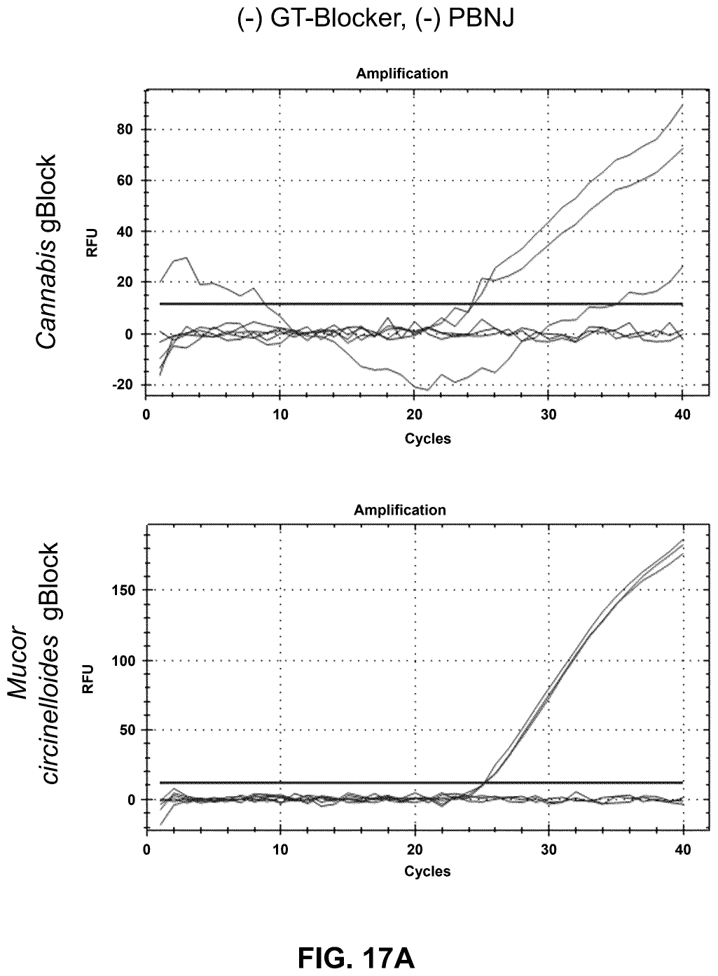

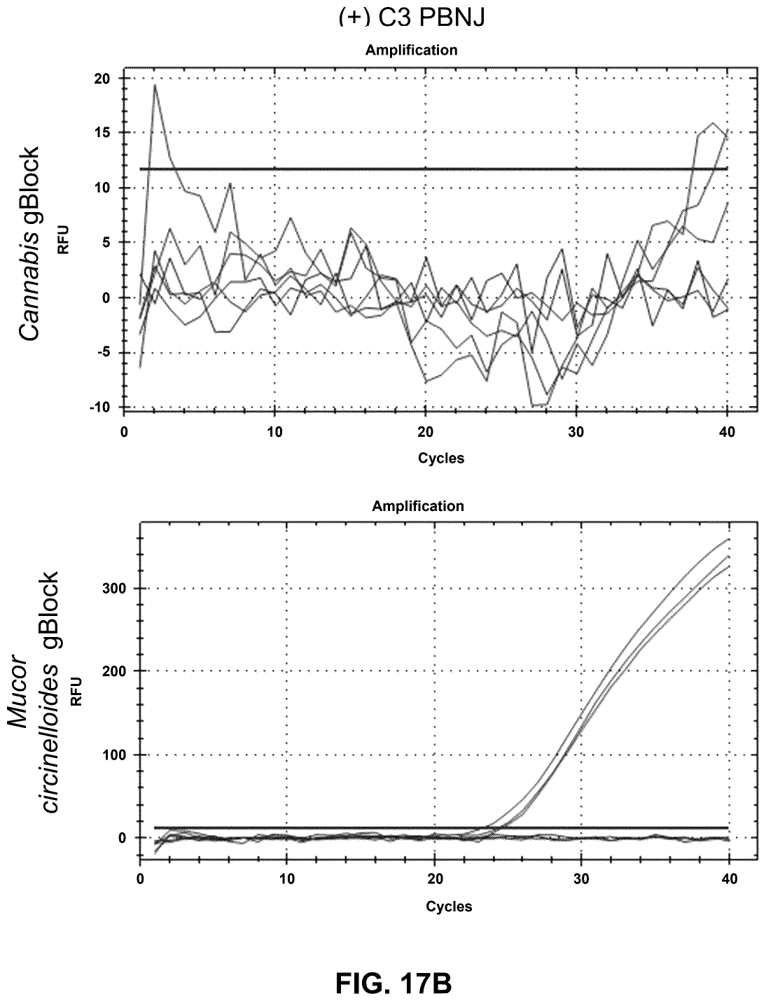

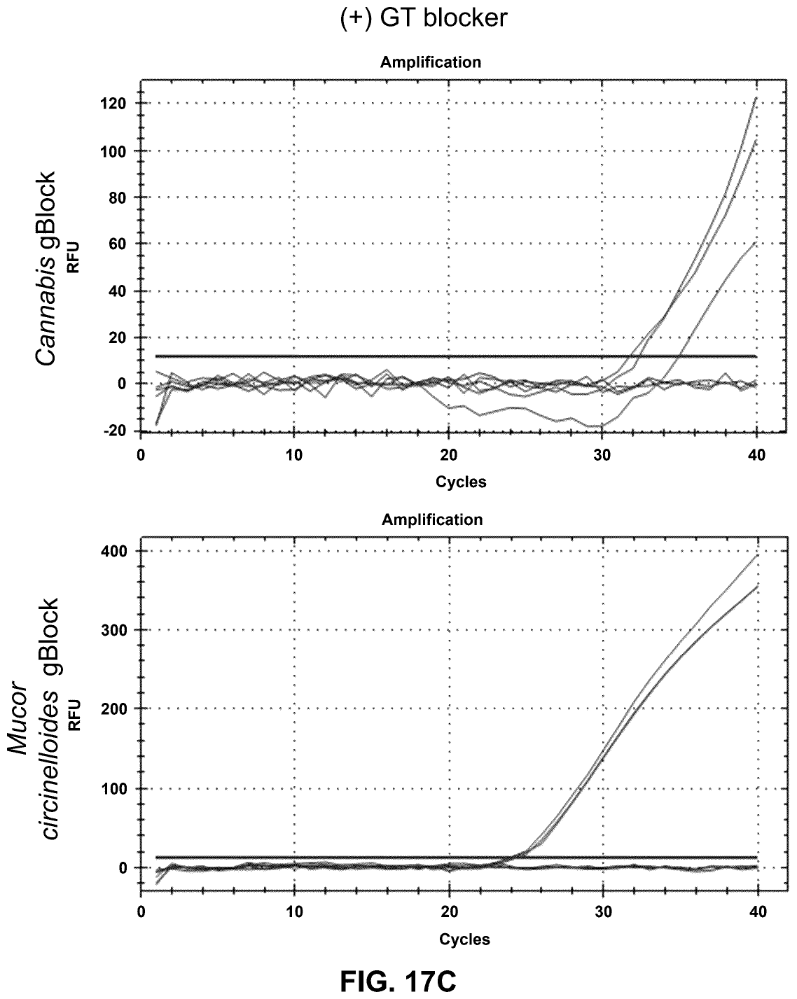

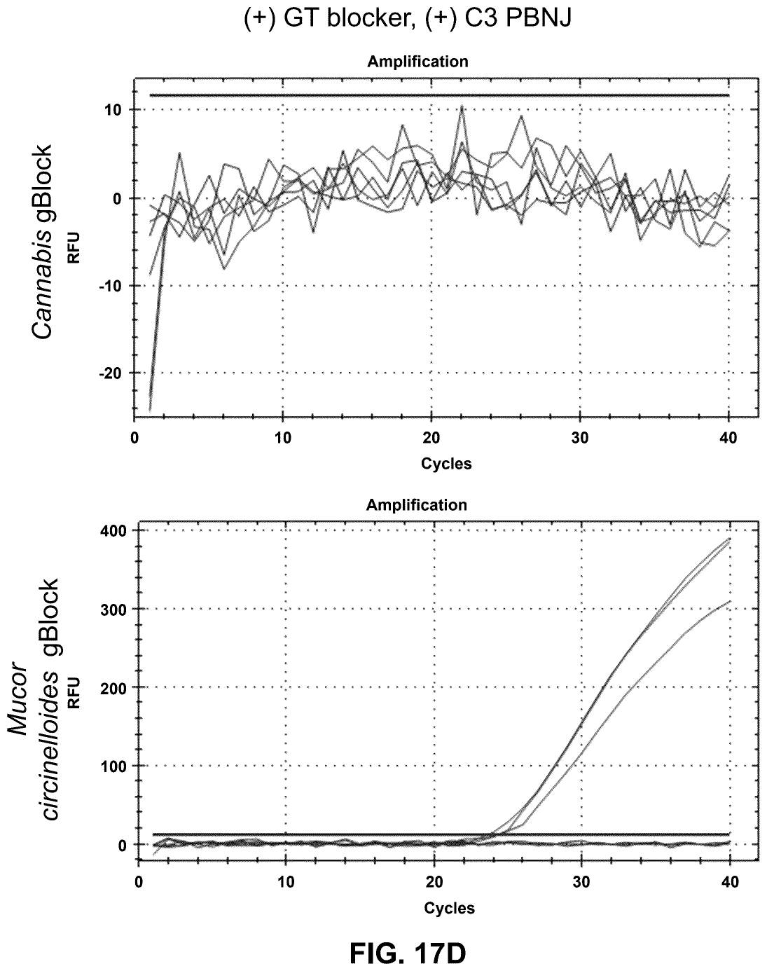

A multiplexed assay targeting total yeast and mold (TYM) for routine cannabis testing demonstrated optimal specificity with the use of a GT-blocker in combination with a C3 PBNJ. Non-specific Mucor circinelloides signal was produced on synthetic cannabis DNA (FIG. 17A) in the absence of a MEOW or C3 PBNJ. Addition of a C3 PBNJ or GT-blocker individually reduced but did not eliminate the non-specific signal (FIG. 17B and FIG. 17C). No signal from synthetic cannabis DNA was produced when adding both a C3 PBNJ and GT-blocker to the assay compared to the assay with no blocker/C3 PBNJ and the C3 BPNJ and blocker individually (FIG. 17D). Additionally, the GT-blocker and C3 PBNJ in combination did not impact on-target Mucor circinelloides amplification (FIG. 17D). These results further highlight the utilities of MEOW technology in a broad range of applications.

### Example 5: Additional Designs for MEOWs (GT-Blockers) Include 2′ Amino, 2′ Fluro, or Hairpin Modifications

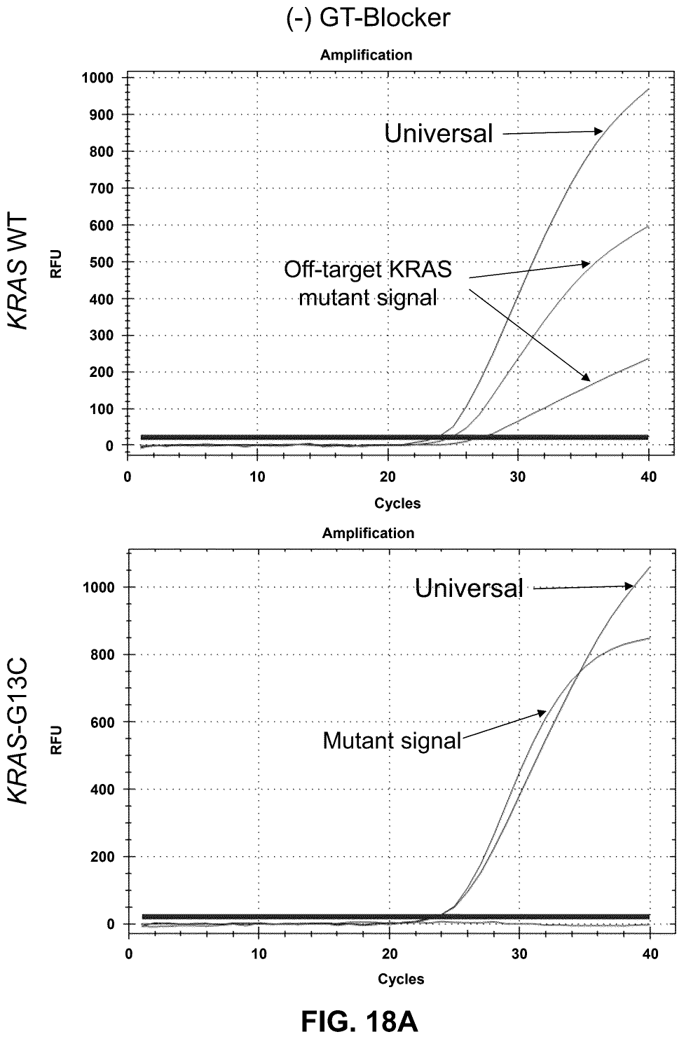

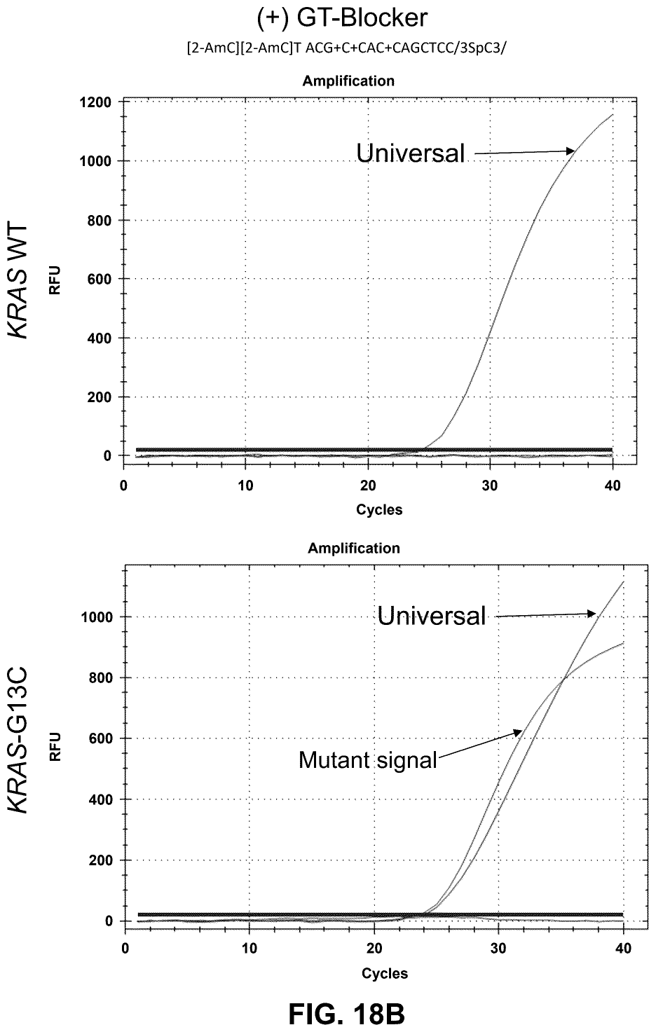

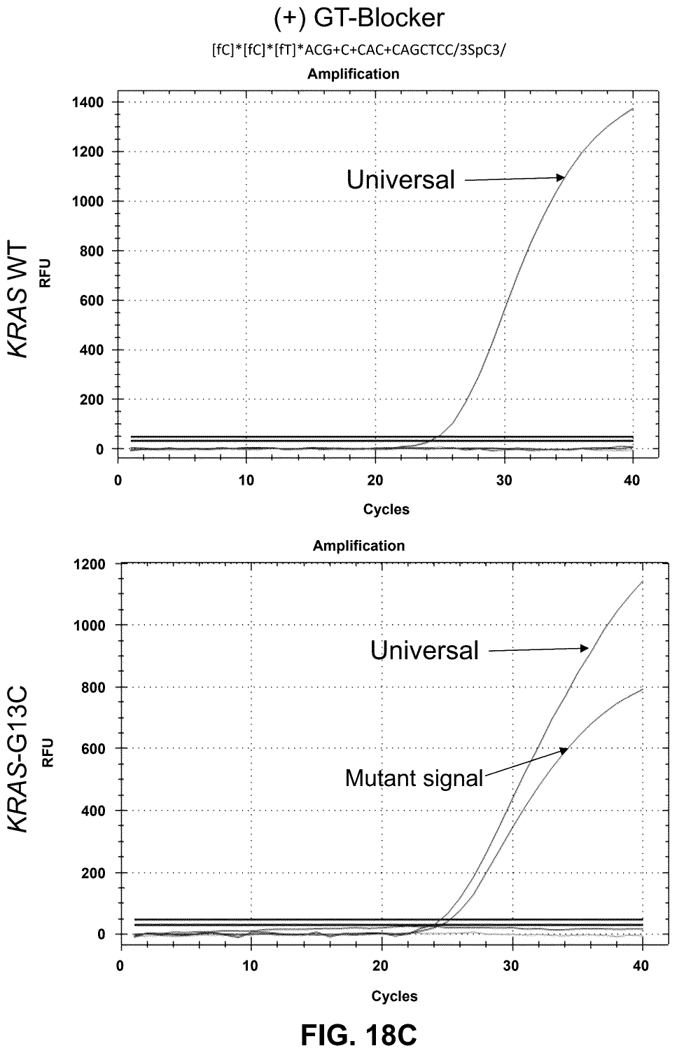

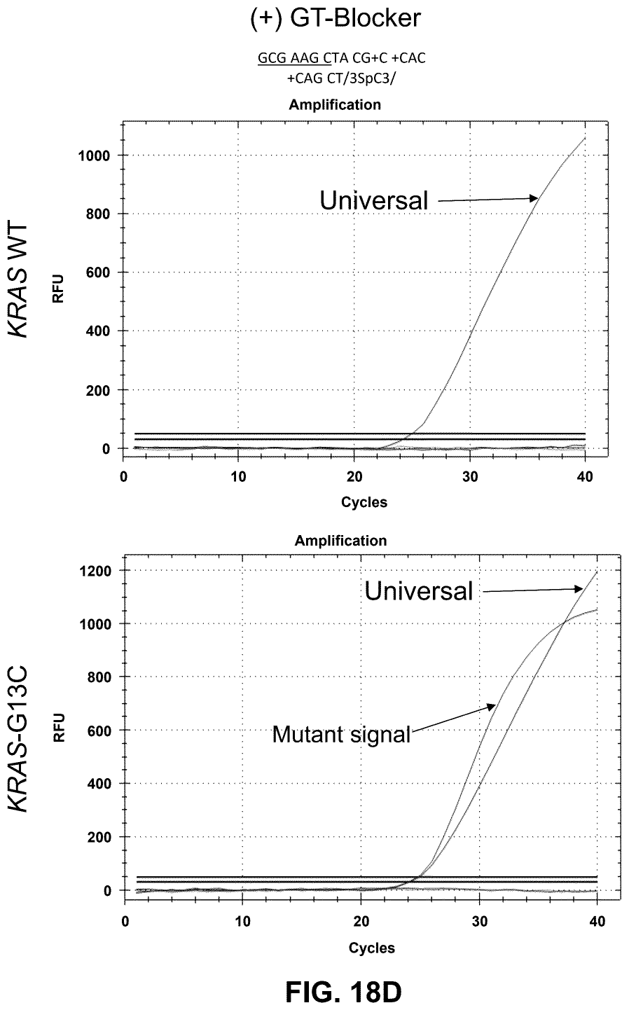

A multiplexed assay targeting KRAS G13C and G13D mutations showed mutant signal when only non-mutant template was present as shown in FIG. 18A. Without the GT-Blocker there was mutant signal in wells containing only non-mutated KRAS DNA (FIG. 18A). With the GT-Blocker only the “Universal” signal was present in non-mutated DNA wells. For example, only “Universal” signal was observed in non-mutated DNA wells as shown in FIG. 18B (top panel) when SEQ ID NO: 149 corresponding to a 2′ Amino MEOW was present. Further, when SEQ ID NO: 149 was present, on-target amplification was not impacted (FIG. 18B, bottom panel). When SEQ ID NO: 150, corresponding to 2′ Fluro MEOW was present, again, only “Universal” signal was observed in non-mutated DNA wells as shown in FIG. 18C (top panel). Further, when SEQ ID NO: 150 was present, on-target amplification of mutated DNA was observed (FIG. 18C, bottom panel). When SEQ ID NO: 151, corresponding to hairpin modifications MEOW was present, only “Universal” signal was observed in non-mutated DNA wells as shown in FIG. 18D (top panel). Additionally, when SEQ ID NO: 151 was present, on-target amplification of mutated DNA was observed (FIG. 18D, bottom panel).

### Example 6: Ultra-Sensitive RT-PCR Multiplex Panel for Eight KRAS G12-13 Mutations

As described, the methods, systems and kits provided herein are useful as ultra-sensitive PCR assays for cancer research, pathogen detection, and wastewater-based epidemiology testing. This example is a cancer molecular assay, specifically a multi-target RT-PCR assay kit featuring robust quality control metrics and internal process controls. The kit is a Real-Time KRAS G12-G13 Typing PCR kit that targets common mutations found in many human cancers. This assay kit is useful with a range of Real-Time PCR systems, including but not limited to the Bio-Rad CFX96 Touch™ Real-Time PCR system. Provided is ultra-sensitive detection of eight key KRAS mutations on a Real-Time PCR platform.

KRAS mutations are one of the most commonly occurring gene mutations associated with several different cancers such as lung, colorectal, and pancreatic cancers. Detection of these mutations has been used to direct treatment options for improved patient outcomes. While genetic sequencing is an option, using faster, more targeted detection methods such digital and RT-PCR saves time and money, especially when monitoring treatment response over time. Provided herein is an ultra-sensitive, patent-pending RT-PCR assay that quickly detects as low as 0.1% VAF KRAS mutations, providing improved detection limits from similar assays on the market today, with a comparable sensitivity to digital PCR.

The panel is particularly useful in that there is: (1) Broad coverage—Discriminates eight relevant mutations found in the human KRAS gene: G12C, G12V, G12S, G12A, G12R, G12D, G13C, and G13D. (2) Ultra-sensitive, low input DNA assay that can achieve ≤0.03% variant allele frequency (VAF) across all mutations for improved detection limits compared with similar conventional assays on the market—even compared to digital PCR assays. (3) Economical and fast, with results in a few hours. (4) Compatibility with a range of samples, including but not limited to formalin-fixed paraffin-embedded (FFPE), liquid biopsy, fresh/frozen tissue, cell-free DNA (cfDNA), circulating tumor (ctDNA), and low-yield samples, and plasma samples. Furthermore, the claimed MEOW chemistry is robust, with applications including tests related to SARS-CoV-2, variants, and multiple pathogens including influenza A/B and H5N1, RSV, C. auris, polio, and others. All these applications are provided with digital PCR sensitivity in RT-PCR.

The validated kit is provided with all primers, probes and controls to provide high throughput with multiplexing and optimized workflow for faster throughput and lower reagent cost (multiplexed). There is a high sensitivity, with detection of ≤0.1% variant allele frequency (VAF) mutant DNA from wild-type targeted DNA mutations. The methods are sample ready, in that they are compatible with a range of samples, including FFPE, liquid biopsy, fresh/frozen tissue, cell-free DNA (cfDNA), circulating tumor (ctDNA), and low-yield samples.

The KRAS multiplexed kit covers the G12C mutation and a non-mutated region of the KRAS gene. KRAS G12C and non-mutant KRAS are included as qualitative positive controls. PCR Kits available in both Real Time PCR (RT-PCR) and Digital PCR formats. Includes primers, probes and precisely measured and qualified positive and negative controls.

Representative sequences include, but are not limited to those listed in TABLEs 1A-1C (wherein the symbol+refers to a LNA position):

TABLE 1
Representative Sequences for KRAS mutation multiplex detection

| SEQ ID | | |
|---|---|---|
| NO: | Oligo Name | Oligo Sequence |
| 117 | Universal-KRAS Forward Primer 1 | CCC ATG ACA CAA TCC AGC TTT A |
| 118 | Universal-KRAS Reverse Primer 1 | AAG CAT CAT GGC AGT AGT TCT C |
| 119 | KRAS 12 Forward 3 Primer | AGG CCT GCT GAA AAT GAC TG |
| 120 | KRAS 12 Reverse 2 Primer | GCT GTA TCG TCA AGG CAC TC |
| 121 | KRAS-Universal-Q705 Probe | TGA CAC TCA TTC TCT CAA CTC |
| | | TCA TCT |
| 122 | G12C FAM LNA 4 Probe | ACG CCA C+AA GCT CCA |
| 123 | G12VLNA 4 TxRed Probe | TAC GCC A+AC AGC TCC AA |
| 124 | G12S HEX LT | CGC CAC +TAG CTC C |
| 125 | anti-G12A PBNJ 1 | CTA CGC CA+G CAG CTC CAA C |
| 126 | anti-G12D PBNJ | CTACGCCA+TCAGCTCCAAC |
| 127 | anti-G12R PBNJ 1 | ACG CCA C+GA GCT CCA A |
| 128 | 3MOE-2 Anti-Sense | /52MOErA//i2MOErC//i2MOErG/ |
| | | +C+CA C+CA GCT CCA/3SpC3/ |
| 129 | G12A LNA 6 FAM Probe | TAC GCC A+GC AGC TC |
| 130 | G12R LNA 3 TXRED Probe | TAC GCC AC+G AGC TCC |
| 131 | G12D HEX LNA 6 Probe | CGC CA+T CAG CTC CA |
| 132 | anti-G12V PBNJ 3 | CTA CGC CA+A CAG CTC CAA CT |
| 133 | anti-G12C PBNJ 2 | CCT ACG CCA C+AA GCT CCA |
| 134 | G13C LNA 2 HEX Probe | CTA CGC +AAC CAG CTC CA |
| 135 | G13D LNA 2 FAM Probe | CC TAC G+TC ACC AGC TC |

TABLE 2
KRAS exemplary primers and probes

| Oligo | | | | Internal | SEQ ID |
|---|---|---|---|---|---|
| Name | Sequence 5′→3′ | 5′Mod | 3′Mod | Mod | NO: |
| 12fwd3 | AGG CCT GCT GAA AAT | none | none | none | 136 |
| | GAC TG | | | | |
| 12rev2 | GCT GTA TCG TCA AGG | none | none | none | 137 |
| | CAC TC | | | | |
| Uni KRAS | CCC ATG ACA CAA TCC | none | none | none | 138 |
| fwd1 | AGC TTT A | | | | |
| Uni-KRAS- | AAG CAT CAT GGC AGT | none | none | none | 139 |
| rev1 | AGT TCT C | | | | |
| KRAS | TGA CAC TCA TTC TCT | 5Quasar705 | BHQ-2 | Zen | 140 |
| Universal- | CAA CTC TCA TCT | | | | |
| Q705 | | | | | |
| G12C- | ACG CCA C+AA GCT CCA | 56FAM | 3IABkFQ | LNA | 141 |
| FAM-LNA4 | | | | | |
| G12V- | TAC GCC A+AC AGC TCC | 5′ TEX615 | 3IAbRQSp | LNA | 142 |
| LNA4- | AA | | | | |
| TxRed | | | | | |
| G12S HEX | CGC CAC +TAG CTC C | 5HEX | 3IABkFQ | LNA | 143 |
| LT | | | | | |
| G12A- | TAC GCC A+GC AGC TC | 56FAM | 3IABkFQ | LNAs | 144 |
| LNA6-FAM | | | | | |
| G12R- | TAC GCC AC+G AGC TCC | 5TEX615 | 3IAbRQSp | LNA | 145 |
| LNA3- | | | | | |
| TXRED | | | | | |
| G12D- | CGC CA+T CAG CTC CA | 5HEX | 3IABkFQ | LNA | 146 |
| HEX-LNA6 | | | | | |
| G13C- | CTA CGC +AAC CAG CTC | 5HEX | 3IABkFQ | LNA | 147 |
| LNA2-HEX | CA | | | | |
| G13D- | CC TAC G+TC ACC AGC TC | 56FAM | 3IABkFQ | LNA | 148 |
| LNA2-FAM | | | | | |

Table 3 shows exemplary MEOW sequences tested for detection of KRAS mutants, with various 5′, 3′ and internal modifications. Certain sequences included in the Table are preferable, providing good mutation detection. For example, SEQ ID NO: 8, SEQ ID NO: 21, SEQ ID NO: 22, SEQ ID NO: 27, SEQ ID NO: 29, SEQ ID NO: 33, SEQ ID NO: 40, SEQ ID NO: 44, SEQ ID NO: 51, SEQ ID NO: 52, SEQ ID NO: 53 and SEQ ID NO: 63, are exemplary MEOWs that work particularly well. The modifications tested include 2′-Methyl RNA bases to block exonuclease activity; 3′ inverted dT (instead of a 3′ C3 spacer); and oxo-dA. Other alternatives to a C3 spacer include dideoxycytosine and hexanediol. Furthermore, PBNJs may be used to prevent or reduce mutant-on-mutant cross-reactivity. The MEOWs that work less well, are, for example, SEQ ID NO: 10, SEQ ID NO: 11, SEQ ID NO: 13, SEQ ID NO: 14, SEQ ID NO: 14, SEQ ID NO: 15, SEQ ID NO: 16, SEQ ID NO: 17, SEQ ID NO: 17, SEQ ID NO: 18, SEQ ID NO: 19, SEQ ID NO: 20, SEQ ID NO: 24, SEQ ID NO: 25, SEQ ID NO: 26, SEQ ID NO: 28, SEQ ID NO: 34, SEQ ID NO: 35, SEQ ID NO: 36, SEQ ID NO: 37, SEQ ID NO: 39, SEQ ID NO: 59, SEQ ID NO: 60 and SEQ ID NO: 61 including because of inhibition of both wild type and mutant.

TABLE 3
Exemplary MEOW sequences tested for detection of KRAS mutants, with various 5′, 3′
and internal modifications.

| SEQ ID | | | | Internal | |
|---|---|---|---|---|---|
| NO: | Oligo Name | Sequence 5′→3′ | 5′ Mod | Mod | 3′ Mod |
| SEQ ID | PAN-KRAS- | CTA CG+C CA+C | none | LNA | 3SpC3 |
| NO: 1 | PBNJ-2 | +CAG CTC | | | |
| | | CA/3SpC3 | | | |
| | KRAS-PBNJ | TGT GGT AGT | none | LNA | 3SpC3 |
| | Sandwich 1 | TGG AGC | | | |
| | | T+G+G/3SpC3: | | | |
| | | TTG CCT ACG CCA | | | |
| | | +C+C/3SpC3 | | | |
| SEQ ID | | TGT GGT AGT | | | |
| NO: 2 | | TGG AGC | | | |
| | | T+G+G/3 SpC3 | | | |
| SEQ ID | | 3SpC3: TTG CCT | | | |
| NO: 155 | | ACG CCA | | | |
| | | +C+C/3SpC3 | | | |
| | KRAS-PBNJ- | TTG CCT ACG | none | LNA | 3SpC3 |
| | Sandwich 2 | +CCA +C+C | | | |
| | | /3SpC3: GGT AGT | | | |
| | | TGG AGC T+G+G | | | |
| | | TG+G/3SpC3 | | | |
| SEQ ID | | TTG CCT ACG | | | |
| NO: 3 | | +CCA +C+C/3SpC3 | | | |
| SEQ ID | | /3SpC3: GGT AGT | | | |
| NO: 156 | | TGG AGC T+G+G | | | |
| | | TG+G/3SpC3 | | | |
| SEQ ID | S-KRAS- | TTG GAG CT+G | none | LNA | 3SpC3 |
| NO: 4 | PBNJ | +GTG +GCG TAG | | | |
| | | /3SpC3 | | | |
| SEQ ID | S-Pan-KRAS- | TTG GAG CT+G | none | LNAs | 3SpC6 |
| NO: 5 | PBNJ-2 6C | +GTG +GCG TAG | | | |
| | | /3SpC6 | | | |
| SEQ ID | S-Pan-KRAS- | 5SpC3/TTG GAG | 5SpC3 | LNAs | 3SpC3 |
| NO: 6 | PBNJ-2 Dual | CT+G +GTG +GCG | | | |
| | 3Cs | TAG/3SpC3 | | | |
| SEQ ID | S-Pan-KRAS- | 5SpC3/TTG GAG | 5SPC3 | LNAs | 3SpC3 |
| NO: 7 | PBNJ-2 3C- | CT+G +GTG +GCG | | | |
| | 6C | TAG/3SpC3 | | | |
| SEQ ID | S-PAN- | /52MOErA/GT TGG | /52MOErA/ | LNA | 3InvdT |
| NO: 8 | KRAS-PBNJ | AGC T+G+G TG+G | | | |
| | 3 InvdT | CGT/3InvdT | | | |
| SEQ ID | S-PAN- | /5dSp/TGG AGC | /5dSp/ | LNA | 3C6 |
| NO: 9 | KRAS-PBNJ | T+G+G TG+G CGT | | | |
| | 4 abasic | AGG C/3C6 | | | |
| SEQ ID | S-PAN- | T/idSp/G GAG | none | idSp, | /3SpC3/ |
| NO: 10 | KRAS-PBNJ | CT+G +GTG +GCG | | LNAs | |
| | 4 abasic-2 | TAG GC /3SpC3/ | | | |
| SEQ ID | S-PAN- | T*G*G* A*G*C | none | PS | /3SpC3/ |
| NO: 11 | KRAS-PBNJ | T+G+G TG+G CGT* | | bonds, | |
| | PS/3C | A*G*G* C/3SpC3/ | | LNAs | |
| SEQ ID | S-PAN- | /52MOErT/*G*G* | MOE T | PS | 3SpC3 |
| NO: 12 | KRAS-PBNJ | A*G*C T+G+G | | bonds, | |
| | 5MOE/PS/3C | TG+G CGT* | | LNAs | |
| | | A*G*G* C/3SpC3/ | | | |
| SEQ ID | S-PBNJ Bulk | +G+G+T +G+GC | none | LNA | 3SpC3 |
| NO: 13 | LNA | GTA GGC | | | |
| | | AA/3SpC3/ | | | |
| SEQ ID | S-PBNJ | A*G*T* T*G*G | none | LNA, PS | 3SpC3 |
| NO: 14 | PS/LNA-1 | AGC T+G+G TG+G | | bonds | |
| | | CGT AG*G* | | | |
| | | C*A*A* G/3SpC3/ | | | |
| SEQ ID | S-PBNJ | A*G*T* T*G*G | none | LNA, PS | 3SpC3 |
| NO: 15 | PS/LNA-2 | AGC T+G+G TG+G | | bonds | |
| | | CGT AGG CAA | | | |
| | | G/3SpC3/ | | | |
| SEQ ID | S-PBNJ | A*G*T* T*GG AGC | none | LNA, PS | 3SpC3 |
| NO: 16 | PS/LNA-3 | T+G+G TG+G CGT | | bonds | |
| | | AGG CAA G/3SpC3/ | | | |
| SEQ ID | S-PBNJ | A*G*T* T*GG AGC | none | LNA, PS | 3SpC3 |
| NO: 17 | PS/LNA-4 | T+G+G TG+G CGT | | bonds | |
| | | AGG* C*A*A* | | | |
| | | G/3SpC3/ | | | |
| SEQ ID | S-PBNJ | A*G*C* T*+G+GG | none | LNA, PS | 3SpC3 |
| NO: 18 | PS/LNA-5 | TG+G CGT* | | bonds | |
| | | A*G*G* C/3SpC3/ | | | |
| SEQ ID | S-PBNJ | A*G*C* T*+G+G | none | LNA, PS | 3SpC3 |
| NO: 19 | PS/LNA-6 | TG+G CGT AGG | | bonds | |
| | | C/3SpC3/ | | | |
| SEQ ID | S-PBNJ | /52MOErG//i2MOErC// | /52MOErG/ | /i2MOErC// | 3SpC3 |
| NO: 20 | 3MOE | i2MOErT/+G+GT | | i2MOErT/, | |
| | | G+GCGTAGGC | | LNAs | |
| | | /3SpC3/ | | | |
| SEQ ID | S-PBNJ | /52MOErG/*/i2MOErC/ | /52MOErG/ | /i2MOErC// | 3′ Mod |
| NO: 21 | 3MOE/PS-1 | */i2MOErT/*+G+ | | i2MOErT/, | |
| | | GTG+GCGTAGGC | | LNAs, | |
| | | /3SpC3/ | | PS | |
| SEQ ID | S-PBNJ | /52MOErG/*/i2MOErC/ | /52MOErG/ | /i2MOErC// | 3SpC3 |
| NO: 22 | 3MOE/PS-2 | */i2MOErT/*+G+ | | i2MOErT/, | |
| | | GTG+GCGTAGGC | | LNAs, | |
| | | /3SpC3/ | | PS | |
| SEQ ID | S-PBNJ Bulk | +T+G+GTG +GC | none | LNA | 3SpC3 |
| NO: 23 | LNA-2 | GTA GGC | | | |
| | | AA/3SpC3/ | | | |
| SEQ ID | S-PBNJ Bulk | +T+G+GAGCT+G+ | none | LNA | 3SpC3 |
| NO: 24 | LNA-3 | GTG+GCGTAGGC/ | | | |
| | | 3SpC3/ | | | |
| SEQ ID | S-PBNJ- Bulk | +G+G+T +GGC | none | LNA | 3SpC3 |
| NO: 25 | LNA5 | GTA GGC | | | |
| | | AA/3SpC3/ | | | |
| SEQ ID | S-PBNJ 5 | /5dSp/AGC T+G+G | /5dSP/ | LNA | 3SpC6 |
| NO: 26 | abasic-3 | T+G+G CGT AGG | | | |
| | | CAAG/3SpC3/ | | | |
| SEQ ID | S-PBNJ 6 | /5dSp/AGCT+G+GT | /5dSP/ | LNA | 3SpC3 |
| NO: 27 | Abasic 4 | GGCGTAGGCAAG | | | |
| | | A/3SpC3/ | | | |
| SEQ ID | S-PBNJ 6 | /5dSp/AGCT+G+GT | /5dSP/ | LNA | 3SpC3 |
| NO: 28 | Abasic 5 | +GGCGTAGGCAA | | | |
| | | GA/3SpC3/ | | | |
| SEQ ID | S-PBNJ- | TGG[OXOA]GCTG | none | 8-oxoA | 3InvdT |
| NO: 29 | oxodA | GTGGCGTAGGC/ | | | |
| | | 3SpC3 | | | |
| SEQ ID | S-PAN- | /5dSp/TGG AGC | /5dSp/ | LNA | 3C6 |
| NO: 30 | KRAS-PBNJ | T+G+G TG+G CGT | | | |
| | Abasic-3C | AGG C | | | |
| SEQ ID | S-PBNJ Bulk | /+G+G+T +G+GC | none | LNA | /3SpC3/ |
| NO: 31 | LNA-3C | GTA GGC AA | | | |
| SEQ ID | S-PBNJ 5 | /5dSp/AGC T+G+G | /5dSP/ | LNA | /3SpC3/ |
| NO: 32 | abasic-3 | T+G+G CGT AGG | | | |
| | | CAAG/3SpC3/ | | | |
| SEQ ID | S-PBNJ Bulk | +G+G+C+G+TAGG | none | LNA | 3SpC3 |
| NO: 33 | LNA-4 | CAAGA/3SpC3/ | | | |
| SEQ ID | S-PBNJ 6 | /5dSp/CT+G+GT+G | /5dSp/ | LNA | 3SpC3 |
| NO: 34 | Abasic 1/2 | +GCGTAGGCAAG | | | |
| | | A/3SpC3/ | | | |
| SEQ ID | S-PBNJ 6 | /5dSp/CG+C +CA+C | /5dSp/ | LNA | 3SpC3 |
| NO: 35 | Abasic 2/2 | +CAG CTC CAA | | | |
| | | CTA/3SpC3/ | | | |
| SEQ ID | S-PBNJ Bulk | +G+G+T +G+GC | none | LNA | 3SpC3 |
| NO: 36 | LNA 1/2 | GTA GGC AAG | | | |
| | | /3SpC3/ | | | |
| SEQ ID | S-PBNJ Bulk | +C+C+A +C+CA | none | LNA | 3SpC3 |
| NO: 37 | LNA 2/2 | GCT CCA ACT | | | |
| | | ACC/3SpC3/ | | | |
| SEQ ID | S-PBNJ 6 | /5dSp/AGCT+G+GT | /5dSP/ | LNA | 3SpC3 |
| NO: 38 | Abasic 4 | GGCGTAGGCAAG | | | |
| | | A/3SpC3/ | | | |
| SEQ ID | S-PBNJ 6 | /5dSp/AGCT+G+GT | /5dSP/ | LNA | 3SpC3 |
| NO: 39 | Abasic 5 | +GGCGTAGGCAA | | | |
| | | GA/3SpC3/ | | | |
| SEQ ID | S-PBNJ- Bulk | /+G+G+T +GGC | none | LNA | 3SpC3 |
| NO: 40 | LNA5 | GTA GGC | | | |
| | | AA/3SpC3/ | | | |
| SEQ ID | Anti-S-PBNJ- | +G+C+C AC+C | none | LNA | 3SpC3 |
| NO: 41 | Bulk LNA 2 | AGC TCC AAC | | | |
| | | /3SpC3/ | | | |
| SEQ ID | Anti-S-PBNJ- | /5dSp/CCT ACG | /5dSp/ | LNA | 3′ Mod |
| NO: 42 | Abasic 1 | +CCA +C+CA GCT | | | |
| | | CCA A/3SpC3/ | | | |
| SEQ ID | Anti-S-PBNJ | /52MOErA//i2MOErC// | /52MOErA/ | /i2MOErC// | 3SpC3 |
| NO: 43 | 3MOE | i2MOErG/+CCA | | i2MOErG/, | |
| | | +C+CA GCT CCA | | LNAs | |
| | | /3SpC3/ | | | |
| SEQ ID | Anti-S-PBNJ | /52MOErA//i2MOErC// | /52MOErA/ | /i2MOErC// | 3SpC3 |
| NO: 44 | 3MOE-2 | i2MOErG/ | | i2MOErG/, | |
| | | +C+CA C+CA GCT | | LNAs | |
| | | CCA/3SpC3/ | | | |
| SEQ ID | Pseudo- | /5dSp/GCT CCC | /5dSP/ | LNA | 3SpC3 |
| NO: 45 | Abasic | AG+A CCT GCT | | | |
| | Blocker 1 | GAA A/3SpC3/ | | | |
| SEQ ID | Pseudo- | /52MOErG//i2MOErC// | /52MOErA/ | /i2MOErC// | 3SpC3 |
| NO: 46 | 3MOE- | i2MOErT/CCC | | i2MOErG/, | |
| | Blocker 1 | AG+A CCT GCT | | LNA | |
| | | GAA A/3SpC3/ | | | |
| SEQ ID | Pseudo- | /5dSp/AGC GGC | /5dSP/ | LNA | 3SpC6 |
| NO: 47 | Abasic | TCC CAG +ACC | | | |
| | Blocker 2 | /3SpC3/ | | | |
| SEQ ID | Pseudo- | /5dSp/AG+A CCT | /5dSP/ | LNA | 3SpC3 |
| NO: 48 | Abasic | GCT GAA AAT | | | |
| | Blocker 3 | GAC TG/3SpC3/ | | | |
| SEQ ID | Pseudo-Rev- | /5dSp/GGA TCA | /5dSP/ | LNA | 3SpC3 |
| NO: 49 | Abasic-1 | TAT T+GG TCC | | | |
| | | ACA AA/3SpC3/ | | | |
| SEQ ID | Pseudo-Rev- | /5dSp/CTC TAT | /5dSP/ | LNA | 3InvdT |
| NO: 50 | Abasic-2 | TGT TGG ATC ATA | | | |
| | | TT+G GTC | | | |
| | | C/3SpC3/ | | | |
| SEQ ID | AS- | /52MOErT//i2MOErC// | /52MOErT/ | /i2MOErC// | 3C6 |
| NO: 51 | 3MOE + Bulk | i2MOErT/TGC | | i2MOErT/, | |
| | LNA Blocker | CTA CG+C +CA+C | | LNA | |
| | 1 | +C/3SpC3/ | | | |
| SEQ ID | AS- | /5dSp/TCT TGC | /5dSP/ | LNA | /3SpC3/ |
| NO: 52 | Abasic + Bulk | CTA CG+C +CA+C | | | |
| | LNA Blocker | +C/3SpC3/ | | | |
| | 1 | | | | |
| SEQ ID | Sense-Bulk | /+G+G+T +GGC | none | LNA | /3SpC3/ |
| NO: 53 | LNA 6 hi TM | GTA GGC AAG | | | |
| | | AGT GC | | | |
| SEQ ID | Anti-S | /52MOErC//i2MOErC// | /52MOErC/ | /i2MOErC// | 3SpC3 |
| NO: 54 | 3MOE-3 | i2MOErT/ACG | | i2MOErT/, | |
| | | +C+CA C+CA GCT | | LNAs | |
| | | CC/3SpC3/ | | | |
| SEQ ID | Sense-Bulk | +G+GT +G+GC | none | LNA | 3SpC3 |
| NO: 55 | LNA7 | GTA GGC | | | |
| | | AA/3SpC3/ | | | |
| SEQ ID | Sense-TOM | mGAG CT+G +GTG | 2′-O- | 2′-O- | 3SpC3 |
| NO: 56 | blocker-1 | +GCG TAG/3SpC3/ | Methyl | Methyl | |
| SEQ ID | Sense-TOM | mGmAG CT+G | 2′-O- | 2′-O- | 3SpC3 |
| NO: 57 | blocker-2 | +GTG +GCG TAG | Methyl | Methyl | |
| | | /3SpC3/ | | | |
| SEQ ID | Sense-TOM | mGmAmG CT+G | 2′-O- | 2′-O- | 3 SpC3 |
| NO: 58 | blocker-3 | +GTG +GCG | Methyl | Methyl | |
| | | TA/3SpC3/ | | | |
| SEQ ID | Sense-TOM | mGmAmG mCT+G | 2′-O- | 2′-O- | 3SpC3 |
| NO: 59 | blocker-4 | +GTG +GCG | Methyl | Methyl | |
| | | TA/3SpC3/ | | | |
| SEQ ID | Sense-TOM | mGmGmA mGmCT | 2′-O- | 2′-O- | 3SpC3 |
| NO: 60 | blocker-5 | +G+GT G+GC GTA | Methyl | Methyl | |
| | | /3SpC3/ | | | |
| SEQ ID | Sense-TOM | mGmGmA | 2′-O- | 2′-O- | 3SpC3 |
| NO: 61 | blocker-6 | mGmCmU +G+GT | Methyl | Methyl | |
| | | G+GC GTA /3SpC3/ | | | |
| SEQ ID | Sense-TOM | mGmGmU mGGC | 2′-O- | 2′-O- | 3SpC3 |
| NO: 62 | blocker-7 | GTA GGC AAG | Methyl | Methyl | |
| | | AGT GC/3SpC3/ | | | |
| SEQ ID | Anti-S-TOM | mUmAmC mG+CC | 2′-O- | 2′-O- | /3SpC3/ |
| NO: 63 | blocker 1 | A+C+C AGC | Methyl | Methyl | |
| | | TC/3SpC3/ | | | |

Together, the sequences of TABLES 1-4 facilitate multiplex detection of KRAS gene mutations G12C, G12V, G12S, G12A, G12R, G12D, G13C, and G13D. The kit may further comprise a variety of positive controls, including for WT, G12C, G12V, G12S, G12A, G12R, G12D, G13C and G13D positive controls. Accordingly, the methods and kits described herein may use one or more of the described sequences, including any combinations thereof.

TABLE 4
Exemplary MEOW sequences.

| SEQ ID | | | | Internal | |
|---|---|---|---|---|---|
| NO: | Oligo Name | Oligo Sequence | 5 Mod | Mod | 3′ Mod |
| SEQ ID | BCR Ex1 LNA | +A+A+G +A+CC | LNA | LNAs | None |
| NO: 64 | Forward Primer | GGG CAG ATC | | | |
| SEQ ID | BCR Ex1 ROX | /56-ROXN/CG CCT | 56- | None | 3IAbRQSp |
| NO: 65 | Probe | TCC ATG GAG ACG | ROXN | | |
| | | CA/3IAbRQSp/ | | | |
| SEQ ID | BCR Ex6 LNA | +A+C+C +T+GA | LNA | LNAs | None |
| NO: 66 | Forward Primer | GAG CCA GAA | | | |
| SEQ ID | BCR Ex6 Cy5.5 | /5Cy55/CCA AGG | 5Cy55 | LNAs | 3IAbRQSp |
| NO: 67 | Probe | A+TC +CAA CGA | | | |
| | | CCA AG/3IAbRQSp/ | | | |
| SEQ ID | BCR Ex13 | +T+G+A +C+CA | LNA | LNAs | None |
| NO: 68 | LNA v3.0 | ACT CGT GTG | | | |
| | Forward Primer | | | | |
| SEQ ID | BCR Ex13 | /56-FAM/CA GAC | 56-FAM | LNAs | 3IABkFQ |
| NO: 69 | FAM Probe | TGT CCA +CAG | | | |
| | | CAT TCC | | | |
| | | G/3IABkFQ/ | | | |
| SEQ ID | BCR Ex19 | +C+A+A +G+AG | LNA | LNAs | None |
| NO: 70 | LNA v2.0 | AGA GAG GTC CAA | | | |
| | Forward Primer | G | | | |
| SEQ ID | BCR Ex19 Cy5 | /5Cy5/CT +ACA TCG | 5Cy5 | LNAs | 3IAbRQSp |
| NO: 71 | Probe | TGC GC+C AGT | | | |
| | | G/3IAbRQSp/ | | | |
| SEQ ID | ABL Ex3 v2.0 | GCC TAA GAC CCG | None | None | None |
| NO: 72 | Reverse Primer | GAG CTT TTC AC | | | |
| SEQ ID | ABL Ex6 v2.0 | T+A+T +CAT CAC | None | LNAs | None |
| NO: 73 | Forward Primer | TGA GTT CAT GAC | | | |
| | | CTA CG | | | |
| SEQ ID | ABL Ex6 HEX | /5HEX/CG TGG | 5HEX | LNAs | 3IABkFQ |
| NO: 74 | Probe | TG +C TG +C TGT | | | |
| | | ACA TG/3IABkFQ/ | | | |
| SEQ ID | ABL Uni v2.0 | CTGTCATCAACCT | None | None | None |
| NO: 75 | Reverse Primer | GCTCAGGC | | | |
| SEQ ID | ABL Ex3 MOE | /52MOErC//i2MOErC | 52MOErC | i2MOErC, | 3SpC3 |
| NO: 76 | MEOW | //i2MOErT/TCG TAT | | i2MOErT | |
| | | CTC AGC GAG ATG | | | |
| | | GAC C/3SpC3/ | | | |
| SEQ ID | BCR Ex4 MOE | /52MOErT//i2MOErC | 52MOErT | i2MOErC, | 3SpC3 |
| NO: 77 | MEOW | //i2MOErC/TTG | | i2MOErC | |
| | | TGG ATC TCG TAG | | | |
| | | AGC TCA G/3SpC3/ | | | |
| SEQ ID | BCR Ex10 | /52MOErC//i2MOErT | 52MOErC | i2MOErT, | 3SpC3 |
| NO: 78 | MOE MEOW | //i2MOErC/CAG | | i2MOErC | |
| | | TTC ATC CAC CAT | | | |
| | | CTG GAA | | | |
| | | GC/3SpC3/ | | | |
| SEQ ID | BCR Ex16 | /52MOErG//i2MOErG | 52MOErG | i2MOErG, | 3SpC3 |
| NO: 79 | MOE MEOW | //i2MOErG/+ATC | | i2MOErG, | |
| | | TTC GTC TTG TTG | | LNAs | |
| | | TAA CAC/3SpC3/ | | | |
| SEQ ID | TYM_tef1_BF2_ | RGCYGGTATYTCY | None | None | None |
| NO: 80 | FWD | AAGGAYGG | | | |
| SEQ ID | TYM_tef1_BF2_ | GTGRTGCATYTCR | None | None | None |
| NO: 81 | Rev1 | ACRGAYTT | | | |
| SEQ ID | TYM_tef1_BF2_ | GTGGTGCATTTCA | None | None | None |
| NO: 82 | Rev2 | ACACTCTT | | | |
| SEQ ID | TYM_tef1_BF2_ | /56-FAM/TG GM+A | 56-FAM | LNAs | 3IABkFQ |
| NO: 83 | NP1_FAM | YG+G TGA CAA | | | |
| | | C+A+T G/3IABkFQ/ | | | |
| SEQ ID | TYM_tef1_BF2_ | /56-FAM/TT CAA | 56-FAM | LNAs | 3IABkFQ |
| NO: 84 | NP2_FAM | C+G+G YGA C+AA | | | |
| | | C+AT G/3IABkFQ/ | | | |
| SEQ ID | TYM_tef1_BF2_ | /56-FAM/TK SM+A | 56-FAM | LNAs | 3IABkFQ |
| NO: 85 | NP3_FAM | CG+G TGA YAA | | | |
| | | CA+T G/3IABkFQ/ | | | |
| SEQ ID | TYM_tef1_BF2_ | /56-FAM/TT CMA | 56-FAM | LNAs | 3IABkFQ |
| NO: 86 | NP4_FAM | CG+G H+G+A CAA | | | |
| | | YAT G/3IABkFQ/ | | | |
| SEQ ID | TYM_tef1_BF2_ | /56-FAM/TG GCA | 56-FAM | LNAs | 3IABkFQ |
| NO: 87 | NP5_FAM | CGG +AGA CAA | | | |
| | | GTG A/3IABkFQ/ | | | |
| SEQ ID | Ext-2-FWD | AYG AAC AAA | None | None | None |
| NO: 88 | | GCA TCC CAC CT | | | |
| SEQ ID | Ext-2-REV | TGG AGC ATA CAC | None | None | None |
| NO: 89 | | AGT ACA YVCR | | | |
| | Ext-2-HEX | /5HEX/CTT CCA | 5HEX | ZEN | 3IABkFQ |
| | | CSG/ZEN/CAT CRT | | | |
| | | CAT WAA TGA | | | |
| | | TCT/3IABkFQ | | | |
| SEQ ID | | ZEN/CAT CRT CAT | | | |
| NO: 90 | | WAA TGA | | | |
| | | TCT/3IABkFQ | | | |
| SEQ ID | TYM_tef1_BF2_ | TTC +GA+G GGT | None | LNAs | 3SpC3 |
| NO: 91 | Cann_PBNJ | GAC AAC ATG | | | |
| | | /3SpC3/ | | | |
| SEQ ID | TYM_tef1_BF2_ | +G+A+T +T+TG | LNA | LNAs | 3SpC3 |
| NO: 92 | cann_bulkLNA3_ | CTG TTG TAA CAA | | | |
| | mod | GGT/3SpC3/ | | | |
| SEQ ID | E380_Fwd2 | TGTGGATTTGACC | None | None | None |
| NO: 93 | | CTCCATGA | | | |
| SEQ ID | E380_Rev2 | CCAATCATCAGGA | None | None | None |
| NO: 94 | | TCTCTAGCCA | | | |
| SEQ ID | E380Q_Probe2_ | /56- | 56- | LNAs | 3IAbRQSp |
| NO: 95 | ROX | ROXN/TCCACCTTC | ROXN | | |
| | | TA+CAATGTGCCT/ | | | |
| | | 3IAbRQSp/ | | | |
| SEQ ID | S463_Fwd | TCT CTG CGC ATT | None | None | None |
| NO: 96 | | CAG GAG TG | | | |
| SEQ ID | S463_Rev | GTG GAT ATG GTC | None | None | None |
| NO: 97 | | CTT CTC TTC CAG | | | |
| SEQ ID | S463P_Probe_ | /5Cy55/CAC ATT | 5Cy55 | LNA | 3IAbRQSp |
| NO: 98 | Cy5.5 | TCT G+CC CAG | | | |
| | | CAC C/3IAbRQSp/ | | | |
| SEQ ID | ESR L536 | GGC ATG GAG CAT | None | None | None |
| NO: 99 | Fwd2 | CTG TAC AGC | | | |
| SEQ ID | D538_Rev | TGGGCGTCCAGCA | None | None | None |
| NO: 100 | | TCTC | | | |
| SEQ ID | L536R_Probe | TGGTGCCCC+GCT | 5Cy5 | LNAs | 3IAbRQSp |
| NO: 101 | Cy5 | ATGAC | | | |
| SEQ ID | L536H_Probe | TGGTGCCCC+ACT | 5Cy5 | LNAs | 3IAbRQSp |
| NO: 102 | Cy5 | ATGACC | | | |
| SEQ ID | L536P_Probe | TGGTGCCCC+CCT | 5Cy5 | LNAs | 3IAbRQSp |
| NO: 103 | Cy5 | ATGAC | | | |
| SEQ ID | Y537C_Probe | TGCCCCTCT+GTG | 5HEX | LNA | 3IABkFQ |
| NO: 104 | HEX | ACCTG | | | |
| SEQ ID | Y537S_Probe | TGGTGCCCCTCT+C | 5HEX | LNA | 3IABkFQ |
| NO: 105 | HEX | TGACCT | | | |
| SEQ ID | Y537N_Probe | TGCCCCTC+AATG | 5HEX | LNA | 3IABkFQ |
| NO: 106 | HEX | ACCTGC | | | |
| SEQ ID | D538G_Probe | CCTCTATG+GCCT | 56-FAM | LNA | 3IABkFQ |
| NO: 107 | FAM | GCTGCT | | | |
| SEQ ID | E380_PBNJ2 | TCCACCTTCTA+G | None | LNA | 3 SpC3 |
| NO: 108 | | AATGTGCCT/3SpC3/ | | | |
| SEQ ID | S463_PBNJ-2 | TA CAC ATT TCT | None | LNA | 3 SpC3 |
| NO: 109 | | G+TC CAG CAC | | | |
| | | CCT/3SpC3/ | | | |
| SEQ ID | ESR1 536-538 | /52MOErC//i2MOErC | 52MOErC | i2MOErC, | 3SpC3 |
| NO: 110 | 3MOE Blocker | //i2MOErC/+TC | | LNAs | |
| | | +T+AT G+AC CTG C | | | |
| | | TGC T/3SpC3/ | | | |
| SEQ ID | 536-538 ESR1 | CCC +TC+T +ATG | None | LNA | 3 SpC3 |
| NO: 111 | WT PBNJ-1 | +ACC TGC TGC | | | |
| | | T/3SpC3/ | | | |
| SEQ ID | 536-538 ESR1 | GCC CCT CTA TGA | None | None | 3 SpC3 |
| NO: 112 | WT PBNJ-2 | CCT GCT GCT | | | |
| | | /3SpC3/ | | | |
| SEQ ID | NPM1-SK- | GAT GTT GAA CTA | None | LNAs | None |
| NO: 113 | fwd2- | TG+C AAA G+AG | | | |
| | | AC | | | |
| SEQ ID | RP3 | ggacagccagatatcaactgtt | None | None | None |
| NO: 114 | | aca | | | |
| SEQ ID | NPM1-SK-uni- | TGT GGT TAA GGA | 56-FAM | None | 3IABkFQ |
| NO: 115 | FAM2- | ACC ACA ACA | | | |
| SEQ ID | NPM1- | CCA CT+G +C+C+A | None | LNAs | 3 SpC3 |
| NO: 116 | bulkLNA-SK8 | GAG ATC TTG AAT | | | |
| | | AGC CTG G/3SpC3/ | | | |

Table 4 shows exemplary MEOW sequences used throughout herein. SEQ ID NOs: 64-79 were used to obtain the results from a multiplexed assay targeting multiple BCR-ABL1 fusion types tested against RNA containing the e19a2 fusion type (exon 19 of BCR fused to exon 2 of ABL) depicted undesired amplification coming from an upstream primer and FAM probe on exon 13 of BCR as shown in FIG. 14. SEQ ID NOs: 80-92 were used to obtain the results for multiplexed assay targeting total yeast and mold (TYM) for routine cannabis testing demonstrated optimal specificity with the use of a GT-blocker in combination with a C3 PBNJ depicted in FIG. 17. SEQ ID NOs: 93-112 were used to obtain the results from a multiplexed typing assay targeting common SNVs in ESR1 (i.e., D538G (FIG. 12, top) and Y537C/S/N as depicted in FIG. 12 and FIG. 13. SEQ ID NOs: 113-116 were used to obtain the results depicted in FIG. 7B.

Table 5 shows additional designs for MEOWs (GT-Blockers) including, for example, 2′ Amino, 2′ Fluro, or hairpin modifications.

TABLE 5
Exemplary MEOW sequences.

| SEQ ID | | | Internal | |
|---|---|---|---|---|
| NO: | Oligo Sequence | 5′ Mod | Mod | 3′ Mod |
| 149 | [2-AmC][2-AmC]T | 2-AmC | LNA | 3SpC3 |
| | ACG+C+CAC+CAGCTCC/3SpC3/ | | | |
| 150 | [fC]*[fC]*[fT]* | [fC] | PS bond, | 3SpC3 |
| | ACG+C+CAC+CAGTCC/SpC3/ | | LNA | |
| 151 | GCG AAG CTA CG+C +CAC | none | LNA | 3SpC3 |
| | +CAG CT/SpC3/ | | | |

Table 6 shows additional designs for anti-blocker sequences, said sequences are examples of suitable anti-blocker primers and do not contain any labels or modifications.

TABLE 6
Exemplary Anti-Blocker sequences.

| SEQ ID NO: | Oligo Name | Oligo Sequence |
|---|---|---|
| 152 | G13D_Fwd Primer 1 | TGG TAG TTG GAG CTG GTG A |
| 153 | G12D-Fwd Primer | ACT TGT GGT AGT TGG AGC TGA |
| 154 | G12C_Fwd Primer 1 | GAA TAT AAA CTT GTG GTA GTT |
| | | GGA GCT T |

### STATEMENTS REGARDING INCORPORATION BY REFERENCE AND VARIATIONS

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of screening for a target sequence from a reference sequence in a biological sample by polymerase chain reaction (PCR), the method comprising the steps of:
providing a labelled probe comprising a fluorophore and a quencher, wherein the labelled probe has a shared sequence region configured to hybridize to a shared region of the target sequence and the reference sequence;
providing a mutant enhancing oligonucleotide wall (MEOW) comprising:
a 5′ exonuclease resister;
a 3′ extension blocker configured to prevent elongation by a polymerase;
a reference binding region positioned between the 5′ exonuclease resister and the 3′ extension blocker, wherein the reference binding region is configured to hybridize to a binding reference region of the reference sequence at a higher binding affinity than a corresponding binding target region of the target sequence;
wherein the shared region of the target sequence and reference sequence is positioned downstream from:
the binding reference region of the reference sequence; and
the binding target region of the target sequence;
performing a PCR on a PCR solution comprising:
the biological sample containing the reference and/or target sequence;
the labelled probe;
the MEOW;
PCR reagents;
optically detecting an amplicon from the performing the PCR step; and
identifying the biological sample as containing the target sequence for the optically detected amplicon;
thereby screening for the target sequence from the reference sequence.

2. A method of typing a target sequence from a reference sequence in a biological sample by polymerase chain reaction (PCR), the method comprising the steps of:
providing a labelled probe comprising a fluorophore and a quencher, wherein the labelled probe has a labelled probe sequence region configured to hybridize to a target sequence region of the target sequence;
providing a MEOW comprising:
a 5′ exonuclease resister;
a 3′ extension blocker configured to prevent elongation by a polymerase;
a reference binding region positioned between the 5′ exonuclease resister and the 3′ extension blocker, wherein the reference binding region is configured to hybridize to a binding reference region of the reference sequence;
wherein:
the labelled probe hybridizes to the target sequence region at a higher binding affinity than the MEOW non-specific binding to the target sequence region; and
the MEOW hybridizes to the binding reference region at a higher binding affinity than the labelled probe non-specific binding to the binding reference region;
performing a PCR on a PCR solution comprising:
the biological sample containing the reference and/or target sequence;
the labelled probe;
the MEOW;
forward and reverse primers;
PCR reagents;
optically detecting an amplicon from the performing the PCR step; and
typing the biological sample as containing the target sequence for the optically detected amplicon;
thereby typing the target sequence from the biological sample.

3. The method of claim 1, wherein the MEOW 5′ exonuclease resister is selected from the group consisting of an Abasic site, consecutive locked nucleic acids (LNAs), consecutive phosphorothioate (PS) bonds, consecutive 2′-O-methoxyethyl (MOE) bases, consecutive 2′-O-Methyl (2′OMe), a MOE and a PS combination, and any combination thereof.

4. The method of claim 1, wherein the MEOW:
inhibits nonspecific SNV probe hybridization of a reference sequence and blocks reference sequence amplification to detect a short nucleotide variant (SNV), insertions, deletions, and/or fusions; or
substantially inhibits polymerase synthesis and amplification of a wild-type sequence to provide a screening assay for various mutations located in close proximity.

5. The method of claim 1, wherein the biological sample is selected from the group consisting of:
viruses, wherein the reference sequence is from a wild-type virus or a parent virus and the target sequence comprises at least one mutation compared to the reference sequence;
mammalian cells, wherein the reference sequence is reflective of a low-disease condition state and the target sequence has one or more nucleotide changes compared to the reference sequence reflective of an elevated disease condition risk or the presence of disease;
circulating cell free or tumor DNA, wherein the reference sequence is somatic, wild-type sequence and the target sequence originated in a tumor or cancerous cell and has one or more nucleotide changes compared to the reference sequence reflective of an elevated disease condition risk or the presence of disease;
circulating cell free fetal DNA, wherein the reference sequence is reflective of the maternal DNA sequence and the target sequence has one or more nucleotide changes compared to the reference sequence reflective of the fetus DNA sequence;
bacteria, wherein the reference sequence is from a wild-type bacterium or one species of bacteria and the target sequence comprises at least one variation compared to the reference sequence;
fungus, wherein the reference sequence is from a wild-type fungus or one species of fungus and the target sequence comprises at least one variation compared to the reference sequence; and
plants, wherein the reference sequence is from a wild-type plant or one species of plant and the target sequence comprises at least one variation compared to the reference sequence.

6. (canceled)

7. The method of claim 1, wherein the reference and target sequences differ by:
a single nucleotide substitution;
a nucleotide insertion of one or more nucleotides;
a nucleotide deletion of one or more nucleotides; and/or
a fusion construct.

8. (canceled)

9. The method of claim 1, wherein the PCR is selected from the group consisting of ddPCR, dPCR, qPCR, RT-ddPCR, RT-dPCR, and RT-qPCR.

10. (canceled)

11. (canceled)

12. (canceled)

13. The method of claim 1, for detection of a SNV, insertion, deletion, or fusion containing DNA or RNA sequences.

14. The method of claim 1, wherein the PCR is dPCR and the dPCR comprises partition or droplet-based PCR and the MEOW reduces or eliminates signal associated with a lower efficiency, non-specific off-target amplification, thereby increasing a signal to noise ratio for specific amplification of the target sequence.

15. The method of claim 1, further comprising the steps of:
tuning a probe output amplitude by providing the MEOW at a lower concentration, that the concentration of the labelled probe and detecting a plurality of probe output amplitudes for multiplex detection of a plurality of target sequences in a single or a multichannel fluorescence detector.

16. The method of claim 1, wherein the target and reference sequence differ by a single nucleotide mismatch that is a single nucleotide variant or is part of a short nucleotide variant, an insertion, a deletion, or a fusion event.

17. (canceled)

18. (canceled)

19. (canceled)

20. The method of claim 1, wherein the extension blocker is a 3′ carbon-based spacer such as C3, C6, or C12, inverted dT/ddT, or a 3′ quencher.

21. The method of claim 1, wherein the MEOW contains a locked nucleic acid (LNA) at a SNV position.

22. (canceled)

23. The method of claim 1, further comprising providing a plurality of labelled probes, a plurality of MEOWs, or a plurality of labelled probes and a plurality of MEOWs, including a plurality of MEOWs that hybridize to every possible SNV at the binding reference region.

24. (canceled)

25. The method of claim 1, used in a biological sample to test for mutations associated with an elevated risk or presence of a disease condition associated with a predictive nucleotide sequence, including a cancer, a neurodegenerative condition, or a reproductive condition.

26. The method of claim 1, used in a biological sample to test for a variant of a pathogen, including a pathogen that is a virus, a bacteria, or a fungus.

27. The method of claim 1, wherein the biological sample is from wastewater, environmental sample, bodily fluid, tissue, cell culture, plant, or tumor.

28. (canceled)

29. (canceled)

30. The method of claim 1, wherein a ratio of MEOW concentration to labelled probe concentration is: equimolar or greater; or less than equimolar.

31. The method of claim 1, wherein the reference binding region corresponds to a region of hypermutability of a reference sequence, including a KRAS G12/G13 sequence.

32. The method of claim 1, having a limit of detection for a mutation compared to wild-type or parent down to a 0.1% variant allele frequency.

33. The method of claim 1, further comprising the step of providing an anti-blocker to promote amplification of a proto-oncogene by decreasing MEOW hybridization to the binding reference region and wherein the anti-blocker is a primer sequence that hybridizes to a SNP site.

34. (canceled)

35. The method of claim 1, wherein the MEOW substantially inhibits wild type amplicon synthesis.

36. A kit for screening of a target sequence from a reference sequence in a biological sample by polymerase chain reaction (PCR), the kit comprising:
at least one forward and reverse primer pair for amplifying both a reference strand having a reference sequence and a target strand having a target sequence;
a labelled probe comprising a fluorophore and a quencher;
a mutant enhancing oligonucleotide wall (MEOW);
wherein the labeled probe hybridizes to the target sequence region at a higher binding affinity than the MEOW non-specifically binds to the target sequence region and the MEOW hybridizes to the binding reference region at a higher binding affinity than the labelled probe non-specific binding to the binding reference region;
optionally:
a positive control for the reference sequence;
a positive control for the target sequence; and
a mixed control comprising the reference sequence and the target sequence.

37. A kit for typing a target sequence from a reference sequence, the kit comprising:
at least one forward and reverse primer pair for amplifying both a reference strand having a reference sequence and a target strand having a target sequence;
a labelled probe comprising a fluorophore and a quencher;
a mutant enhancing oligonucleotide wall (MEOW);
wherein the labeled probe hybridizes to the target sequence region at a higher binding affinity than the MEOW non-specific binding to the target sequence region and the MEOW hybridizes to the binding reference region at a higher binding affinity than the labelled probe non-specific binding to the binding reference region;
optionally:
a positive control for the reference sequence;
a positive control for the target sequence; and
a mixed control comprising the reference sequence and the target sequence.

38. The kit of claim 36, wherein:
the forward primer is provided at a concentration of between 50 nM and 1100 nM;
the reverse primer is provided at a concentration of between 50 nM and 1100 nM;
the labelled probe is provided at a concentration of 20-800 nM;
the MEOW is provided at a concentration that is between 0.25× and 16× the concentration of the labelled probe; and/or
further comprising reagents for one or more of dPCR, ddPCR, qPCR, RT-dPCR, RT-ddPCR, RT-qPCR, or RT-PCR.

39. (canceled)

40. The kit of claim 36, wherein assay reagents are provided for a first reaction not comprising a MEOW and a second reaction comprising at least one MEOW.

41. The kit of claim 36, wherein the MEOW is provided to a wild-type reference sequence of a SARS-CoV-2 mutation to distinguish between at least one parental virus and at least one variant of the at least one parental virus, such as common Spike gene corresponding to residues HV69-70, R408, K417, L452, T478, N679, L704, Q954, and L981.

42. The kit of claim 36, wherein the reference sequence is:
a parental SARS-CoV-2 and the target sequence comprises a variant of SARS-CoV-2 selected from the group consisting of the Alpha variant, Beta variant, Gamma variant, Delta variant, Delta Plus variant, Mu variant, Lambda variant, Omicron variant, and Omicron subvariants.

43. The kit of claim 36, wherein the reference sequence is a proto-oncogene and the target sequence has a mutation that converts the proto-oncogene to an oncogene indicative of a higher risk of developing cancer or presence of cancer.

44. The kit of claim 43, wherein the proto-oncogene is KRAS and the target is a KRAS mutation at the 12^{th} or 13^{th} codon, including G12C, G12A, G12D, G12R, G12S, G12V, G13C, or G13D.

45. (canceled)

46. The kit of claim 36, wherein the MEOW is provided at a concentration so that one or more non-specific amplification population is optically indistinguishable from a negative population.

47. (canceled)

48. (canceled)

49. (canceled)

50. (canceled)


SEQUENCE LISTING (156 sequences)

SEQ ID NO: 1 - DNA, 17 bp, synthetic construct
  source 1..17: mol_type = other DNA
  modified_base 6: mod_base = OTHER; note = locked nucleic acid
  modified_base 9: mod_base = OTHER; note = locked nucleic acid
  modified_base 10: mod_base = OTHER; note = locked nucleic acid
  misc_feature 17: note = 3SpC3 (3' C3 spacer)
ctacgccaccagctcca

SEQ ID NO: 2 - DNA, 18 bp, synthetic construct
  source 1..18: mol_type = other DNA
  modified_base 17..18: mod_base = OTHER; note = locked nucleic acid
  misc_feature 18: note = 3SpC3 (3' C3 spacer)
tgtggtagttggagctgg

SEQ ID NO: 3 - DNA, 14 bp, synthetic construct
  source 1..14: mol_type = other DNA
  modified_base 10: mod_base = OTHER; note = LNA (locked nucleic acid)
  modified_base 13..14: mod_base = OTHER; note = LNA (locked nucleic acid)
  misc_feature 14: note = 3' Spacer (3SpC3)
ttgcctacgccacc

SEQ ID NO: 4 - DNA, 18 bp, synthetic construct
  source 1..18: mol_type = other DNA
  modified_base 9..10: mod_base = OTHER; note = LNA (locked nucleic acid)
  modified_base 13: mod_base = OTHER; note = LNA (locked nucleic acid)
  misc_feature 18: note = 3SpC3 (3' C3 spacer)
ttggagctggtggcgtag

SEQ ID NO: 5 - DNA, 18 bp, synthetic construct
  source 1..18: mol_type = other DNA
  modified_base 9..10: mod_base = OTHER; note = LNA (locked nucleic acid)
  modified_base 13: mod_base = OTHER; note = LNA (locked nucleic acid)
  misc_feature 18: note = CSpC6 (3' C6 spacer)
ttggagctggtggcgtag

SEQ ID NO: 6 - DNA, 18 bp, synthetic construct
  source 1..18: mol_type = other DNA
  misc_feature 1: note = 5SpC3 (5' C3 spacer)
  modified_base 9..10: mod_base = OTHER; note = LNA (locked nucleic acid)
  modified_base 13: mod_base = OTHER; note = LNA (locked nucleic acid)
  misc_feature 18: note = 3SpC3 (3' C3 spacer)
ttggagctggtggcgtag

SEQ ID NO: 7 - DNA, 18 bp, synthetic construct
  source 1..18: mol_type = other DNA
  misc_feature 1: note = 5SpC3 (5' C3 spacer)
  modified_base 8..9: mod_base = OTHER; note = LNA (locked nucleic acid)
  modified_base 13: mod_base = OTHER; note = LNA (locked nucleic acid)
  misc_feature 18: note = 3SpC3 (3' C3 spacer)
ttggagctggtggcgtag

SEQ ID NO: 8 - DNA, 19 bp, synthetic construct
  source 1..19: mol_type = other DNA
  modified_base 1: mod_base = OTHER; note = 52MOEr (5' 2'-O-methoxyethyl)
  modified_base 11..12: mod_base = OTHER; note = LNA (locked nucleic acid)
  modified_base 15: mod_base = OTHER; note = LNA (locked nucleic acid)
  modified_base 19: mod_base = OTHER; note = 3InvdT (3' Inverted deoxythymidine)
agttggagctggtggcgtt

SEQ ID NO: 9 - DNA, 20 bp, synthetic construct
  source 1..20: mol_type = other DNA
  modified_base 1: mod_base = OTHER; note = n represents an abasic site 5' 1',2'-Dideoxyribose (5dSp)
  modified_base 9..10: mod_base = OTHER; note = LNA (locked nucleic acid)
  modified_base 13: mod_base = OTHER; note = LNA (locked nucleic acid)
  misc_feature 20: note = 3C6 (3' hexanediol)
ntggagctggtggcgtaggc

SEQ ID NO: 10 - DNA, 20 bp, synthetic construct
  source 1..20: mol_type = other DNA
  modified_base 2: mod_base = OTHER; note = n represents an abasic site Int 1',2'-Dideoxyribose (iDsP)
  modified_base 9..10: mod_base = OTHER; note = LNA (locked nucleic acid)
  modified_base 13: mod_base = OTHER; note = LNA (locked nucleic acid)
  misc_feature 20: note = 3SpC3 (3' C3 spacer)
tnggagctggtggcgtaggc

SEQ ID NO: 11 - DNA, 19 bp, synthetic construct
  source 1..19: mol_type = other DNA
  modified_base 8..9: mod_base = OTHER; note = LNA (locked nucleic acid)
  misc_feature 19: note = 3SpC3 (3' blocker)
  modified_base 2..6: mod_base = OTHER; note = Phosphorothioate Bond
  modified_base 12: mod_base = OTHER; note = LNA (locked nucleic acid)
  modified_base 16..19: mod_base = OTHER; note = Phosphorothioate Bond
tggagctggtggcgtaggc

SEQ ID NO: 12 - DNA, 19 bp, synthetic construct
  source 1..19: mol_type = other DNA
  modified_base 1: mod_base = OTHER; note = 52MOEr (5' 2'-O-Methoxyethyl)
  modified_base 8..9: mod_base = OTHER; note = LNA (locked nucleic acid)
  modified_base 12: mod_base = OTHER; note = LNA (locked nucleic acid)
  modified_base 2..6: mod_base = OTHER; note = Phosphorothioate Bond
  modified_base 16..19: mod_base = OTHER; note = Phosphorothioate Bond
tggagctggtggcgtaggc

SEQ ID NO: 13 - DNA, 14 bp, synthetic construct
  source 1..14: mol_type = other DNA
  modified_base 1..5: mod_base = OTHER; note = LNA (locked nucleic acid)
  misc_feature 14: note = 3SpC3 (3' C3 spacer)
ggtggcgtaggcaa

SEQ ID NO: 14 - DNA, 25 bp, synthetic construct
  source 1..25: mol_type = other DNA
  modified_base 11..12: mod_base = OTHER; note = LNA (locked nucleic acid)
  modified_base 15: mod_base = OTHER; note = LNA (locked nucleic acid)
  misc_feature 25: note = 3SpC3 (3' C3 spacer)
  modified_base 2..6: mod_base = OTHER; note = Phosphorothioate Bond
  modified_base 21..25: mod_base = OTHER; note = Phosphorthioate Bond
agttggagctggtggcgtaggcaag

SEQ ID NO: 15 - DNA, 25 bp, synthetic construct
  source 1..25: mol_type = other DNA
  modified_base 11..12: mod_base = OTHER; note = LNA (locked nucleic acid)
  modified_base 15: mod_base = OTHER; note = LNA (locked nucleic acid)
  misc_feature 25: note = 3SpC3 (3' C3 spacer)
  modified_base 2..6: mod_base = OTHER; note = Phosphorothioate Bond
agttggagctggtggcgtaggcaag

SEQ ID NO: 16 - DNA, 25 bp, synthetic construct
  source 1..25: mol_type = other DNA
  modified_base 11..12: mod_base = OTHER; note = LNA (locked nucleic acid)
  modified_base 15: mod_base = OTHER; note = LNA (locked nucleic acid)
  misc_feature 25: note = 3SpC3 (3' C3 spacer)
  modified_base 2..5: mod_base = OTHER; note = Phosphorothioate Bond
agttggagctggtggcgtaggcaag

SEQ ID NO: 17 - DNA, 25 bp, synthetic construct
  source 1..25: mol_type = other DNA
  modified_base 11..12: mod_base = OTHER; note = LNA (locked nucleic acid)
  modified_base 15: mod_base = OTHER; note = LNA (locked nucleic acid)
  misc_feature 25: note = 3SpC3 (3' C3 spacer)
  modified_base 2..5: mod_base = OTHER; note = Phosphorothioate Bond
  modified_base 22..25: mod_base = OTHER; note = Phosphorothioate Bond
agttggagctggtggcgtaggcaag

SEQ ID NO: 18 - DNA, 16 bp, synthetic construct
  source 1..16: mol_type = other DNA
  modified_base 5..6: mod_base = OTHER; note = LNA (locked nucleic acid)
  modified_base 9: mod_base = OTHER; note = LNA (locked nucleic acid)
  misc_feature 16: note = 3SpC3 (3' C3 spacer)
  modified_base 2..5: mod_base = OTHER; note = Phosphorothioate Bond
  modified_base 13..16: mod_base = OTHER; note = Phosphorothioate Bond
agctggtggcgtaggc

SEQ ID NO: 19 - DNA, 16 bp, synthetic construct
  source 1..16: mol_type = other DNA
  modified_base 5..6: mod_base = OTHER; note = LNA (locked nucleic acid)
  modified_base 9: mod_base = OTHER; note = LNA (locked nucleic acid)
  misc_feature 16: note = 3SpC3 (3' C3 spacer)
  modified_base 2..5: mod_base = OTHER; note = Phosphorotihoate Bond
agctggtggcgtaggc

SEQ ID NO: 20 - DNA, 15 bp, synthetic construct
  source 1..15: mol_type = other DNA
  modified_base 1: mod_base = OTHER; note = 52MOEr (5' 2'-O-Methoxyethyl)
  modified_base 2: mod_base = OTHER; note = i2MOEr (int 2'-O-Methoxyethyl)
  modified_base 3: mod_base = OTHER; note = i2MOEr (int 2'-O-Methoxyethyl)
  modified_base 4..5: mod_base = OTHER; note = LNA (locked nucleic acid)
  modified_base 8: mod_base = OTHER; note = LNA (locked nucleic acid)
  misc_feature 15: note = 3SpC3 (3' C3 spacer)
gctggtggcgtaggc

SEQ ID NO: 21 - DNA, 15 bp, synthetic construct
  source 1..15: mol_type = other DNA
  modified_base 1: mod_base = OTHER; note = 52MOEr (5' 2'-O-methoxyethyl)
  modified_base 2: mod_base = OTHER; note = i2MOEr (int 2'-O-methoxyethyl)
  modified_base 3: mod_base = OTHER; note = i2MOEr (int 2'-O-Methoxyethyl)
  modified_base 4..5: mod_base = OTHER; note = LNA (locked nucleic acid)
  modified_base 8: mod_base = OTHER; note = LNA (locked nucleic acid)
  misc_feature 15: note = 3SpC3 (3' C3 spacer)
  modified_base 2..4: mod_base = OTHER; note = Phosphorothioate Bond
gctggtggcgtaggc

SEQ ID NO: 22 - DNA, 15 bp, synthetic construct
  source 1..15: mol_type = other DNA
  modified_base 1: mod_base = OTHER; note = 52MOEr (5' 2'-O-Methoxyethyl)
  modified_base 2: mod_base = OTHER; note = i2MOEr (int 2'-O-Methoxyethyl)
  modified_base 3: mod_base = OTHER; note = i2MOEr (int 2'-O-Methoxyethyl)
  modified_base 4..5: mod_base = OTHER; note = LNA (locked nucleic acid)
  modified_base 8: mod_base = OTHER; note = LNA (locked nucleic acid)
  misc_feature 15: note = 3SpC3 (3' C3 spacer)
  modified_base 2..4: mod_base = OTHER; note = Phosphorothioate Bond
gctggtggcgtaggc

SEQ ID NO: 23 - DNA, 15 bp, synthetic construct
  source 1..15: mol_type = other DNA
  modified_base 1..3: mod_base = OTHER; note = LNA (locked nucleic acid)
  modified_base 6: mod_base = OTHER; note = LNA (locked nucleic acid)
  misc_feature 15: note = 3SpC3 (3' C3 spacer)
tggtggcgtaggcaa

SEQ ID NO: 24 - DNA, 19 bp, synthetic construct
  source 1..19: mol_type = other DNA
  modified_base 1..3: mod_base = OTHER; note = LNA (locked nucleic acid)
  modified_base 8..9: mod_base = OTHER; note = LNA (locked nucleic acid)
  modified_base 12: mod_base = OTHER; note = LNA (locked nucleic acid)
  misc_feature 19: note = 3SpC3 (3' C3 spacer)
tggagctggtggcgtaggc

SEQ ID NO: 25 - DNA, 14 bp, synthetic construct
  source 1..14: mol_type = other DNA
  modified_base 1..4: mod_base = OTHER; note = LNA (locked nucleic acid)
  misc_feature 14: note = 3SpC3 (3' C3 spacer)
ggtggcgtaggcaa

SEQ ID NO: 26 - DNA, 20 bp, synthetic construct
  source 1..20: mol_type = other DNA
  modified_base 1: mod_base = OTHER; note = n represents an abasic site 5' 1',2'-Dideoxyribose (5dSp)
  modified_base 6..7: mod_base = OTHER; note = LNA (locked nucleic acid)
  modified_base 9..10: mod_base = OTHER; note = LNA (locked nucleic acid)
  misc_feature 20: note = 3SpC3 (3' C3 spacer)
nagctggtggcgtaggcaag

SEQ ID NO: 27 - DNA, 21 bp, synthetic construct
  source 1..21: mol_type = other DNA
  modified_base 1: mod_base = OTHER; note = n represents an abasic site 5' 1',2'-Dideoxyribose (5dSp)
  modified_base 6..7: mod_base = OTHER; note = LNA (locked nucleic acid)
  misc_feature 21: note = 3SpC3 (3' C3 spacer)
nagctggtggcgtaggcaaga

SEQ ID NO: 28 - DNA, 21 bp, synthetic construct
  source 1..21: mol_type = other DNA
  modified_base 1: mod_base = OTHER; note = n represents an abasic site 5' 1',2'-Dideoxyribose (5dSp)
  modified_base 6..7: mod_base = OTHER; note = LNA (locked nucleic acid)
  modified_base 9: mod_base = OTHER; note = LNA (locked nucleic acid)
  misc_feature 21: note = 3SpC3 (3' C3 spacer)
nagctggtggcgtaggcaaga

SEQ ID NO: 29 - DNA, 19 bp, synthetic construct
  source 1..19: mol_type = other DNA
  modified_base 4: mod_base = OTHER; note = OXO (8-oxoadenine)
  misc_feature 19: note = 3SpC3 (3' C3 spacer)
tggagctggtggcgtaggc

SEQ ID NO: 30 - DNA, 20 bp, synthetic construct
  source 1..20: mol_type = other DNA
  modified_base 1: mod_base = OTHER; note = n represents an abasic site 5' 1',2'-Dideoxyribose (5dSp)
  modified_base 9..10: mod_base = OTHER; note = LNA (locked nucleic acid)
  modified_base 13: mod_base = OTHER; note = LNA (locked nucleic acid)
ntggagctggtggcgtaggc

SEQ ID NO: 31 - DNA, 14 bp, synthetic construct
  source 1..14: mol_type = other DNA
  modified_base 1..5: mod_base = OTHER; note = LNA (locked nucleic acid)
ggtggcgtaggcaa

SEQ ID NO: 32 - DNA, 20 bp, synthetic construct
  source 1..20: mol_type = other DNA
  modified_base 1: mod_base = OTHER; note = n represents an abasic site 5' 1',2'-Dideoxyribose (5dSp)
  modified_base 6..7: mod_base = OTHER; note = LNA (locked nucleic acid)
  modified_base 9..10: mod_base = OTHER; note = LNA (locked nucleic acid)
  misc_feature 20: note = 3SpC3 (3' C3 spacer)
nagctggtggcgtaggcaag

SEQ ID NO: 33 - DNA, 13 bp, synthetic construct
  source 1..13: mol_type = other DNA
  modified_base 1..5: mod_base = OTHER; note = LNA (locked nucleic acid)
  misc_feature 13: note = 3SpC3 (3' C3 blocker)
ggcgtaggcaaga

SEQ ID NO: 34 - DNA, 19 bp, synthetic construct
  source 1..19: mol_type = other DNA
  modified_base 1: mod_base = OTHER; note = n represents an abasic site 5' 1',2'-Dideoxyribose (5dSp)
  modified_base 4..5: mod_base = OTHER; note = LNA (locked nucleic acid)
  modified_base 7..8: mod_base = OTHER; note = LNA (locked nucleic acid)
  misc_feature 19: note = 3SpC3 (3' C3 spacer)
nctggtggcgtaggcaaga

SEQ ID NO: 35 - DNA, 19 bp, synthetic construct
  source 1..19: mol_type = other DNA
  modified_base 1: mod_base = OTHER; note = n represents an abasic site 5' 1',2'-Dideoxyribose (5dSp)
  modified_base 4..5: mod_base = OTHER; note = LNA (locked nucleic acid)
  modified_base 7..8: mod_base = OTHER; note = LNA (locked nucleic acid)
  misc_feature 19: note = 3SpC3 (3' C3 spacer)
ncgccaccagctccaacta

SEQ ID NO: 36 - DNA, 15 bp, synthetic construct
  source 1..15: mol_type = other DNA
  modified_base 1..5: mod_base = OTHER; note = LNA (locked nucleic acid)
  misc_feature 15: note = 3SpC3 (3' C3 spacer)
ggtggcgtaggcaag

SEQ ID NO: 37 - DNA, 18 bp, synthetic construct
  source 1..18: mol_type = other DNA
  modified_base 1..5: mod_base = OTHER; note = LNA (locked nucleic acid)
  misc_feature 18: note = 3SpC3 (3' C3 spacer)
ccaccagctccaactacc

SEQ ID NO: 38 - DNA, 21 bp, synthetic construct
  source 1..21: mol_type = other DNA
  modified_base 1: mod_base = OTHER; note = n represents an abasic site 5' 1',2'-Dideoxyribose (5dSp)
  modified_base 6..7: mod_base = OTHER; note = LNA (locked nucleic acid)
  misc_feature 21: note = 3SpC3 (3' C3 spacer)
nagctggtggcgtaggcaaga

SEQ ID NO: 39 - DNA, 21 bp, synthetic construct
  source 1..21: mol_type = other DNA
  modified_base 1: mod_base = OTHER; note = n represents an abasic site 5' 1',2'-Dideoxyribose (5dSp)
  modified_base 6..7: mod_base = OTHER; note = LNA (locked nucleic acid)
  modified_base 9: mod_base = OTHER; note = LNA (locked nucleic acid)
  misc_feature 21: note = 3SpC3 (3' C3 spacer)
nagctggtggcgtaggcaaga

SEQ ID NO: 40 - DNA, 14 bp, synthetic construct
  source 1..14: mol_type = other DNA
  modified_base 1..4: mod_base = OTHER; note = LNA (locked nucleic acid)
  misc_feature 14: note = 3SpC3 (3' C3 spacer)
ggtggcgtaggcaa

SEQ ID NO: 41 - DNA, 15 bp, synthetic construct
  source 1..15: mol_type = other DNA
  modified_base 1..3: mod_base = OTHER; note = LNA (locked nucleic acid)
  modified_base 6: mod_base = OTHER; note = LNA (locked nucleic acid)
  misc_feature 15: note = 3SpC3 (3' C3 spacer)
gccaccagctccaac

SEQ ID NO: 42 - DNA, 20 bp, synthetic construct
  source 1..20: mol_type = other DNA
  modified_base 1: mod_base = OTHER; note = n represents an abasic site 5' 1',2'-Dideoxyribose (5dSp)
  modified_base 8: mod_base = OTHER; note = LNA (locked nucleic acid)
  modified_base 11..12: mod_base = OTHER; note = LNA (locked nucleic acid)
  misc_feature 20: note = 3SpC3 (3' C3 spacer)
ncctacgccaccagctccaa

SEQ ID NO: 43 - DNA, 15 bp, synthetic construct
  source 1..15: mol_type = other DNA
  modified_base 1: mod_base = OTHER; note = 52MOEr (5' 2-O-Methoxyethyl)
  modified_base 2..3: mod_base = OTHER; note = i2MOEr (2'-O-Methoxyethyl)
  modified_base 4: mod_base = OTHER; note = LNA (locked nucleic acid)
  modified_base 7..8: mod_base = OTHER; note = LNA (locked nucleic acid)
  misc_feature 15: note = 3SpC3 (3' C3 spacer)
acgccaccagctcca

SEQ ID NO: 44 - DNA, 15 bp, synthetic construct
  source 1..15: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  modified_base 4..5: mod_base = OTHER; note = LNA (locked nucleic acid)
  modified_base 8: mod_base = OTHER; note = LNA (locked nucleic acid)
  misc_feature 15: note = 3SpC3 (3' C3 spacer)
acgccaccagctcca

SEQ ID NO: 45 - DNA, 20 bp, synthetic construct
  source 1..20: mol_type = other DNA
  modified_base 1: mod_base = OTHER; note = n represents an abasic site 5' 1',2'-Dideoxyribose (5dSp)
  modified_base 10: mod_base = OTHER; note = LNA (locked nucleic acid)
  misc_feature 20: note = 3SpC3 (3' C3 spacer)
ngctcccagacctgctgaaa

SEQ ID NO: 46 - DNA, 19 bp, synthetic construct
  source 1..19: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  modified_base 9: mod_base = OTHER; note = LNA (locked nucleic acid)
  misc_feature 19: note = 3SpC3 (3' C3 spacer)
gctcccagacctgctgaaa

SEQ ID NO: 47 - DNA, 16 bp, synthetic construct
  source 1..16: mol_type = other DNA
  modified_base 1: mod_base = OTHER; note = n represents an abasic site 5' 1',2'-Dideoxyribose (5dSp)
  modified_base 14: mod_base = OTHER; note = LNA (locked nucleic acid)
  misc_feature 16: note = 3SpC3 (3' C3 spacer)
nagcggctcccagacc

SEQ ID NO: 48 - DNA, 21 bp, synthetic construct
  source 1..21: mol_type = other DNA
  modified_base 1: mod_base = OTHER; note = n represents an abasic site 5' 1',2'-Dideoxyribose (5dSp)
  modified_base 4: mod_base = OTHER; note = LNA (locked nucleic acid)
  misc_feature 21: note = 3SpC3 (3' C3 spacer)
nagacctgctgaaaatgactg

SEQ ID NO: 49 - DNA, 21 bp, synthetic construct
  source 1..21: mol_type = other DNA
  modified_base 1: mod_base = OTHER; note = n represents an abasic site 5' 1',2'-Dideoxyribose (5dSp)
  modified_base 12: mod_base = OTHER; note = LNA (locked nucleic acid)
  misc_feature 21: note = 3SpC3 (3' C3 spacer)
nggatcatattggtccacaaa

SEQ ID NO: 50 - DNA, 26 bp, synthetic construct
  source 1..26: mol_type = other DNA
  modified_base 1: mod_base = OTHER; note = n represents an abasic site 5' 1',2'-Dideoxyribose (5dSp)
  modified_base 22: mod_base = OTHER; note = LNA (locked nucleic acid)
  misc_feature 26: note = 3SpC3 (3' C3 spacer)
nctctattgttggatcatattggtcc

SEQ ID NO: 51 - DNA, 16 bp, synthetic construct
  source 1..16: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  modified_base 12..13: mod_base = OTHER; note = LNA (locked nucleic acid)
  modified_base 15..16: mod_base = OTHER; note = LNA (locked nucleic acid)
  misc_feature 16: note = 3SpC3 (3' C3 spacer)
tcttgcctacgccacc

SEQ ID NO: 52 - DNA, 17 bp, synthetic construct
  source 1..17: mol_type = other DNA
  modified_base 1: mod_base = OTHER; note = n represents an abasic site 5' 1',2'-Dideoxyribose (5dSp)
  modified_base 13..14: mod_base = OTHER; note = LNA (locked nucleic acid)
  modified_base 16..17: mod_base = OTHER; note = LNA (locked nucleic acid)
  misc_feature 17: note = 3SpC3 (3' C3 spacer)
ntcttgcctacgccacc

SEQ ID NO: 53 - DNA, 20 bp, synthetic construct
  source 1..20: mol_type = other DNA
  modified_base 1..4: mod_base = OTHER; note = LNA (locked nucleic acid)
  misc_feature 20: note = 3SpC3 (3' C3 spacer)
ggtggcgtaggcaagagtgc

SEQ ID NO: 54 - DNA, 17 bp, synthetic construct
  source 1..17: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  modified_base 7..8: mod_base = OTHER; note = LNA (locked nucleic acid)
  modified_base 11: mod_base = OTHER; note = LNA (locked nucleic acid)
  misc_feature 17: note = 3SpC3 (3' C3 spacer)
cctacgccaccagctcc

SEQ ID NO: 55 - DNA, 14 bp, synthetic construct
  source 1..14: mol_type = other DNA
  modified_base 1..2: mod_base = OTHER; note = LNA (locked nucleic acid)
  modified_base 4..5: mod_base = OTHER; note = LNA (locked nucleic acid)
  misc_feature 14: note = 3SpC3 (3' C3 spacer)
ggtggcgtaggcaa

SEQ ID NO: 56 - DNA, 15 bp, synthetic construct
  source 1..15: mol_type = other DNA
  modified_base 6..7: mod_base = OTHER; note = LNA (locked nucleic acid)
  modified_base 10: mod_base = OTHER; note = LNA (locked nucleic acid)
  misc_feature 15: note = 3SpC3 (3' C3 spacer)
  modified_base 1: mod_base = gm
gagctggtggcgtag

SEQ ID NO: 57 - DNA, 15 bp, synthetic construct
  source 1..15: mol_type = other DNA
  modified_base 6..7: mod_base = OTHER; note = LNA (locked nucleic acid)
  modified_base 10: mod_base = OTHER; note = LNA (locked nucleic acid)
  misc_feature 15: note = 3SpC3 (3' C3 spacer)
  modified_base 1: mod_base = gm
  modified_base 2: mod_base = OTHER; note = 2'-O-methyladenosine
gagctggtggcgtag

SEQ ID NO: 58 - DNA, 14 bp, synthetic construct
  source 1..14: mol_type = other DNA
  modified_base 6..7: mod_base = OTHER; note = LNA (locked nucleic acid)
  modified_base 10: mod_base = OTHER; note = LNA (locked nucleic acid)
  misc_feature 14: note = 3SpC3 (3' C3 spacer)
  modified_base 1: mod_base = gm
  modified_base 2: mod_base = OTHER; note = 2'-O-methyladenosine
  modified_base 3: mod_base = gm
gagctggtggcgta

SEQ ID NO: 59 - DNA, 14 bp, synthetic construct
  source 1..14: mol_type = other DNA
  modified_base 6..7: mod_base = OTHER; note = LNA (locked nucleic acid)
  modified_base 10: mod_base = OTHER; note = LNA (locked nucleic acid)
  misc_feature 14: note = 3SpC3 (3' C3 spacer)
  modified_base 1: mod_base = gm
  modified_base 2: mod_base = OTHER; note = 2'-O-methyladenosine
  modified_base 3: mod_base = gm
  modified_base 4: mod_base = cm
gagctggtggcgta

SEQ ID NO: 60 - DNA, 15 bp, synthetic construct
  source 1..15: mol_type = other DNA
  modified_base 7..8: mod_base = OTHER; note = LNA (locked nucleic acid)
  modified_base 11: mod_base = OTHER; note = LNA (locked nucleic acid)
  misc_feature 15: note = 3SpC3 (3' C3 spacer)
  modified_base 1: mod_base = gm
  modified_base 2: mod_base = gm
  modified_base 3: mod_base = OTHER; note = 2'-O-methyladenosine
  modified_base 4: mod_base = gm
  modified_base 5: mod_base = cm
ggagctggtggcgta

SEQ ID NO: 61 - DNA, 15 bp, synthetic construct
  source 1..15: mol_type = other DNA
  modified_base 7..8: mod_base = OTHER; note = LNA (locked nucleic acid)
  modified_base 11: mod_base = OTHER; note = LNA (locked nucleic acid)
  misc_feature 15: note = 3SpC3 (3' C3 spacer)
  modified_base 1..2: mod_base = gm
  modified_base 3: mod_base = OTHER; note = 2'-O-methyladenosine
  modified_base 4: mod_base = gm
  modified_base 5: mod_base = cm
  modified_base 6: mod_base = OTHER; note = 2'-O-methylthymidine
  misc_feature 1..6: note = RNA
  misc_feature 7..15: note = DNA
ggagctggtggcgta

SEQ ID NO: 62 - DNA, 20 bp, synthetic construct
  source 1..20: mol_type = other DNA
  misc_feature 20: note = 3SpC3 (3' C3 spacer)
  modified_base 1..2: mod_base = gm
  modified_base 3: mod_base = um
  modified_base 4: mod_base = gm
  misc_feature 1..3: note = RNA
  misc_feature 4..20: note = DNA
ggtggcgtaggcaagagtgc

SEQ ID NO: 63 - DNA, 14 bp, synthetic construct
  source 1..14: mol_type = other DNA
  modified_base 5: mod_base = OTHER; note = LNA (locked nucleic acid)
  modified_base 8..9: mod_base = OTHER; note = LNA (locked nucleic acid)
  misc_feature 14: note = 3SpC3 (3' C3 spacer)
  modified_base 1: mod_base = um
  modified_base 2: mod_base = OTHER; note = 2'-O-methyladenosine
  modified_base 3: mod_base = cm
  modified_base 4: mod_base = gm
  misc_feature 1: note = RNA
  misc_feature 2..14: note = DNA
tacgccaccagctc

SEQ ID NO: 64 - DNA, 15 bp, synthetic construct
  source 1..15: mol_type = other DNA
  modified_base 1..5: mod_base = OTHER; note = LNA (locked nucleic acid)
aagaacgggcagatc

SEQ ID NO: 65 - DNA, 19 bp, synthetic construct
  source 1..19: mol_type = other DNA
  modified_base 1: mod_base = OTHER; note = 56-ROXN (5' ROX (carboxy-N-rhodamine))
  modified_base 19: mod_base = OTHER; note = 3' Iowa Black RQ
cgccttccatggagacgca

SEQ ID NO: 66 - DNA, 15 bp, synthetic construct
  source 1..15: mol_type = other DNA
  modified_base 1..5: mod_base = OTHER; note = LNA (locked nucleic acid)
acctgagagccagaa

SEQ ID NO: 67 - DNA, 20 bp, synthetic construct
  source 1..20: mol_type = other DNA
  modified_base 1: mod_base = OTHER; note = 5Cy55 (5' sulfo-cyanine5.5)
  modified_base 8: mod_base = OTHER; note = LNA (locked nucleic acid)
  modified_base 10: mod_base = OTHER; note = LNA (locked nucleic acid)
  modified_base 20: mod_base = OTHER; note = 3IAbRQSp (3' Iowa Black RQ)
ccaaggatccaacgaccaag

SEQ ID NO: 68 - DNA, 15 bp, synthetic construct
  source 1..15: mol_type = other DNA
  modified_base 1..5: mod_base = OTHER; note = LNA (locked nucleic acid)
tgaccaactcgtgtg

SEQ ID NO: 69 - DNA, 21 bp, synthetic construct
  source 1..21: mol_type = other DNA
  modified_base 1: mod_base = OTHER; note = 56-FAM (5' 6-fluorescein)
  modified_base 12: mod_base = OTHER; note = LNA (locked nucleic acid)
  modified_base 21: mod_base = OTHER; note = 3IABkFQ (3' Iowa Black FQ)
cagactgtccacagcattccg

SEQ ID NO: 70 - DNA, 19 bp, synthetic construct
  source 1..19: mol_type = other DNA
  modified_base 1..5: mod_base = OTHER; note = LNA (locked nucleic acid)
caagagagagaggtccaag

SEQ ID NO: 71 - DNA, 18 bp, synthetic construct
  source 1..18: mol_type = other DNA
  modified_base 1: mod_base = OTHER; note = 5Cy5 (sulfo-cyanine5)
  modified_base 3: mod_base = OTHER; note = LNA (locked nucleic acid)
  modified_base 12: mod_base = OTHER; note = LNA (locked nucleic acid)
  modified_base 18: mod_base = OTHER; note = 3IAbRQSp (3' Iowa Black RQ)
ctacatcgtgcgccagtg

SEQ ID NO: 72 - DNA, 23 bp, synthetic construct
  source 1..23: mol_type = other DNA
gcctaagacccggagcttttcac

SEQ ID NO: 73 - DNA, 26 bp, synthetic construct
  source 1..26: mol_type = other DNA
  modified_base 2..4: mod_base = OTHER; note = LNA (locked nucleic acid)
tatcatcactgagttcatgacctacg

SEQ ID NO: 74 - DNA, 19 bp, synthetic construct
  source 1..19: mol_type = other DNA
  modified_base 1: mod_base = OTHER; note = 5HEX (5' hexachlorofluorescein)
  modified_base 8: mod_base = OTHER; note = LNA (locked nucleic acid)
  modified_base 11: mod_base = OTHER; note = LNA (locked nucleic acid)
  modified_base 19: mod_base = OTHER; note = LNA (locked nucleic acid)
cgtggtgctgctgtacatg

SEQ ID NO: 75 - DNA, 21 bp, synthetic construct
  source 1..21: mol_type = other DNA
ctgtcatcaacctgctcaggc

SEQ ID NO: 76 - DNA, 25 bp, synthetic construct
  source 1..25: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  misc_feature 25: note = 3SpC3 (3' C3 Spacer)
ccttcgtatctcagcgagatggacc

SEQ ID NO: 77 - DNA, 25 bp, synthetic construct
  source 1..25: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  misc_feature 25: note = 3SpC3 (3' C3 Spacer)
tccttgtggatctcgtagagctcag

SEQ ID NO: 78 - DNA, 26 bp, synthetic construct
  source 1..26: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  misc_feature 26: note = 3SpC3 (3' C3 Spacer)
ctccagttcatccaccatctggaagc

SEQ ID NO: 79 - DNA, 24 bp, synthetic construct
  source 1..24: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  misc_feature 24: note = 3SpC3 (3' C3 Spacer)
gggatcttcgtcttgttgtaacac

SEQ ID NO: 80 - DNA, 21 bp, synthetic construct
  source 1..21: mol_type = other DNA
rgcyggtatytcyaaggaygg

SEQ ID NO: 81 - DNA, 21 bp, synthetic construct
  source 1..21: mol_type = other DNA
gtgrtgcatytcracrgaytt

SEQ ID NO: 82 - DNA, 21 bp, synthetic construct
  source 1..21: mol_type = other DNA
gtggtgcatttcaacactctt

SEQ ID NO: 83 - DNA, 18 bp, synthetic construct
  source 1..18: mol_type = other DNA
  modified_base 1: mod_base = OTHER; note = 56-FAM (5' 6-fluorescein)
  modified_base 5: mod_base = OTHER; note = LNA (locked nucleic acid)
  modified_base 8: mod_base = OTHER; note = LNA (locked nucleic acid)
  modified_base 16..17: mod_base = OTHER; note = LNA (locked nucleic acid)
  modified_base 18: mod_base = OTHER; note = 3IABkFQ (3' Iowa Black FQ)
tggmayggtgacaacatg

SEQ ID NO: 84 - DNA, 18 bp, synthetic construct
  source 1..18: mol_type = other DNA
  modified_base 1: mod_base = OTHER; note = 56-FAM (5' 6-fluorescein)
  modified_base 7..8: mod_base = OTHER; note = LNA (locked nucleic acid)
  modified_base 13: mod_base = OTHER; note = LNA (locked nucleic acid)
  modified_base 16: mod_base = OTHER; note = LNA (locked nucleic acid)
  modified_base 18: mod_base = OTHER; note = 3IABkFQ (3' Iowa Black FQ)
ttcaacggygacaacatg

SEQ ID NO: 85 - DNA, 18 bp, synthetic construct
  source 1..18: mol_type = other DNA
  modified_base 1: mod_base = OTHER; note = 56-FAM (5' 6-fluoroscein)
  modified_base 5: mod_base = OTHER; note = LNA (locked nucleic acid)
  modified_base 8: mod_base = OTHER; note = LNA (locked nucleic acid)
  modified_base 17: mod_base = OTHER; note = LNA (locked nucleic acid)
  modified_base 18: mod_base = OTHER; note = 3IABkFQ (3'Iowa Black FQ)
tksmacggtgayaacatg

SEQ ID NO: 86 - DNA, 18 bp, synthetic construct
  source 1..18: mol_type = other DNA
  modified_base 1: mod_base = OTHER; note = 56-FAM (5' 6-fluorescein)
  modified_base 8: mod_base = OTHER; note = LNA (locked nucleic acid)
  modified_base 10..11: mod_base = OTHER; note = LNA (locked nucleic acid)
  modified_base 18: mod_base = OTHER; note = 3IABkFQ (3' Iowa Black FQ)
ttcmacgghgacaayatg

SEQ ID NO: 87 - DNA, 18 bp, synthetic construct
  source 1..18: mol_type = other DNA
  modified_base 1: mod_base = OTHER; note = 56-FAM (5' 6-fluorescein)
  modified_base 9: mod_base = OTHER; note = LNA (locked nucleic acid)
  modified_base 18: mod_base = OTHER; note = 3IABkFQ (3' Iowa Black FQ)
tggcacggagacaagtga

SEQ ID NO: 88 - DNA, 20 bp, synthetic construct
  source 1..20: mol_type = other DNA
aygaacaaagcatcccacct

SEQ ID NO: 89 - DNA, 22 bp, synthetic construct
  source 1..22: mol_type = other DNA
tggagcatacacagtacayvcr

SEQ ID NO: 90 - DNA, 18 bp, synthetic construct
  source 1..18: mol_type = other DNA
  modified_base 1: mod_base = OTHER; note = ZEN (Zen internal quencher)
  modified_base 18: mod_base = OTHER; note = 3IABkFQ (3' Iowa Black FQ)
catcrtcatwaatgatct

SEQ ID NO: 91 - DNA, 18 bp, synthetic construct
  source 1..18: mol_type = other DNA
  modified_base 4: mod_base = OTHER; note = LNA (locked nucleic acid)
  modified_base 6: mod_base = OTHER; note = LNA (locked nucleic acid)
  misc_feature 18: note = 3SpC3 (3' C3 Spacer)
ttcgagggtgacaacatg

SEQ ID NO: 92 - DNA, 21 bp, synthetic construct
  source 1..21: mol_type = other DNA
  modified_base 1..5: mod_base = OTHER; note = LNA (locked nucleic acid)
  misc_feature 21: note = 3SpC3 (3' C3 spacer)
gatttgctgttgtaacaaggt

SEQ ID NO: 93 - DNA, 21 bp, synthetic construct
  source 1..21: mol_type = other DNA
tgtggatttgaccctccatga

SEQ ID NO: 94 - DNA, 23 bp, synthetic construct
  source 1..23: mol_type = other DNA
ccaatcatcaggatctctagcca

SEQ ID NO: 95 - DNA, 21 bp, synthetic construct
  source 1..21: mol_type = other DNA
  modified_base 1: mod_base = OTHER; note = 56-ROXN (5' 6-carboxy-X-rhodamine)
  modified_base 12: mod_base = OTHER; note = LNA (locked nucleic acid)
  modified_base 21: mod_base = OTHER; note = 3IAbRQSp (3' Iowa Black RQ)
tccaccttctacaatgtgcct

SEQ ID NO: 96 - DNA, 20 bp, synthetic construct
  source 1..20: mol_type = other DNA
tctctgcgcattcaggagtg

SEQ ID NO: 97 - DNA, 24 bp, synthetic construct
  source 1..24: mol_type = other DNA
gtggatatggtccttctcttccag

SEQ ID NO: 98 - DNA, 19 bp, synthetic construct
  source 1..19: mol_type = other DNA
  modified_base 1: mod_base = OTHER; note = 5Cy55 (Sulfo-Cyanine5.5)
  modified_base 11: mod_base = OTHER; note = LNA (locked nucleic acid)
  modified_base 19: mod_base = OTHER; note = 3IAbRQSp (3' Iowa Black RQ)
cacatttctgcccagcacc

SEQ ID NO: 99 - DNA, 21 bp, synthetic construct
  source 1..21: mol_type = other DNA
ggcatggagcatctgtacagc

SEQ ID NO: 100 - DNA, 17 bp, synthetic construct
  source 1..17: mol_type = other DNA
tgggcgtccagcatctc

SEQ ID NO: 101 - DNA, 17 bp, synthetic construct
  source 1..17: mol_type = other DNA
  modified_base 1: mod_base = OTHER; note = 5Cy5 (5' Sulfo-Cyanine5)
  modified_base 10: mod_base = OTHER; note = LNA (locked nucleic acid)
  modified_base 17: mod_base = OTHER; note = 3IAbRQSp (3' Iowa Black RQ)
tggtgccccgctatgac

SEQ ID NO: 102 - DNA, 18 bp, synthetic construct
  source 1..18: mol_type = other DNA
  modified_base 1: mod_base = OTHER; note = 5Cy5 (5' Sulfo-Cyanine5)
  modified_base 10: mod_base = OTHER; note = LNA (locked nucleic acid)
  modified_base 18: mod_base = OTHER; note = 3IAbRQSp (3' Iowa Black RQ)
tggtgccccactatgacc

SEQ ID NO: 103 - DNA, 17 bp, synthetic construct
  source 1..17: mol_type = other DNA
  modified_base 1: mod_base = OTHER; note = 5Cy5 (5' Sulfo-cyanine5)
  modified_base 10: mod_base = OTHER; note = LNA (locked nucleic acid)
  modified_base 17: mod_base = OTHER; note = 3IAbRQSp (3' Iowa Black RQ)
tggtgcccccctatgac

SEQ ID NO: 104 - DNA, 17 bp, synthetic construct
  source 1..17: mol_type = other DNA
  modified_base 1: mod_base = OTHER; note = 5HEX (5' hexachlorofluorescein)
  modified_base 10: mod_base = OTHER; note = LNA (locked nucleic acid)
  modified_base 17: mod_base = OTHER; note = 3IABkFQ (3' Iowa Black FQ)
tgcccctctgtgacctg

SEQ ID NO: 105 - DNA, 19 bp, synthetic construct
  source 1..19: mol_type = other DNA
  modified_base 1: mod_base = OTHER; note = 5HEX (5' hexachlorofluorescein)
  modified_base 13: mod_base = OTHER; note = LNA (locked nucleic acid)
  modified_base 19: mod_base = OTHER; note = 3IABkFQ (3' Iowa Black FQ)
tggtgcccctctctgacct

SEQ ID NO: 106 - DNA, 18 bp, synthetic construct
  source 1..18: mol_type = other DNA
  modified_base 1: mod_base = OTHER; note = 5HEX (5' hexachlorofluorescein)
  modified_base 9: mod_base = OTHER; note = LNA (locked nucleic acid)
  modified_base 18: mod_base = OTHER; note = 3IABkFQ (3' Iowa Black FQ)
tgcccctcaatgacctgc

SEQ ID NO: 107 - DNA, 18 bp, synthetic construct
  source 1..18: mol_type = other DNA
  modified_base 1: mod_base = OTHER; note = 56-FAM (5' 6-fluorescein)
  modified_base 9: mod_base = OTHER; note = LNA (locked nucleic acid)
  modified_base 18: mod_base = OTHER; note = 3IABkFQ (3' Iowa Black FQ)
cctctatggcctgctgct

SEQ ID NO: 108 - DNA, 21 bp, synthetic construct
  source 1..21: mol_type = other DNA
  modified_base 12: mod_base = OTHER; note = LNA (locked nucleic acid)
  misc_feature 21: note = 3SpC3 (3' C3 spacer)
tccaccttctagaatgtgcct

SEQ ID NO: 109 - DNA, 23 bp, synthetic construct
  source 1..23: mol_type = other DNA
  modified_base 13: mod_base = OTHER; note = LNA (locked nucleic acid)
  misc_feature 23: note = 3SpC3 (3' C3 spacer)
tacacatttctgtccagcaccct

SEQ ID NO: 110 - DNA, 19 bp, synthetic construct
  source 1..19: mol_type = other DNA
  modified_base 1: mod_base = OTHER; note = 52MOEr (5' 2'-O-Methoxyethyl)
  modified_base 2..3: mod_base = OTHER; note = i2MOEr (2'-O-Methoxyethyl)
  modified_base 4: mod_base = OTHER; note = LNA (locked nucleic acid)
  modified_base 6..7: mod_base = OTHER; note = LNA (locked nucleic acid)
  modified_base 10: mod_base = OTHER; note = LNA (locked nucleic acid)
  misc_feature 19: note = 3SpC3 (3' C3 spacer)
ccctctatgacctgctgct

SEQ ID NO: 111 - DNA, 19 bp, synthetic construct
  source 1..19: mol_type = other DNA
  modified_base 4: mod_base = OTHER; note = LNA (locked nucleic acid)
  modified_base 6..7: mod_base = OTHER; note = LNA (locked nucleic acid)
  modified_base 10: mod_base = OTHER; note = LNA (locked nucleic acid)
  misc_feature 19: note = 3SpC3 (3' C3 spacer)
ccctctatgacctgctgct

SEQ ID NO: 112 - DNA, 21 bp, synthetic construct
  source 1..21: mol_type = other DNA
  misc_feature 21: note = 3SpC3 (3' C3 spacer)
gcccctctatgacctgctgct

SEQ ID NO: 113 - DNA, 23 bp, synthetic construct
  source 1..23: mol_type = other DNA
  modified_base 15: mod_base = OTHER; note = LNA (locked nucleic acid)
  modified_base 20: mod_base = OTHER; note = LNA (locked nucleic acid)
gatgttgaactatgcaaagagac

SEQ ID NO: 114 - DNA, 25 bp, synthetic construct
  source 1..25: mol_type = other DNA
ggacagccagatatcaactgttaca

SEQ ID NO: 115 - DNA, 21 bp, synthetic construct
  source 1..21: mol_type = other DNA
  modified_base 1: mod_base = OTHER; note = 56-FAM (5' 6-fluorescein)
  modified_base 21: mod_base = OTHER; note = 3IABkFQ (3' Iowa Black FQ)
tgtggttaaggaaccacaaca

SEQ ID NO: 116 - DNA, 28 bp, synthetic construct
  source 1..28: mol_type = other DNA
  modified_base 6..9: mod_base = OTHER; note = LNA (locked nucleic acid)
  misc_feature 28: note = 3SpC3 (3' C3 spacer)
ccactgccagagatcttgaatagcctgg

SEQ ID NO: 117 - DNA, 22 bp, synthetic construct
  source 1..22: mol_type = other DNA
cccatgacacaatccagcttta

SEQ ID NO: 118 - DNA, 22 bp, synthetic construct
  source 1..22: mol_type = other DNA
aagcatcatggcagtagttctc

SEQ ID NO: 119 - DNA, 20 bp, synthetic construct
  source 1..20: mol_type = other DNA
aggcctgctgaaaatgactg

SEQ ID NO: 120 - DNA, 20 bp, synthetic construct
  source 1..20: mol_type = other DNA
gctgtatcgtcaaggcactc

SEQ ID NO: 121 - DNA, 27 bp, synthetic construct
  source 1..27: mol_type = other DNA
tgacactcattctctcaactctcatct

SEQ ID NO: 122 - DNA, 15 bp, synthetic construct
  source 1..15: mol_type = other DNA
  modified_base 8: mod_base = OTHER; note = LNA (locked nucleic acid)
acgccacaagctcca

SEQ ID NO: 123 - DNA, 17 bp, synthetic construct
  source 1..17: mol_type = other DNA
  modified_base 8: mod_base = OTHER; note = LNA (locked nucleic acid)
tacgccaacagctccaa

SEQ ID NO: 124 - DNA, 13 bp, synthetic construct
  source 1..13: mol_type = other DNA
  modified_base 7: mod_base = OTHER; note = LNA (locked nucleic acid)
cgccactagctcc

SEQ ID NO: 125 - DNA, 19 bp, synthetic construct
  source 1..19: mol_type = other DNA
  modified_base 9: mod_base = OTHER; note = LNA (locked nucleic acid)
ctacgccagcagctccaac

SEQ ID NO: 126 - DNA, 19 bp, synthetic construct
  source 1..19: mol_type = other DNA
  modified_base 9: mod_base = OTHER; note = LNA (locked nucleic acid)
ctacgccatcagctccaac

SEQ ID NO: 127 - DNA, 16 bp, synthetic construct
  source 1..16: mol_type = other DNA
  modified_base 8: mod_base = OTHER; note = LNA (locked nucleic acid)
acgccacgagctccaa

SEQ ID NO: 128 - DNA, 15 bp, synthetic construct
  source 1..15: mol_type = other DNA
  modified_base 1: mod_base = OTHER; note = 52MOEr (5' 2'-O-Methoxyethyl)
  modified_base 2: mod_base = OTHER; note = i2MOEr (int 2'-O-Methoxyethyl)
  modified_base 3: mod_base = OTHER; note = i2MOEr (int 2'-O-Methoxyethyl)
  modified_base 4..5: mod_base = OTHER; note = LNA (locked nucleic acid)
  modified_base 8: mod_base = OTHER; note = LNA (locked nucleic acid)
  misc_feature 15: note = 3SpC3 (3' blocker)
acgccaccagctcca

SEQ ID NO: 129 - DNA, 14 bp, synthetic construct
  source 1..14: mol_type = other DNA
  modified_base 8: mod_base = OTHER; note = LNA (locked nucleic acid)
tacgccagcagctc

SEQ ID NO: 130 - DNA, 15 bp, synthetic construct
  source 1..15: mol_type = other DNA
  modified_base 9: mod_base = OTHER; note = LNA (locked nucleic acid)
tacgccacgagctcc

SEQ ID NO: 131 - DNA, 14 bp, synthetic construct
  source 1..14: mol_type = other DNA
  modified_base 6: mod_base = OTHER; note = LNA (locked nucleic acid)
cgccatcagctcca

SEQ ID NO: 132 - DNA, 20 bp, synthetic construct
  source 1..20: mol_type = other DNA
  modified_base 9: mod_base = OTHER; note = LNA (locked nucleic acid)
ctacgccaacagctccaact

SEQ ID NO: 133 - DNA, 18 bp, synthetic construct
  source 1..18: mol_type = other DNA
  modified_base 11: mod_base = OTHER; note = LNA (locked nucleic acid)
cctacgccacaagctcca

SEQ ID NO: 134 - DNA, 17 bp, synthetic construct
  source 1..17: mol_type = other DNA
  modified_base 7: mod_base = OTHER; note = LNA (locked nucleic acid)
ctacgcaaccagctcca

SEQ ID NO: 135 - DNA, 16 bp, synthetic construct
  source 1..16: mol_type = other DNA
  modified_base 7: mod_base = OTHER; note = LNA (locked nucleic acid)
cctacgtcaccagctc

SEQ ID NO: 136 - DNA, 20 bp, synthetic construct
  source 1..20: mol_type = other DNA
aggcctgctgaaaatgactg

SEQ ID NO: 137 - DNA, 20 bp, synthetic construct
  source 1..20: mol_type = other DNA
gctgtatcgtcaaggcactc

SEQ ID NO: 138 - DNA, 22 bp, synthetic construct
  source 1..22: mol_type = other DNA
cccatgacacaatccagcttta

SEQ ID NO: 139 - DNA, 22 bp, synthetic construct
  source 1..22: mol_type = other DNA
aagcatcatggcagtagttctc

SEQ ID NO: 140 - DNA, 27 bp, synthetic construct
  source 1..27: mol_type = other DNA
  modified_base 1: mod_base = OTHER; note = 5' Quasar 705
  modified_base 27: mod_base = OTHER; note = 3' BHQ-2
tgacactcattctctcaactctcatct

SEQ ID NO: 141 - DNA, 15 bp, synthetic construct
  source 1..15: mol_type = other DNA
  modified_base 1: mod_base = OTHER; note = 5' 6-FAM (fluorescein)
  modified_base 8: mod_base = OTHER; note = LNA (locked nucleic acid)
  modified_base 15: mod_base = OTHER; note = 3IABkFQ (3' Iowa Black FQ)
acgccacaagctcca

SEQ ID NO: 142 - DNA, 17 bp, synthetic construct
  source 1..17: mol_type = other DNA
  modified_base 1: mod_base = OTHER; note = 5' TEX 615
  modified_base 8: mod_base = OTHER; note = LNA (locked nucleic acid)
  modified_base 17: mod_base = OTHER; note = 3IAbRQSp (3' Iowa Black RQ)
tacgccaacagctccaa

SEQ ID NO: 143 - DNA, 13 bp, synthetic construct
  source 1..13: mol_type = other DNA
  modified_base 1: mod_base = OTHER; note = 5' HEX
  modified_base 7: mod_base = OTHER; note = LNA (locked nucleic acid)
  modified_base 13: mod_base = OTHER; note = 3IABkFQ (3' Iowa Black FQ)
cgccactagctcc

SEQ ID NO: 144 - DNA, 14 bp, synthetic construct
  source 1..14: mol_type = other DNA
  modified_base 1: mod_base = OTHER; note = 5' 6-FAM
  modified_base 8: mod_base = OTHER; note = LNA (locked nucleic acid)
  modified_base 14: mod_base = OTHER; note = 3IABkFQ (3' Iowa Black FQ)
tacgccagcagctc

SEQ ID NO: 145 - DNA, 15 bp, synthetic construct
  source 1..15: mol_type = other DNA
  modified_base 1: mod_base = OTHER; note = 5TEX615
  modified_base 9: mod_base = OTHER; note = LNA (locked nucleic acid)
  modified_base 15: mod_base = OTHER; note = 3IAbRQSp (3' Iowa Black RQ)
tacgccacgagctcc

SEQ ID NO: 146 - DNA, 14 bp, synthetic construct
  source 1..14: mol_type = other DNA
  modified_base 1: mod_base = OTHER; note = 5' HEX
  modified_base 6: mod_base = OTHER; note = LNA (locked nucleic acid)
  modified_base 14: mod_base = OTHER; note = 3IABkFQ (3' Iowa Black FQ)
cgccatcagctcca

SEQ ID NO: 147 - DNA, 17 bp, synthetic construct
  source 1..17: mol_type = other DNA
  modified_base 1: mod_base = OTHER; note = 5' HEX
  modified_base 7: mod_base = OTHER; note = LNA (locked nucleic acid)
  modified_base 17: mod_base = OTHER; note = 3IABkFQ (3' Iowa Black FQ)
ctacgcaaccagctcca

SEQ ID NO: 148 - DNA, 16 bp, synthetic construct
  source 1..16: mol_type = other DNA
  modified_base 1: mod_base = OTHER; note = 5' 6-FAM
  modified_base 7: mod_base = OTHER; note = LNA (locked nucleic acid)
  modified_base 16: mod_base = OTHER; note = 3IABkFQ (3' Iowa Black FQ)
cctacgtcaccagctc

SEQ ID NO: 149 - DNA, 17 bp, synthetic construct
  source 1..17: mol_type = other DNA
  modified_base 1..2: mod_base = OTHER; note = 2'-amino-2'-deoxycytidine (2-Am)
  modified_base 7..8: mod_base = OTHER; note = LNA (locked nucleic acid)
  modified_base 11: mod_base = OTHER; note = LNA (locked nucleic acid)
  misc_feature 17: note = 3SpC3 (3' C3 spacer)
cctacgccaccagctcc

SEQ ID NO: 150 - DNA, 16 bp, synthetic construct
  source 1..16: mol_type = other DNA
  modified_base 1..3: mod_base = OTHER; note = 2'-fluorinated nucleosides
  modified_base 7..8: mod_base = OTHER; note = LNA (locked nucleic acid)
  modified_base 11: mod_base = OTHER; note = LNA (locked nucleic acid)
  misc_feature 16: note = 3SpC3 (3' C3 spacer)
  modified_base 2..4: mod_base = OTHER; note = Phosphorothioate Bond
cctacgccaccagtcc

SEQ ID NO: 151 - DNA, 20 bp, synthetic construct
  source 1..20: mol_type = other DNA
  modified_base 12..13: mod_base = OTHER; note = LNA (locked nucleic acid)
  modified_base 16: mod_base = OTHER; note = LNA (locked nucleic acid)
  misc_feature 20: note = 3SpC3 (3' C3 spacer)
gcgaagctacgccaccagct

SEQ ID NO: 152 - DNA, 19 bp, synthetic construct
  source 1..19: mol_type = other DNA
tggtagttggagctggtga

SEQ ID NO: 153 - DNA, 21 bp, synthetic construct
  source 1..21: mol_type = other DNA
acttgtggtagttggagctga

SEQ ID NO: 154 - DNA, 28 bp, synthetic construct
  source 1..28: mol_type = other DNA
gaatataaacttgtggtagttggagctt

SEQ ID NO: 155 - DNA, 14 bp, synthetic construct
  source 1..14: mol_type = other DNA
  modified_base 13..14: mod_base = OTHER; note = Locked Nucleic Acid (LNA)
  misc_feature 1: note = 3' Spacer (3SpC3)
  misc_feature 14: note = 3' Spacer (3SpC3)
ttgcctacgccacc

SEQ ID NO: 156 - DNA, 18 bp, synthetic construct
  source 1..18: mol_type = other DNA
  modified_base 14..15: mod_base = OTHER; note = Locked Nucleic Acid (LNA)
  modified_base 18: mod_base = OTHER; note = Locked Nucleic Acid (LNA)
ggtagttggagctggtgg